SEQUENCE LISTING (230 sequences)

SEQ ID NO: 1 - DNA, 32 bp, synthetic construct
  source 1..32: mol_type = other DNA
taaattgttagctgataaattaataaagggtg

SEQ ID NO: 2 - DNA, 32 bp, synthetic construct
  source 1..32: mol_type = other DNA
ctggctgacgcgaaacaaagtacagggtaaaa

SEQ ID NO: 3 - DNA, 29 bp, synthetic construct
  source 1..29: mol_type = other DNA
agaaaggccggagacagtcaaatcaccat

SEQ ID NO: 4 - DNA, 32 bp, synthetic construct
  source 1..32: mol_type = other DNA
atatttaaataaagtaccgacaaattacgagc

SEQ ID NO: 5 - DNA, 32 bp, synthetic construct
  source 1..32: mol_type = other DNA
gattcatctaacaaagctgctcattcttgaca

SEQ ID NO: 6 - DNA, 32 bp, synthetic construct
  source 1..32: mol_type = other DNA
tatccggtattctaagtcagatatagaaggct

SEQ ID NO: 7 - DNA, 32 bp, synthetic construct
  source 1..32: mol_type = other DNA
gcttttgcgcggagtgagaatagagacgttag

SEQ ID NO: 8 - DNA, 41 bp, synthetic construct
  source 1..41: mol_type = other DNA
gggagctaaacagatcaaaatcaccagtgccttgagtagga

SEQ ID NO: 9 - DNA, 32 bp, synthetic construct
  source 1..32: mol_type = other DNA
ctgtcgtgaaaacgacggccagtgatgtgctg

SEQ ID NO: 10 - DNA, 32 bp, synthetic construct
  source 1..32: mol_type = other DNA
agtccactttcaccagtgagacggaattgcgt

SEQ ID NO: 11 - DNA, 32 bp, synthetic construct
  source 1..32: mol_type = other DNA
cattttcagtacaaactacaacgctccaaaag

SEQ ID NO: 12 - DNA, 32 bp, synthetic construct
  source 1..32: mol_type = other DNA
attcatcaacagtaccttttacatatttcaat

SEQ ID NO: 13 - DNA, 19 bp, synthetic construct
  source 1..19: mol_type = other DNA
aggtctttaccctgactat

SEQ ID NO: 14 - DNA, 32 bp, synthetic construct
  source 1..32: mol_type = other DNA
agaaccggactcatctttgacccccaacctaa

SEQ ID NO: 15 - DNA, 32 bp, synthetic construct
  source 1..32: mol_type = other DNA
tatagtcaaacgagaatgaccatagagggggt

SEQ ID NO: 16 - DNA, 32 bp, synthetic construct
  source 1..32: mol_type = other DNA
tacgtaatacaatgacaacaaccatctccaaa

SEQ ID NO: 17 - DNA, 32 bp, synthetic construct
  source 1..32: mol_type = other DNA
atttttgttaaatcagttaaaattcgcattaa

SEQ ID NO: 18 - DNA, 32 bp, synthetic construct
  source 1..32: mol_type = other DNA
ggtgccgtacgagcacgtataacgccagaatc

SEQ ID NO: 19 - DNA, 32 bp, synthetic construct
  source 1..32: mol_type = other DNA
taaatgaaataggtgtatcaccgtaaggatta

SEQ ID NO: 20 - DNA, 30 bp, synthetic construct
  source 1..30: mol_type = other DNA
attaatgaatcgggtaccgtaacacagctt

SEQ ID NO: 21 - DNA, 32 bp, synthetic construct
  source 1..32: mol_type = other DNA
aactaacggccctgacgagaaacacgcatagg

SEQ ID NO: 22 - DNA, 32 bp, synthetic construct
  source 1..32: mol_type = other DNA
tatctaaaactcgtattaaatcctttggatta

SEQ ID NO: 23 - DNA, 34 bp, synthetic construct
  source 1..34: mol_type = other DNA
gctttttttcccagtcacgacgttgtccagctgc

SEQ ID NO: 24 - DNA, 22 bp, synthetic construct
  source 1..22: mol_type = other DNA
ggtgccggaaatatccgctcac

SEQ ID NO: 25 - DNA, 32 bp, synthetic construct
  source 1..32: mol_type = other DNA
aagggttatacaaacaattcgacaatatcttt

SEQ ID NO: 26 - DNA, 32 bp, synthetic construct
  source 1..32: mol_type = other DNA
cgcctgcacagagatagaaccctttgcaacag

SEQ ID NO: 27 - DNA, 32 bp, synthetic construct
  source 1..32: mol_type = other DNA
cctggccctggggtgcctaatgagagctcgaa

SEQ ID NO: 28 - DNA, 32 bp, synthetic construct
  source 1..32: mol_type = other DNA
gagcttgagcccgagatagggttgcttcaccg

SEQ ID NO: 29 - DNA, 32 bp, synthetic construct
  source 1..32: mol_type = other DNA
tctttccaaaggaacaactaaagggaggcttg

SEQ ID NO: 30 - DNA, 32 bp, synthetic construct
  source 1..32: mol_type = other DNA
tcgttagaccaaatcaagtttttttccaacgt

SEQ ID NO: 31 - DNA, 32 bp, synthetic construct
  source 1..32: mol_type = other DNA
cagttgattcccaattgtttcattccatataa

SEQ ID NO: 32 - DNA, 32 bp, synthetic construct
  source 1..32: mol_type = other DNA
ggattagctcctcattaaagccagcaccacca

SEQ ID NO: 33 - DNA, 32 bp, synthetic construct
  source 1..32: mol_type = other DNA
gtctggaactgcgaacgagtagattgaaaagg

SEQ ID NO: 34 - DNA, 32 bp, synthetic construct
  source 1..32: mol_type = other DNA
gaaaaacgtgtttttataatcagttactatgg

SEQ ID NO: 35 - DNA, 32 bp, synthetic construct
  source 1..32: mol_type = other DNA
cccgacttaggaatcattaccgcgtcaacaat

SEQ ID NO: 36 - DNA, 34 bp, synthetic construct
  source 1..34: mol_type = other DNA
aaatcgcgtaaatcgtcgctattaattaagcatt

SEQ ID NO: 37 - DNA, 42 bp, synthetic construct
  source 1..42: mol_type = other DNA
attacgcagtatgttagcaaaaccattaccattagcaaggcc

SEQ ID NO: 38 - DNA, 32 bp, synthetic construct
  source 1..32: mol_type = other DNA
aataacataaactggcatgattaagactcctt

SEQ ID NO: 39 - DNA, 32 bp, synthetic construct
  source 1..32: mol_type = other DNA
tcaccgtcagcgtcagactgtagccagaaccg

SEQ ID NO: 40 - DNA, 32 bp, synthetic construct
  source 1..32: mol_type = other DNA
acgttattcagttgaaaggaattgattaacac

SEQ ID NO: 41 - DNA, 32 bp, synthetic construct
  source 1..32: mol_type = other DNA
actggtaaaacagttaatgcccccaccaccct

SEQ ID NO: 42 - DNA, 32 bp, synthetic construct
  source 1..32: mol_type = other DNA
tttatcaaaatcatagaagagtcaatagtgaa

SEQ ID NO: 43 - DNA, 32 bp, synthetic construct
  source 1..32: mol_type = other DNA
cctcagaagaaagtattaagaggctctgaatt

SEQ ID NO: 44 - DNA, 32 bp, synthetic construct
  source 1..32: mol_type = other DNA
aattcataaggtaaatattgacggtttcggtc

SEQ ID NO: 45 - DNA, 32 bp, synthetic construct
  source 1..32: mol_type = other DNA
atactgcgattcaactaatgcagaggtagaaa

SEQ ID NO: 46 - DNA, 32 bp, synthetic construct
  source 1..32: mol_type = other DNA
tagccagcgaaaagccccaaaaacatttttga

SEQ ID NO: 47 - DNA, 32 bp, synthetic construct
  source 1..32: mol_type = other DNA
aatacatttgaggattagagccgtcaatagat

SEQ ID NO: 48 - DNA, 32 bp, synthetic construct
  source 1..32: mol_type = other DNA
tagctgttcataaagtgtaaagcctgagagag

SEQ ID NO: 49 - DNA, 32 bp, synthetic construct
  source 1..32: mol_type = other DNA
gatgatactcagagccgccaccctgcgttttc

SEQ ID NO: 50 - DNA, 32 bp, synthetic construct
  source 1..32: mol_type = other DNA
tgcgctcagcaggtcgactctagatcttcgct

SEQ ID NO: 51 - DNA, 32 bp, synthetic construct
  source 1..32: mol_type = other DNA
aagagtaatcagtgaataaggcttgaacaaca

SEQ ID NO: 52 - DNA, 34 bp, synthetic construct
  source 1..34: mol_type = other DNA
tcatataacctgtttagctatatttttaatactt

SEQ ID NO: 53 - DNA, 32 bp, synthetic construct
  source 1..32: mol_type = other DNA
tgaccctgcatttggggcgcgagcttagtttg

SEQ ID NO: 54 - DNA, 30 bp, synthetic construct
  source 1..30: mol_type = other DNA
ttgcgggagaagcaggaataccacgaatcg

SEQ ID NO: 55 - DNA, 30 bp, synthetic construct
  source 1..30: mol_type = other DNA
aattgctgaatataagacttcaaaaggtca

SEQ ID NO: 56 - DNA, 32 bp, synthetic construct
  source 1..32: mol_type = other DNA
tgaaacatccgccaccctcagaacacagccct

SEQ ID NO: 57 - DNA, 34 bp, synthetic construct
  source 1..34: mol_type = other DNA
ggattagagagtacctttaattgctctagagctt

SEQ ID NO: 58 - DNA, 30 bp, synthetic construct
  source 1..30: mol_type = other DNA
ggtaacgccagggcgaggtgaattaggcaa

SEQ ID NO: 59 - DNA, 32 bp, synthetic construct
  source 1..32: mol_type = other DNA
agtaacaaatgtcaatcatatgtaattgcctg

SEQ ID NO: 60 - DNA, 34 bp, synthetic construct
  source 1..34: mol_type = other DNA
aagaaacaaacggcggattgaccgtattaagttg

SEQ ID NO: 61 - DNA, 30 bp, synthetic construct
  source 1..30: mol_type = other DNA
ctaacgaataagagcaactgaacctcaaat

SEQ ID NO: 62 - DNA, 32 bp, synthetic construct
  source 1..32: mol_type = other DNA
tacctgagtcaatatatgtgagtgagattaag

SEQ ID NO: 63 - DNA, 32 bp, synthetic construct
  source 1..32: mol_type = other DNA
acgctgaggtctgagagactacctttttaacc

SEQ ID NO: 64 - DNA, 32 bp, synthetic construct
  source 1..32: mol_type = other DNA
atgtagaaaagtaccgcactcatcgttgctat

SEQ ID NO: 65 - DNA, 32 bp, synthetic construct
  source 1..32: mol_type = other DNA
acgctggtacatacgagccggaagtcctgtgt

SEQ ID NO: 66 - DNA, 32 bp, synthetic construct
  source 1..32: mol_type = other DNA
ttgcagcaataaatcaaaagaatacggggaaa

SEQ ID NO: 67 - DNA, 32 bp, synthetic construct
  source 1..32: mol_type = other DNA
gggagcccgccgctacagggcgcggaggccac

SEQ ID NO: 68 - DNA, 32 bp, synthetic construct
  source 1..32: mol_type = other DNA
atattttgctcattttttaaccaactcaggaa

SEQ ID NO: 69 - DNA, 32 bp, synthetic construct
  source 1..32: mol_type = other DNA
acgcgagaaaacttttaatcgcaagacaaaga

SEQ ID NO: 70 - DNA, 32 bp, synthetic construct
  source 1..32: mol_type = other DNA
caaataaaggggttttgctcagtaataagtat

SEQ ID NO: 71 - DNA, 32 bp, synthetic construct
  source 1..32: mol_type = other DNA
atttttgcggatggctcttttgataagaggtc

SEQ ID NO: 72 - DNA, 32 bp, synthetic construct
  source 1..32: mol_type = other DNA
tttttcttattaaagaacgtggacggggtcga

SEQ ID NO: 73 - DNA, 30 bp, synthetic construct
  source 1..30: mol_type = other DNA
cataattttaggcagagttttcccttagaa

SEQ ID NO: 74 - DNA, 32 bp, synthetic construct
  source 1..32: mol_type = other DNA
ctgaacaaaaataagaaacgatttgcgaacct

SEQ ID NO: 75 - DNA, 32 bp, synthetic construct
  source 1..32: mol_type = other DNA
atcattttctcaatcaatatctggagccagca

SEQ ID NO: 76 - DNA, 32 bp, synthetic construct
  source 1..32: mol_type = other DNA
gagcctttaccgatagttgcgccggccactac

SEQ ID NO: 77 - DNA, 29 bp, synthetic construct
  source 1..29: mol_type = other DNA
agcccggattttctgtatgggattttgct

SEQ ID NO: 78 - DNA, 35 bp, synthetic construct
  source 1..35: mol_type = other DNA
gcgcagacggtcaatcataagggaacctgctccat

SEQ ID NO: 79 - DNA, 32 bp, synthetic construct
  source 1..32: mol_type = other DNA
ccagtagccgtagaaaatacatacggaatacc

SEQ ID NO: 80 - DNA, 30 bp, synthetic construct
  source 1..30: mol_type = other DNA
attcaaatcttttcatagaggccgattaaa

SEQ ID NO: 81 - DNA, 32 bp, synthetic construct
  source 1..32: mol_type = other DNA
caaatcaaaattttaaaagtttgatgatggca

SEQ ID NO: 82 - DNA, 32 bp, synthetic construct
  source 1..32: mol_type = other DNA
tatcagagaaaataaacagccataccttaaat

SEQ ID NO: 83 - DNA, 32 bp, synthetic construct
  source 1..32: mol_type = other DNA
aaaaaggcctgtagcattccacagcgccaccc

SEQ ID NO: 84 - DNA, 34 bp, synthetic construct
  source 1..34: mol_type = other DNA
atcaaaccgcggaacaaagaaaccaccagcaacg

SEQ ID NO: 85 - DNA, 34 bp, synthetic construct
  source 1..34: mol_type = other DNA
tccttgaaaggttgggttatataactataggaat

SEQ ID NO: 86 - DNA, 32 bp, synthetic construct
  source 1..32: mol_type = other DNA
tacttctgtttcaggtttaacgtcaacaaaca

SEQ ID NO: 87 - DNA, 32 bp, synthetic construct
  source 1..32: mol_type = other DNA
ataataatgcgtaacgatctaaagaccgccac

SEQ ID NO: 88 - DNA, 32 bp, synthetic construct
  source 1..32: mol_type = other DNA
atcggcataaattattcattaaagtttatttt

SEQ ID NO: 89 - DNA, 32 bp, synthetic construct
  source 1..32: mol_type = other DNA
tcaagaaaaattaccttttttaatggaaacag

SEQ ID NO: 90 - DNA, 34 bp, synthetic construct
  source 1..34: mol_type = other DNA
gcaaatcctcaaatatattttagttaatttcatc

SEQ ID NO: 91 - DNA, 45 bp, synthetic construct
  source 1..45: mol_type = other DNA
ggaaacgtcaccaatgaaaccgccagcattgacaggattcacaaa

SEQ ID NO: 92 - DNA, 32 bp, synthetic construct
  source 1..32: mol_type = other DNA
ttaacaatttcatttgacaaaattaattacat

SEQ ID NO: 93 - DNA, 42 bp, synthetic construct
  source 1..42: mol_type = other DNA
ctgtttgatggtggttccgaaagggaagaaagcgaaaggagc

SEQ ID NO: 94 - DNA, 32 bp, synthetic construct
  source 1..32: mol_type = other DNA
gccggcgacgcgtaaccaccacacgcaaatta

SEQ ID NO: 95 - DNA, 32 bp, synthetic construct
  source 1..32: mol_type = other DNA
gtatcggctaggaacgccatcaaacaaatatt

SEQ ID NO: 96 - DNA, 32 bp, synthetic construct
  source 1..32: mol_type = other DNA
gaaagacagcatcggaacgaggccggaacgag

SEQ ID NO: 97 - DNA, 32 bp, synthetic construct
  source 1..32: mol_type = other DNA
cgagtaaatattaccgccagccatctgacctg

SEQ ID NO: 98 - DNA, 34 bp, synthetic construct
  source 1..34: mol_type = other DNA
gggattttcgtctgaaatggattatttacgcgac

SEQ ID NO: 99 - DNA, 32 bp, synthetic construct
  source 1..32: mol_type = other DNA
atgggcgcagctggcgaaagggggccaagctt

SEQ ID NO: 100 - DNA, 32 bp, synthetic construct
  source 1..32: mol_type = other DNA
atacagtaatataatcctgattgtttgcccga

SEQ ID NO: 101 - DNA, 32 bp, synthetic construct
  source 1..32: mol_type = other DNA
ctggccaaacagtgccacgctgagtcagttgg

SEQ ID NO: 102 - DNA, 32 bp, synthetic construct
  source 1..32: mol_type = other DNA
acatccaaaattaagcaataaagctgtaggta

SEQ ID NO: 103 - DNA, 32 bp, synthetic construct
  source 1..32: mol_type = other DNA
tacataaacaaaagaagatgatgaagatgaat

SEQ ID NO: 104 - DNA, 32 bp, synthetic construct
  source 1..32: mol_type = other DNA
ttgcctttaccgacttgagccattataaaaga

SEQ ID NO: 105 - DNA, 32 bp, synthetic construct
  source 1..32: mol_type = other DNA
agagccagtaatcagtagcgacaggagccgcc

SEQ ID NO: 106 - DNA, 32 bp, synthetic construct
  source 1..32: mol_type = other DNA
caaagggctttgcgtattgggcgccgggaaac

SEQ ID NO: 107 - DNA, 32 bp, synthetic construct
  source 1..32: mol_type = other DNA
actccaactatcgcgttttaattccatcaaaa

SEQ ID NO: 108 - DNA, 32 bp, synthetic construct
  source 1..32: mol_type = other DNA
ggcaaagaattagcaataaatcatacaggcaa

SEQ ID NO: 109 - DNA, 29 bp, synthetic construct
  source 1..29: mol_type = other DNA
caatatgatattcaaccgttcaaacgtta

SEQ ID NO: 110 - DNA, 32 bp, synthetic construct
  source 1..32: mol_type = other DNA
ccaccctccagtaagcgtcatacatattattc

SEQ ID NO: 111 - DNA, 32 bp, synthetic construct
  source 1..32: mol_type = other DNA
gtcacaataagaaaagtaagcagaagcgctaa

SEQ ID NO: 112 - DNA, 32 bp, synthetic construct
  source 1..32: mol_type = other DNA
ccgcgaccgaactgaccaactttgtttaattt

SEQ ID NO: 113 - DNA, 32 bp, synthetic construct
  source 1..32: mol_type = other DNA
caaagttagacaccacggaataaggtgaatta

SEQ ID NO: 114 - DNA, 32 bp, synthetic construct
  source 1..32: mol_type = other DNA
catagttatttttcacgttgaaaatcgcccac

SEQ ID NO: 115 - DNA, 19 bp, synthetic construct
  source 1..19: mol_type = other DNA
cgaaccagaccggaagcaa

SEQ ID NO: 116 - DNA, 32 bp, synthetic construct
  source 1..32: mol_type = other DNA
cccaatccagtcagagggtaattgtagccgaa

SEQ ID NO: 117 - DNA, 31 bp, synthetic construct
  source 1..31: mol_type = other DNA
gttgggaattgggcttgagatggaaagagga

SEQ ID NO: 118 - DNA, 32 bp, synthetic construct
  source 1..32: mol_type = other DNA
tggcatcaaaatcggttgtaccaatcatatat

SEQ ID NO: 119 - DNA, 45 bp, synthetic construct
  source 1..45: mol_type = other DNA
tttgcacgtaaaacagaaataaagaaattgcgtagataataatgg

SEQ ID NO: 120 - DNA, 32 bp, synthetic construct
  source 1..32: mol_type = other DNA
ttctttgatgtagcggtcacgctgacgtggcg

SEQ ID NO: 121 - DNA, 32 bp, synthetic construct
  source 1..32: mol_type = other DNA
tagccctaaaacatcgccattaaaaatgctat

SEQ ID NO: 122 - DNA, 32 bp, synthetic construct
  source 1..32: mol_type = other DNA
ttcatgaggatatattcggtcgctaattgcga

SEQ ID NO: 123 - DNA, 32 bp, synthetic construct
  source 1..32: mol_type = other DNA
acatgtaatactagaaaaagcctgataaggcg

SEQ ID NO: 124 - DNA, 32 bp, synthetic construct
  source 1..32: mol_type = other DNA
agagtctgaaaatttttagaacccaaacatta

SEQ ID NO: 125 - DNA, 32 bp, synthetic construct
  source 1..32: mol_type = other DNA
gtctatcactacgtgaaccatcacatcagagc

SEQ ID NO: 126 - DNA, 32 bp, synthetic construct
  source 1..32: mol_type = other DNA
tttaaatgcaaaggctatcaggtcccccggtt

SEQ ID NO: 127 - DNA, 32 bp, synthetic construct
  source 1..32: mol_type = other DNA
tttgcaccctaatttgccagttacagataacc

SEQ ID NO: 128 - DNA, 32 bp, synthetic construct
  source 1..32: mol_type = other DNA
attttatcgaacgggtattaaaccaccaatca

SEQ ID NO: 129 - DNA, 19 bp, synthetic construct
  source 1..19: mol_type = other DNA
caacgctcaactccagacg

SEQ ID NO: 130 - DNA, 32 bp, synthetic construct
  source 1..32: mol_type = other DNA
ctgagaagctcatggaaatacctagggacatt

SEQ ID NO: 131 - DNA, 25 bp, synthetic construct
  source 1..25: mol_type = other DNA
acttgaacctaccatatcaaaatta

SEQ ID NO: 132 - DNA, 32 bp, synthetic construct
  source 1..32: mol_type = other DNA
aagcccaataacagtgcccgtatataagtttt

SEQ ID NO: 133 - DNA, 32 bp, synthetic construct
  source 1..32: mol_type = other DNA
ataatcggccagtaataagagaatcaacgcca

SEQ ID NO: 134 - DNA, 32 bp, synthetic construct
  source 1..32: mol_type = other DNA
ttcatcgtgcgggaggttttgaagttatttat

SEQ ID NO: 135 - DNA, 32 bp, synthetic construct
  source 1..32: mol_type = other DNA
taccgttcagagccaccaccctcaaatcaagt

SEQ ID NO: 136 - DNA, 32 bp, synthetic construct
  source 1..32: mol_type = other DNA
aattccacacattgccccagcaggcgaaaatc

SEQ ID NO: 137 - DNA, 32 bp, synthetic construct
  source 1..32: mol_type = other DNA
tggcacaggtaatatccagaacaaagagtctg

SEQ ID NO: 138 - DNA, 32 bp, synthetic construct
  source 1..32: mol_type = other DNA
gagatctacaatgcctgagtaatgctcagagc

SEQ ID NO: 139 - DNA, 32 bp, synthetic construct
  source 1..32: mol_type = other DNA
agaaaggaatcggcaaaatcccttagcggtcc

SEQ ID NO: 140 - DNA, 32 bp, synthetic construct
  source 1..32: mol_type = other DNA
acgacaatcctgaacaagaaaaattttttatt

SEQ ID NO: 141 - DNA, 30 bp, synthetic construct
  source 1..30: mol_type = other DNA
ttcgagctgtctttcctataccaagttaca

SEQ ID NO: 142 - DNA, 30 bp, synthetic construct
  source 1..30: mol_type = other DNA
cgcaaatggtcaaaatattcattgaggaag

SEQ ID NO: 143 - DNA, 40 bp, synthetic construct
  source 1..40: mol_type = other DNA
cgtcgagagggttgatccaggcggataagtgcacgattgg

SEQ ID NO: 144 - DNA, 34 bp, synthetic construct
  source 1..34: mol_type = other DNA
cccgaaatgctgtagctcaacatgttatacattt

SEQ ID NO: 145 - DNA, 32 bp, synthetic construct
  source 1..32: mol_type = other DNA
gttacttaggtagcaacggctacataaaggcc

SEQ ID NO: 146 - DNA, 24 bp, synthetic construct
  source 1..24: mol_type = other DNA
ccttgataggttgaggcaggtcag

SEQ ID NO: 147 - DNA, 32 bp, synthetic construct
  source 1..32: mol_type = other DNA
accgttgtactatcggccttgctgacaatatt

SEQ ID NO: 148 - DNA, 32 bp, synthetic construct
  source 1..32: mol_type = other DNA
tccatcacccgccgcgcttaatgcccgattta

SEQ ID NO: 149 - DNA, 32 bp, synthetic construct
  source 1..32: mol_type = other DNA
ctcaagagactcaggaggtttagtttttgtcg

SEQ ID NO: 150 - DNA, 35 bp, synthetic construct
  source 1..35: mol_type = other DNA
caactttaactggctcattataccagacgacgata

SEQ ID NO: 151 - DNA, 20 bp, synthetic construct
  source 1..20: mol_type = other DNA
aatagatttagaagtattag

SEQ ID NO: 152 - DNA, 32 bp, synthetic construct
  source 1..32: mol_type = other DNA
gggcgctagggcgctggcaagttagtaataac

SEQ ID NO: 153 - DNA, 35 bp, synthetic construct
  source 1..35: mol_type = other DNA
ccgtgtgataatttagtatcatatgcggaatcgcc

SEQ ID NO: 154 - DNA, 32 bp, synthetic construct
  source 1..32: mol_type = other DNA
gactggattttaaacagttcagaagaagcaaa

SEQ ID NO: 155 - DNA, 19 bp, synthetic construct
  source 1..19: mol_type = other DNA
gcggattggagcttcaaag

SEQ ID NO: 156 - DNA, 32 bp, synthetic construct
  source 1..32: mol_type = other DNA
accagaacaatggaaagcgcagtctgagactc

SEQ ID NO: 157 - DNA, 32 bp, synthetic construct
  source 1..32: mol_type = other DNA
acgtcaaaagacgggagaattaacgaaacgca

SEQ ID NO: 158 - DNA, 35 bp, synthetic construct
  source 1..35: mol_type = other DNA
acccatccaacgcgcggggagaggcgggaaaaacc

SEQ ID NO: 159 - DNA, 42 bp, synthetic construct
  source 1..42: mol_type = other DNA
aaacaactttcaacagtttcagggatcgtcaccctcagcagc

SEQ ID NO: 160 - DNA, 19 bp, synthetic construct
  source 1..19: mol_type = other DNA
aaaaccaaaatagcgagag

SEQ ID NO: 161 - DNA, 32 bp, synthetic construct
  source 1..32: mol_type = other DNA
gcataaccgaagtttccattaaacacggagat

SEQ ID NO: 162 - DNA, 32 bp, synthetic construct
  source 1..32: mol_type = other DNA
caagattagagaacaagcaagccgaatatccc

SEQ ID NO: 163 - DNA, 33 bp, synthetic construct
  source 1..33: mol_type = other DNA
gcttttgcataaccctcgtttaccagtcaggac

SEQ ID NO: 164 - DNA, 32 bp, synthetic construct
  source 1..32: mol_type = other DNA
tgtataagaataattcgcgtctggtgccagtt

SEQ ID NO: 165 - DNA, 32 bp, synthetic construct
  source 1..32: mol_type = other DNA
cagcgccaatagcaatagctatctttgagtta

SEQ ID NO: 166 - DNA, 32 bp, synthetic construct
  source 1..32: mol_type = other DNA
tttgaatgaccgaacgaaccaccataacaact

SEQ ID NO: 167 - DNA, 32 bp, synthetic construct
  source 1..32: mol_type = other DNA
aggagcacgcagaagataaaacagagaatacg

SEQ ID NO: 168 - DNA, 32 bp, synthetic construct
  source 1..32: mol_type = other DNA
attacgccatcgtaaccgtgcatcccttcctg

SEQ ID NO: 169 - DNA, 32 bp, synthetic construct
  source 1..32: mol_type = other DNA
cagggagtgaggctttgaggactagtcgaaat

SEQ ID NO: 170 - DNA, 32 bp, synthetic construct
  source 1..32: mol_type = other DNA
aatagtaagaattacgaggcatagataaaacg

SEQ ID NO: 171 - DNA, 34 bp, synthetic construct
  source 1..34: mol_type = other DNA
attacaacggtaatcgtaaaactagccccgtcgg

SEQ ID NO: 172 - DNA, 30 bp, synthetic construct
  source 1..30: mol_type = other DNA
attctccgtgggatacactaaaacatattc

SEQ ID NO: 173 - DNA, 30 bp, synthetic construct
  source 1..30: mol_type = other DNA
ttccaactgaatcttacaaggagcggaatt

SEQ ID NO: 174 - DNA, 32 bp, synthetic construct
  source 1..32: mol_type = other DNA
aaagcgtaaggtgaggcggtcagtaggaaggt

SEQ ID NO: 175 - DNA, 32 bp, synthetic construct
  source 1..32: mol_type = other DNA
cgctcaatagacaggaacggtacgtgctttcc

SEQ ID NO: 176 - DNA, 32 bp, synthetic construct
  source 1..32: mol_type = other DNA
aacggggtcggaaccagagccacccttattag

SEQ ID NO: 177 - DNA, 32 bp, synthetic construct
  source 1..32: mol_type = other DNA
tgattgccagtgttgttccagtttaccctaaa

SEQ ID NO: 178 - DNA, 32 bp, synthetic construct
  source 1..32: mol_type = other DNA
cgtttgccccgattgagggagggatggtttac

SEQ ID NO: 179 - DNA, 30 bp, synthetic construct
  source 1..30: mol_type = other DNA
ttctgagaataaacacctgtaaatgctgat

SEQ ID NO: 180 - DNA, 32 bp, synthetic construct
  source 1..32: mol_type = other DNA
atagccccaccggaaccgcctcccaggagtgt

SEQ ID NO: 181 - DNA, 32 bp, synthetic construct
  source 1..32: mol_type = other DNA
accattagttaaatatgcaactaaagtacggt

SEQ ID NO: 182 - DNA, 19 bp, synthetic construct
  source 1..19: mol_type = other DNA
gcgcctgtttacccaatag

SEQ ID NO: 183 - DNA, 35 bp, synthetic construct
  source 1..35: mol_type = other DNA
tagtctttaatgagtagaagaactcaaagcaatac

SEQ ID NO: 184 - DNA, 35 bp, synthetic construct
  source 1..35: mol_type = other DNA
agataagtaaacaacatgttcagctaatgcagaac

SEQ ID NO: 185 - DNA, 34 bp, synthetic construct
  source 1..34: mol_type = other DNA
atcatcatggattcgcctgattgctttgatatca

SEQ ID NO: 186 - DNA, 32 bp, synthetic construct
  source 1..32: mol_type = other DNA
gggtaccgtgagctaactcacattgcaacagc

SEQ ID NO: 187 - DNA, 32 bp, synthetic construct
  source 1..32: mol_type = other DNA
ttatgcgattttaagaatcattgtgaattacc

SEQ ID NO: 188 - DNA, 32 bp, synthetic construct
  source 1..32: mol_type = other DNA
gcaaatgaacgaccagtaataaaacattttga

SEQ ID NO: 189 - DNA, 32 bp, synthetic construct
  source 1..32: mol_type = other DNA
caagcaaaaacgcgaggcgttttatttgttta

SEQ ID NO: 190 - DNA, 35 bp, synthetic construct
  source 1..35: mol_type = other DNA
ttaaataacctaaatttaatggtttgaaataccga

SEQ ID NO: 191 - DNA, 32 bp, synthetic construct
  source 1..32: mol_type = other DNA
gaaggcaccagcgattataccaagccttcatc

SEQ ID NO: 192 - DNA, 22 bp, synthetic construct
  source 1..22: mol_type = other DNA
atcacttgcctgcgcgaactga

SEQ ID NO: 193 - DNA, 32 bp, synthetic construct
  source 1..32: mol_type = other DNA
tgaggggagcgatcggtgcgggccggatcccc

SEQ ID NO: 194 - DNA, 32 bp, synthetic construct
  source 1..32: mol_type = other DNA
aagattcagccggagagggtagctaggaagat

SEQ ID NO: 195 - DNA, 32 bp, synthetic construct
  source 1..32: mol_type = other DNA
aacgcaaaccagaaggaaaccgagtgaacacc

SEQ ID NO: 196 - DNA, 34 bp, synthetic construct
  source 1..34: mol_type = other DNA
ccagtcacaaaatctaaagcatcaccttggaaac

SEQ ID NO: 197 - DNA, 32 bp, synthetic construct
  source 1..32: mol_type = other DNA
cacaagaataccgaagccctttttcaatagaa

SEQ ID NO: 198 - DNA, 32 bp, synthetic construct
  source 1..32: mol_type = other DNA
tcagagcctgcctatttcggaacctggctttt

SEQ ID NO: 199 - DNA, 35 bp, synthetic construct
  source 1..35: mol_type = other DNA
ttaattgattatacaaattcttaccagtataaagc

SEQ ID NO: 200 - DNA, 32 bp, synthetic construct
  source 1..32: mol_type = other DNA
agattaagaatccccctcaaatgcagcgtcca

SEQ ID NO: 201 - DNA, 32 bp, synthetic construct
  source 1..32: mol_type = other DNA
gcatgcctctgcccgctttccagtcagggtgg

SEQ ID NO: 202 - DNA, 32 bp, synthetic construct
  source 1..32: mol_type = other DNA
aacgaaagtcttaaacagcttgataattgtat

SEQ ID NO: 203 - DNA, 32 bp, synthetic construct
  source 1..32: mol_type = other DNA
cggtttatctgagtttcgtcaccagggatagc

SEQ ID NO: 204 - DNA, 32 bp, synthetic construct
  source 1..32: mol_type = other DNA
gagccgccatcgatagcagcaccgcaaaatca

SEQ ID NO: 205 - DNA, 35 bp, synthetic construct
  source 1..35: mol_type = other DNA
acactatcaaaagaagttttgccaaatcaaaaatc

SEQ ID NO: 206 - DNA, 32 bp, synthetic construct
  source 1..32: mol_type = other DNA
ataaagctattctactaatagtagtagcatta

SEQ ID NO: 207 - DNA, 19 bp, synthetic construct
  source 1..19: mol_type = other DNA
tagcagcctttacagagag

SEQ ID NO: 208 - DNA, 32 bp, synthetic construct
  source 1..32: mol_type = other DNA
caaaagaaaacagggaagcgcattaatgaaaa

SEQ ID NO: 209 - DNA, 34 bp, synthetic construct
  source 1..34: mol_type = other DNA
gatttctttatttcaacgcaaggatagagcaaac

SEQ ID NO: 210 - DNA, 32 bp, synthetic construct
  source 1..32: mol_type = other DNA
tccggcttaacatagcgatagcttaataacct

SEQ ID NO: 211 - DNA, 32 bp, synthetic construct
  source 1..32: mol_type = other DNA
gaaattgtccaggcaaagcgccattccagcca

SEQ ID NO: 212 - DNA, 32 bp, synthetic construct
  source 1..32: mol_type = other DNA
cagatgaatcgcctgataaattgtaagacttt

SEQ ID NO: 213 - DNA, 32 bp, synthetic construct
  source 1..32: mol_type = other DNA
ttattacatacataacgccaaaagaatgttta

SEQ ID NO: 214 - DNA, 32 bp, synthetic construct
  source 1..32: mol_type = other DNA
ttcgtaatgcaactgttgggaaggcgacgaca

SEQ ID NO: 215 - DNA, 19 bp, synthetic construct
  source 1..19: mol_type = other DNA
gctttccggcaccgcttct

SEQ ID NO: 216 - DNA, 32 bp, synthetic construct
  source 1..32: mol_type = other DNA
gatcgcactcgccattcaggctgccatggtca

SEQ ID NO: 217 - DNA, 30 bp, synthetic construct
  source 1..30: mol_type = other DNA
aatgaaaagacaaaaggattggcagattca

SEQ ID NO: 218 - DNA, 30 bp, synthetic construct
  source 1..30: mol_type = other DNA
aagagaatcgatgccaaatcaacgagttga

SEQ ID NO: 219 - DNA, 32 bp, synthetic construct
  source 1..32: mol_type = other DNA
gataatcatttcatcaacattaaatggtgtag

SEQ ID NO: 220 - DNA, 32 bp, synthetic construct
  source 1..32: mol_type = other DNA
agtagtaaagaaaaatctacgttataagagca

SEQ ID NO: 221 - DNA, 32 bp, synthetic construct
  source 1..32: mol_type = other DNA
acaataacattcctgattatcagagtaacatt

SEQ ID NO: 222 - DNA, 32 bp, synthetic construct
  source 1..32: mol_type = other DNA
agcccaatagcgtctttccagagccagctaca

SEQ ID NO: 223 - DNA, 32 bp, synthetic construct
  source 1..32: mol_type = other DNA
caaggcgaatgggataggtcacgttgtgagcg

SEQ ID NO: 224 - DNA, 32 bp, synthetic construct
  source 1..32: mol_type = other DNA
tgcttctgcagaggcgaattattccgggagaa

SEQ ID NO: 225 - DNA, 32 bp, synthetic construct
  source 1..32: mol_type = other DNA
ttgctttgaaagcactaaatcggaggaacaag

SEQ ID NO: 226 - DNA, 32 bp, synthetic construct
  source 1..32: mol_type = other DNA
ataataacataaaggtggcaacattgggaatt

SEQ ID NO: 227 - DNA, 32 bp, synthetic construct
  source 1..32: mol_type = other DNA
ttgtatcacggtgtacagaccaggccagaacg

SEQ ID NO: 228 - DNA, 32 bp, synthetic construct
  source 1..32: mol_type = other DNA
atcctaataggtaaagtaattctgagtagggc

SEQ ID NO: 229 - DNA, 20 bp, synthetic construct
  source 1..20: mol_type = other DNA
agactagactagactagact

SEQ ID NO: 230 - DNA, 15 bp, synthetic construct
  source 1..15: mol_type = other DNA
ccccccccccccccc

(19) United States
(12) Patent Application Publication
(10) Pub. No.: US 2026/0308666 A1
(43) Pub. Date: Oct. 8, 2026

(54) Nucleic Acid Scaffolds and Related Devices and Methods

(71) Applicant: Digital Biotechnologies, Inc., Seattle, WA (US)

(72) Inventor: Jason H. Bielas, Seattle, WA (US)

(21) Appl. No.: 19/479,219

(22) PCT Filed: May 10, 2024
(86) PCT No.: PCT/US2024/028904

§ 371 Date: Oct. 27, 2025

Related U.S. Application Data
(60) Provisional application No. 63/465,790, filed on May 11, 2023.

(51) Int. Cl. C12Q 1/6825; G01N 33/487

(52) U.S. Cl. CPC C12Q 1/6825; G01N 33/48721

(57) ABSTRACT

Provided are nucleic acid scaffolds. In some embodiments, the nucleic acid scaffolds comprise a structure having a fixed or variable resistance operably coupled to site-specifically metalized nucleic acid regions within the nucleic acid scaffold. The structure having a fixed or variable resistance comprises an opening formed by one or more non-metalized nucleic acid regions of the nucleic acid scaffold, and when in use, conductance between two or more of the site-specifically metalized nucleic acid regions is conditioned upon a state of the structure having fixed or variable resistance. In some instances, the state of the structure is the presence or absence of an analyte within the opening, e.g., the presence or absence of a polymer (e.g., a nucleic acid or protein) translocating through the opening. Also provided are devices and methods that employ the nucleic acid scaffolds of the present disclosure, e.g., for detection and/or analysis of analytes of interest.

### CROSS-REFERENCE TO RELATED APPLICATION

This application claims the benefit of U.S. Provisional Patent Application No. 63/465,790, filed May 11, 2023, which application is incorporated herein by reference in its entirety.

### SUMMARY

Provided are nucleic acid scaffolds. In some embodiments, the nucleic acid scaffolds comprise a structure having a fixed or variable resistance operably coupled to site-specifically metalized nucleic acid regions within the nucleic acid scaffold. The structure having a fixed or variable resistance comprises an opening formed by one or more non-metalized nucleic acid regions of the nucleic acid scaffold, and when in use, conductance between two or more of the site-specifically metalized nucleic acid regions is conditioned upon a state of the structure having fixed or variable resistance. In some instances, the state of the structure is the presence or absence of an analyte within the opening, e.g., the presence or absence of a polymer (e.g., a nucleic acid or protein) translocating through the opening. Also provided are devices and methods that employ the nucleic acid scaffolds of the present disclosure, e.g., for detection and/or analysis of analytes of interest.

### BRIEF DESCRIPTION OF THE FIGURES

FIG. 1A-1C: A schematic illustration of a nucleic acid scaffold operably coupled to electrodes according to embodiments of the present disclosure.

FIG. 2A-2B: Schematic illustrations of site-specifically metalized nucleic acid scaffolds according to embodiments of the present disclosure.

FIG. 3A-3D: Schematic illustrations of additional site-specifically metalized nucleic acid scaffolds operably coupled to electrodes according to embodiments of the present disclosure.

FIG. 4: Schematic illustration of an embodiment of the present disclosure in which a site-specifically metalized nucleic acid scaffold is inserted in a nanopore.

FIG. 5A-5B: Schematic illustrations of non-limiting approach for site-specific metallization of nucleic acid scaffolds.

FIG. 6A-6B: Depiction of a nucleic acid scaffold with a 4 nm pore.

FIG. 7A-7B: A further depiction of the nucleic acid scaffold with a 4 nm pore shown in FIG. 6A-6B.

FIG. 8A-8B: Images of the triangle design—outlining the scaffold and staple DNA with protruding oligo density field.

FIG. 9A-9B: TEM images demonstrating selective metallization (dark contrast) of triangles.

FIG. 10: An atomic force microscopy (AFM) image of thiolated DNA origami triangles selectively bound to gold electrodes lithographically patterned on silicon nitride.

FIG. 11A-11C: AFM images showing selective binding of thiolated triangle origami to Au after sonication added to chip cleaning process.

FIG. 12A-12C: AFM images showing selective binding of thiolated triangle origami to Au with gentler washing post-deposition.

FIG. 13A-13C: AFM images showing selective binding of thiolated triangle origami to Au with UV ozone cleaning.

FIG. 14A-14B: Reference triangle origami AFM images.

FIG. 15A-15B: Reference triangle origami AFM images.

FIG. 16A-16B: TEM images of dimers and clusters of DNA origami triangles joined by cytosine-cytosine mismatches mediated by silver ions in overhanging, single-stranded poly-cytosine oligonucleotides.

FIG. 17: A device according to embodiments of the present disclosure.

### DETAILED DESCRIPTION

Before the scaffolds, devices and methods of the present disclosure are described in greater detail, it is to be understood that the scaffolds, devices and methods are not limited to particular embodiments described, as such may, of course, vary. It is also to be understood that the terminology used herein is for the purpose of describing particular embodiments only, and is not intended to be limiting, since the scope of the scaffolds, devices and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the scaffolds, devices and methods. The upper and lower limits of these smaller ranges may independently be included in the smaller ranges and are also encompassed within the scaffolds, devices and methods, subject to any specifically excluded limit in the stated range. Where the stated range includes one or both of the limits, ranges excluding either or both of those included limits are also included in the scaffolds, devices and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the scaffolds, devices and methods belong. Although any scaffolds, devices and methods similar or equivalent to those described herein can also be used in the practice or testing of the scaffolds, devices and methods, representative illustrative scaffolds, devices and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present scaffolds, devices and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the scaffolds, devices and methods, which are, for clarity, described in the context of separate embodiments, may also be provided in combination in a single embodiment. Conversely, various features of the scaffolds, devices and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present scaffolds, devices and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Definitions

The terms “nucleic acid”, “polynucleotide” and “oligonucleotide” (or “oligo”) are used interchangeably herein to describe a polymer of any length, e.g., greater than about 2 bases, greater than about 10 bases, greater than about 100 bases, greater than about 500 bases, greater than 1000 bases, greater than 10,000 bases, greater than 100,000 bases, greater than about 1,000,000, up to about 10¹⁰ or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally occurring nucleotides include guanine, cytosine, adenine, thymine, and uracil (G, C, A, T and U respectively). DNA and RNA have a deoxyribose and ribose sugar backbone, respectively, whereas PNA's backbone is composed of repeating N-(2-aminoethyl)-glycine units linked by peptide bonds. In PNA various purine and pyrimidine bases are linked to the backbone by methylenecarbonyl bonds. A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The term “unstructured nucleic acid,” or “UNA,” is a nucleic acid containing non-natural nucleotides that bind to each other with reduced stability. For example, an unstructured nucleic acid may contain a G′ residue and a C′ residue, where these residues correspond to non-naturally occurring forms, i.e., analogs, of G and C that base pair with each other with reduced stability but retain an ability to base pair with naturally occurring C and G residues, respectively. Unstructured nucleic acid is described in US20050233340, which is incorporated by reference herein for disclosure of UNA.

In some embodiments, an “oligonucleotide” has a length of from 5-200 nucleotides, e.g., from 10-100 nucleotides.

According to some embodiments, the nucleic acid scaffolds of the present disclosure comprise one or more thermostability-increasing nucleotides, non-limiting examples of which include 2-Amino-2′-deoxyadenosine-5′-Triphosphate (2-Amino-dATP), 5-Methyl-2′-deoxycytidine-5′-Triphosphate (5-Me-dCTP), 5-Propynyl-2′-deoxycytidine-5′-Triphosphate (5-Pr-dCTP), 5-Propynyl-2′-deoxyuridine-5′-Triphosphate (5-Pr-dUTP) and/or halogenated deoxy-uridine (XdU) such as 5-Chloro-2′-deoxyuridine-5′-Triphosphate (5-CI-dUTP), 5-Bromo-2′-deoxyuridine-5′-Triphosphate (5-Br-dUTP), or any combination thereof.

The terms “complementary” or “complementarity” as used herein refer to a nucleotide sequence of a first nucleic acid that base-pairs by non-covalent bonds to a region of a second nucleic acid, or a nucleotide sequence of a first region of a nucleic acid that base-pairs by non-covalent bonds to a second region of the nucleic acid (e.g., a stem region). In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. In RNA, A is complementary to U and vice versa. Typically, “complementary” or “complementarity” refers to a nucleotide sequence that is at least partially complementary. These terms may also encompass duplexes that are fully complementary such that every nucleotide in one strand is complementary to every nucleotide in the other strand in corresponding positions. In certain cases, a nucleotide sequence may be partially complementary to a target, in which not all nucleotides are complementary to every nucleotide in the target nucleic acid in all the corresponding positions. For example, a region of a first nucleic acid may be perfectly (i.e., 100%) complementary to a region of a second nucleic acid, or the region of the first nucleic acid may share some degree of complementarity which is less than perfect (e.g., 70%, 75%, 85%, 90%, 95%, 99%). The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=# of identical positions/total # of positions ×100). When a position in one sequence is occupied by the same nucleotide as the corresponding position in the other sequence, then the molecules are identical at that position. A non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In some embodiments, parameters for sequence comparison can be set at score=100, wordlength=12, or can be varied (e.g., wordlength=5 or wordlength=20).

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones.

As used herein, “conductance” refers to the flow of electrical charge which may be influenced and defined by the impendence, resistance, and/or reactance within a circuit.

### Nucleic Acid Scaffolds

The present disclosure provides nucleic acid scaffolds (sometimes referred to herein as “origami”). In certain embodiments, the nucleic acid scaffolds comprise a structure having a fixed or variable resistance operably coupled to site-specifically metalized nucleic acid regions within the nucleic acid scaffold. The structure having a fixed or variable resistance comprises an opening formed by one or more non-metalized nucleic acid regions of the nucleic acid scaffold, and when in use, conductance between two or more of the site-specifically metalized nucleic acid regions is conditioned upon a state of the structure having fixed or variable resistance. In some instances, the state of the structure is the presence or absence of an analyte within the opening, e.g., the presence or absence of a polymer (e.g., a nucleic acid or protein) translocating through the opening.

The nucleic acid scaffold can be any convenient nucleic acid configured in such a manner that it at least partially forms an opening. In embodiments, the nucleic acid scaffold includes at least a nucleobase that base pairs with adenine, a nucleobase that base pairs with thymine or uracil, a nucleobase that base pairs with guanine, and a nucleobase that base pairs with cytosine. In certain cases, the nucleic acid scaffold includes one or more abasic nucleotides. The nucleic acid scaffold may, in some cases, be a single stranded nucleic acid. The nucleic acid scaffold may in some cases be electrolyzed. As discussed herein, an “electrolyzed” nucleic acid refers to nucleic acid through which an electric current is being passed. Without being bound by theory, it is believed that DNA backbones can support multiple charge transfer mechanisms that arise from the small activation gaps induced by water and counterions. Any suitable number of nucleic acids may be employed in the scaffold. In some cases, the number of nucleic acids ranges from 1 to 50, such as 1 to 15, and including 1 to 10. In some embodiments, scaffolds of the invention include 2 or more nucleic acids. In still other embodiments, scaffolds of the invention include 3 or more electrolyzed nucleic acids. Each of the plurality of nucleic acids may adopt the same configuration, or different configurations.

The structure comprising the opening separates two or more site-specifically metalized nucleic acid regions, e.g., separated first and second site-specifically metalized nucleic acid regions. Metallization is discussed herein in its conventional sense to describe the application of a metal to the nucleic acid. For example, the conductivity of the nucleic acid can be modified with dopants such as conductive metal nanoparticles. Metal nanoparticles of interest include, but are not limited to, gold, lead sulfide, lead selenide, germanium, and silver. In some embodiments, the conductivity of the nucleic acid can be modified with dopants such as conductive carbon, such as carbon nanotubes, carbon nanorods, carbon black, graphene sheets, graphene nanoribbons, and carbon nanofibers. In additional cases, the conductivity of regions of the scaffold is modified by molecular doping with intercalators such as anthraquinone, ferrocene, norbornadiene, methylene blue, ethidium, coralyne and cryptolepin. Nucleic acid intercalation involves the insertion of one intercalating moiety (mono-intercalator), two intercalating moieties (bis-intercalator) or multiple intercalating moieties into the nucleic acid structure. Intercalation changes the conduction/resistance of the complex influencing the overall conduction characteristics of the nucleic acid strand. In one embodiment, the dopant can be reduced or oxidized by the applied current.

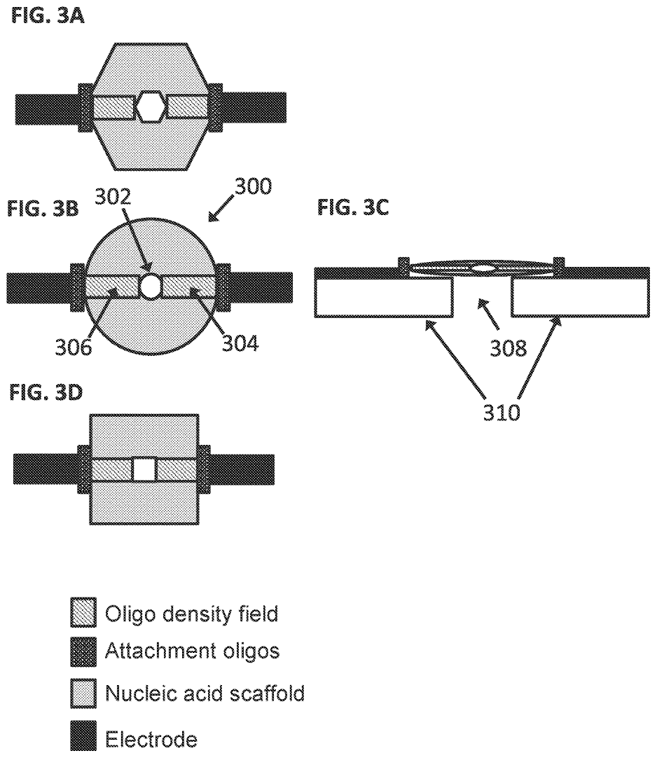

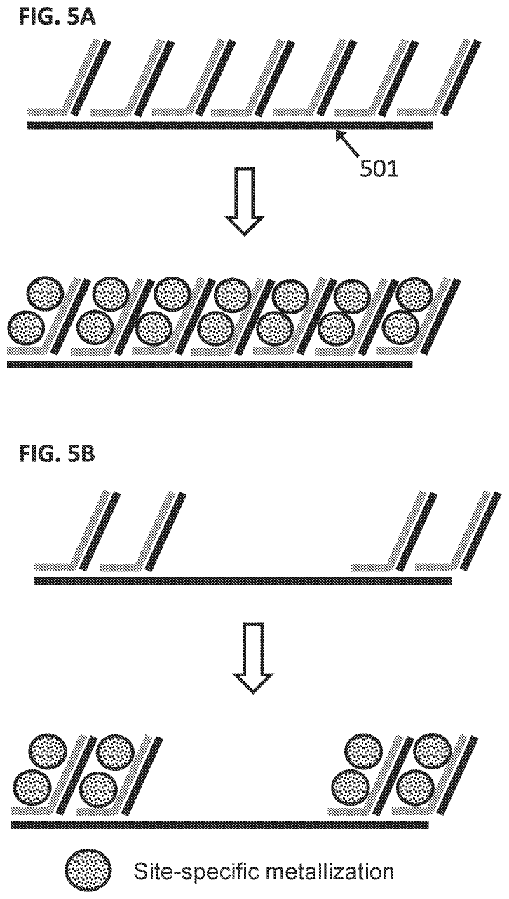

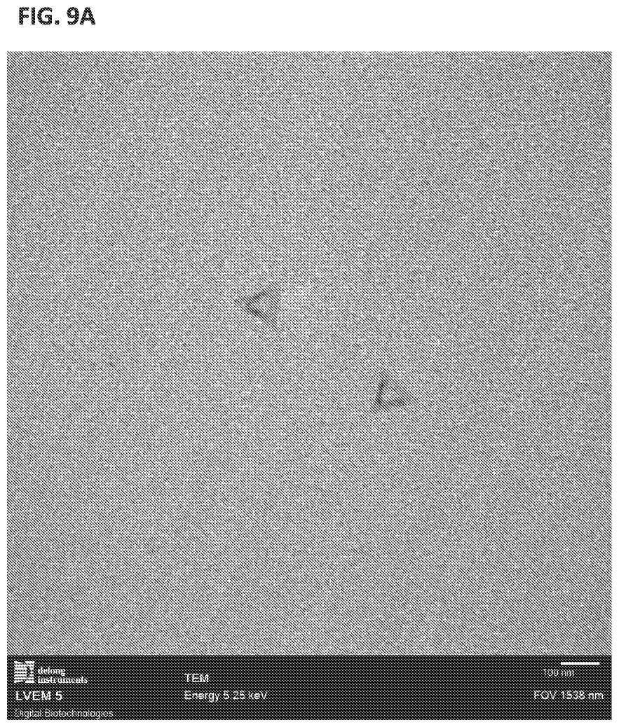

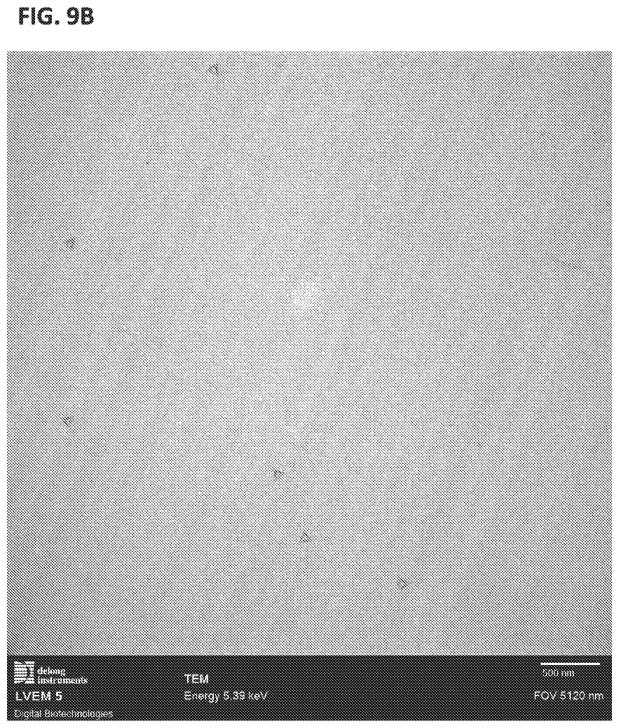

As discussed herein, “site specific” metallization refers to the metallization of only certain regions of the nucleic acid (or a field of nucleic acids—e.g., as in FIG. 3, FIG. 5, FIG. 9, etc.) while others are left non-metallized. As will be appreciated, the term “metalized region” or “metalized nucleic acid region” does not require complete metallization, and similarly, the term “non-metalized region” or “non-metalized nucleic acid region” does not require a complete absence of metallization, and such is not required for the scaffolds and devices of the present disclosure to perform as intended. In some cases, a nucleic acid of the disclosure may be considered site-specifically metallized if 5% or less of unintended sites (i.e., those not corresponding the regions of interest) are metallized, such as 4% or less, such as 3% or less, such as 2% or less, such as 1% or less and including 0%. In embodiments, the metallized sites are determined by nucleic acid density. In some such embodiments, the metallized sites are determined based on a number of nucleic acid strands. For example, in some versions, the metallization conditions are such that density-rich nucleic acid duplexes are preferentially metalized as compared to single-stranded nucleic acid regions. In some cases, regions of the nucleic acid scaffold comprise protruding oligos, such as double-stranded oligos, that increase nucleic acid density, e.g., for metallization. The length of the oligos may vary, and in some cases range from 10 bp to 50 bp, such as 15 bp to 25 bp, and including 18 bp to 22 bp. Modification of the length or density of these oligos could allow for discriminatory metallization, due to the preference of longer, denser oligo arrays for metal ions and the conformational differences of double- and single-stranded DNA in solution. See Dai et al. (2022) DNA Origami-Encoded Integration of Heterostructures. Angew. Chem. Int. Ed. 61(11):e202114190. doi: 10.1002/anie.202114190. Epub 2022 Jan. 27. PMID: 34962699. Chemistries used for site-specific metallization may depend based on the nature of the metal.

The nucleic acid scaffolds of the present disclosure utilize the base pairing properties of nucleic acids which enable the modular design and self-assembly of a scaffold comprising a nanometer-scale opening (or in any embodiments, a “gap”, “space” or “gate”) with a precisely defined size and shape. For example, the sequences and lengths of the nucleic acids may be selected to provide a scaffold having an opening of a selected nanometer-scale size for use as a detection region for one or more analytes of interest, e.g., small molecules, nucleic acids, proteins, or the like. The structure having a fixed or variable resistance comprises an opening formed by one or more non-metalized nucleic acid regions of the nucleic acid scaffold. In certain embodiments, the largest dimension of the opening is from 1 to 1000 nanometer (nm), e.g., from 1 to 750 nm, from 1 to 500 nm, from 1 to 250 nm, or from 1 to 100 nm. In certain embodiments, the opening has a largest dimension of from 2 to 20 nm, from 2 to 15 nm, or from 2 to 10 nm, e.g., from 2 to 5 nm.

As discussed above, the structure having a fixed or variable resistance comprises an opening formed by one or more non-metalized nucleic acid regions of the nucleic acid scaffold. Put another way, the regions of the nucleic acid forming the opening are not metallized, or are metallized to a negligible extent. In select versions, the one or more non-metalized nucleic acid regions forming the opening share one or more nucleic acids with the two or more site-specifically metalized nucleic acid regions. In other words, the metallized and non-metallized regions need not be entirely different strands. Instead, these regions can exist on one or more strands of the scaffold. As discussed above, nucleic acid density (e.g., whether a given strand exists as a double stranded polynucleotide) may at least partially determine metallization. In certain versions, the one or more non-metalized nucleic acid regions forming the opening comprise a nucleic acid hybridized to a nucleic acid of the two or more site-specifically metalized nucleic acid regions. In other words, the one or more non-metalized nucleic acid regions forming the opening are distinct strands relative to the strands comprising the one or more metallized regions, but are connected.

In some cases, the nucleic acid scaffold is attached to a surface. The nucleic acid may be stably associated with the surface in any suitable manner. By “stably associated”, it is meant that the nucleic acid scaffold does not readily dissociate from the surface. In certain cases, the nucleic acid scaffold comprises first and second discontinuous regions attached to a surface. By “discontinuous” regions, it is meant that the first and second regions are not the same regions (i.e., they do not overlap). The discontinuous regions of the nucleic acid scaffold may be attached to the same or different surfaces. Each surface may be comprised of any convenient material. In certain cases, the surface is a metal surface (e.g., a gold surface). In some embodiments, the nucleic acid scaffold is stably associated with the surface via thiol bonding chemistry. For example, in embodiments where the surface is a gold surface, thiols may directly react with the gold surface to form Au—S bonds via an oxidation-reduction reaction. In additional embodiments, stably associating the scaffold with the surface comprises dip pen nanolithography. In such embodiments, an atomic force microscope may be employed to imprint thiolates onto the surfaces. Dip pen nanolithography is described in, e.g., U.S. Pat. Nos. 8,261,662 and 9,403,180; the disclosures of which are herein incorporated by reference in their entirety. In other embodiments, the nucleic acid scaffold is stably associated with the surface via a biotin-streptavidin interaction. In select cases, the nucleic acid scaffold is in contact with multiple surfaces. In other words, one end of the nucleic acid scaffold may be in contact with a first surface, and the other end of the nucleic acid scaffold may be in contact with a second surface that is distinct from the first surface.

In select cases, the nucleic acid scaffold is attached by coating the surface with a biocompatible layer. The biocompatible layer may vary and can include, e.g., a polymer matrix, gel, or self-assembled monolayer (SAM). In certain embodiments, an alkanethiol SAM is produced on one or more of the surfaces. In such cases, the surface (e.g., gold surface) is incubated with a thiol-functionalized blocking molecule. Thiol-functionalized blocking molecules include, but are not limited to 1-mercapto-11-undecanol, 1-mercapto-6-hexanol, hexadecanethiol, combinations thereof and the like. SAM formation is described in, e.g., Szymonik et al. Nanotechnology, 27(39), 395301; herein incorporated by reference in its entirety. Coating may include applying a negative voltage to the surface in solution, thereby causing the thiol bond to break and releasing the blocking molecule. Suitable methods for such electrochemical desorption may be found in, e.g., Widrig et al. Journal of electroanalytical chemistry and interfacial electrochemistry, 310(1-2), 335-359; herein incorporated by reference in its entirety. After the release of the blocking molecule, the attachment can involve incubating a mixture of 5′- or 3′-thiol functionalized nucleic acid (e.g., oligonucleotides) of interest and a thiol-containing blocking molecule to form a new electrode-bound monolayer interspersed with the nucleic acid of interest. Methods that may be adopted for use in surface functionalization are described in, e.g., Walti et al. Langmuir, 19(4), 981-984; herein incorporated by reference in its entirety.

In some cases, the surface(s) to which the nucleic acid scaffold is bound is an electrode. Put another way, in order to pass the current through the at least a portion of the scaffold, the 5′ and/or 3′ end of the nucleic acid may in some cases be in contact with at least one electrode. In select versions, one end of the scaffold is stably associated with an electrode, while the other end is stably associated with a non-electrode surface. In other cases, both ends of the scaffold are stably associated with different electrodes. The electrode(s) may be comprised of any convenient material. In some embodiments, electrodes are metal electrodes. Materials for use in metal electrodes include, but are not limited to platinum, gold, titanium nitride, silver, and graphite. In certain embodiments, the electrodes are gold electrodes.

In select embodiments, the surface (e.g., electrode surface) is functionalized with oligonucleotides comprising sequences complementary to the first and second discontinuous regions and the first and second discontinuous regions are attached to the surface via hybridization to the oligonucleotides The oligonucleotide(s) may be any suitable short (e.g., 5-100 nucleotides) single-stranded DNA or RNA molecule. In certain cases, each oligonucleotide has a sequence that is complementary to a sequence of the scaffold, i.e., such that the two molecules hybridize. Because the oligonucleotides are anchored to a surface (e.g., electrode surface), a nucleic acid scaffold hybridized to the oligonucleotides can be stably associated with the at least one surface.

In select embodiments, the two or more site-specifically metalized nucleic acid regions comprise a first metalized field adjacent the opening at a first location of the opening, and a second metalized field adjacent the opening at a second location of the opening. As discussed herein “metallized fields” (also referred to herein as “density fields”) are regions of the scaffold adjacent to the opening that are metallized in a site-specific manner (e.g., as discussed above). In certain cases, the first and second locations of the opening are opposite each other. In some versions, site-specifically metalized oligo density fields are operably coupled to electrodes (e.g., such as those described above). in some such cases, the site-specifically metalized fields are operably coupled to the electrodes via attachment nucleic acids (e.g., “attachment oligos”). In select instances, the attachment nucleic acids are also metalized. The electrodes may in turn be operatively coupled to a power source, enabling the application of a bias between the site-specifically metalized oligo density fields.

A sample (e.g., a biological sample) may be interrogated for the presence or concentration of one or more analytes of interest, and/or the identities of monomers within a polymer (including a sequence thereof) may be determined, based on qualitative or quantitative changes and/or variations in the conductance resulting from the presence of the analyte within (e.g., translocating through) the opening. The scaffolds enable improved analyte detection and/or analysis of analytes of interest, e.g., on account of the precise size of the opening (or “detection region”) achievable using the nucleic acid-based approach described herein.

When an electrical bias is provided between the two or more site-specifically metalized nucleic acid regions, the structure provides a fixed or variable resistance between the two or more site-specifically metalized nucleic acid regions. The resistance of the structure, and in turn, conductance between the two or more site-specifically metalized nucleic acid regions, depends upon the state of the structure, where the state may be the presence or absence of an analyte within (e.g., translocating through) the opening of the structure. In other words, that which causes the fixed or variable resistance is the presence of the analyte. In some such embodiments where the analyte is a polymer (e.g., nucleic acid, protein), the state of the structure having fixed or variable resistance is the presence or absence of a polymer translocating through the opening. In a particular embodiment, conductance between the two or more site-specifically metalized nucleic acid regions varies, or occurs only, in the presence of a nucleic acid translocating through the opening. In select cases, conductance between the two or more site-specifically metalized nucleic acid regions increases in the presence of the analyte translocating through the opening. For example, the scaffold may be configured for detection of a varying conductance indicative of sequential interactions with an analyte (e.g., nucleic acid or protein being sequenced). Without being bound by theory, it is understood that interactions between given nucleobases of nucleic acids are characterized by a particular conductance fingerprint. In other words, the DNA base pair behaves as a biological Aviram-Ratner electrical rectifier because of the spatial separation and weak bonding between the nucleobases, and because the current that flow across these base pairs varies based on the specific nature of the bond. In an exemplary application of the claimed scaffold, such conductance fingerprints may be identified as an analyte nucleic acid passes through the opening thereby identifying characteristics of the nucleic acid including, but not limited to, the sequence of the nucleic acid. Additional details regarding conductance fingerprints may be found in International Application Publication No. WO 2023/086416; the disclosure of which is incorporated by reference herein. In embodiments, an interaction between the base to be sequenced and the scaffold bases will lead to a varying conductance fingerprint which will allow for the identification of the sequenced base. Measurement of the electrons traveling across the nucleic acids allows for the assessment of said conductance fingerprint. The conductance along the analyte may be measured via any convenient approach. In some embodiments, an electrometer is employed to provide a bias current and analyze the resultant current. Commercially available electrometers that may be suitable for use in the subject methods include, e.g., Keithley® instruments. In some embodiments, detecting the varying conductance includes an impedance-based approach. In certain versions, methods include identifying a nucleobase based its characteristic energy levels. Characteristic energy levels of interest include, but are not limited to, highest occupied molecular orbital (HOMO) energy. Measurement techniques that may be adapted for use in the subject methods can be found in, e.g., Pedersen et al. Nanotechnology, 28(1), 015502; and Ohshiro et al. 2012 12th IEEE International Conference on Nanotechnology (IEEE-NANO) (pp. 1-2). IEEE; herein incorporated by reference in their entirety. Details regarding embodiments of the scaffolds of the present disclosure will now be described.

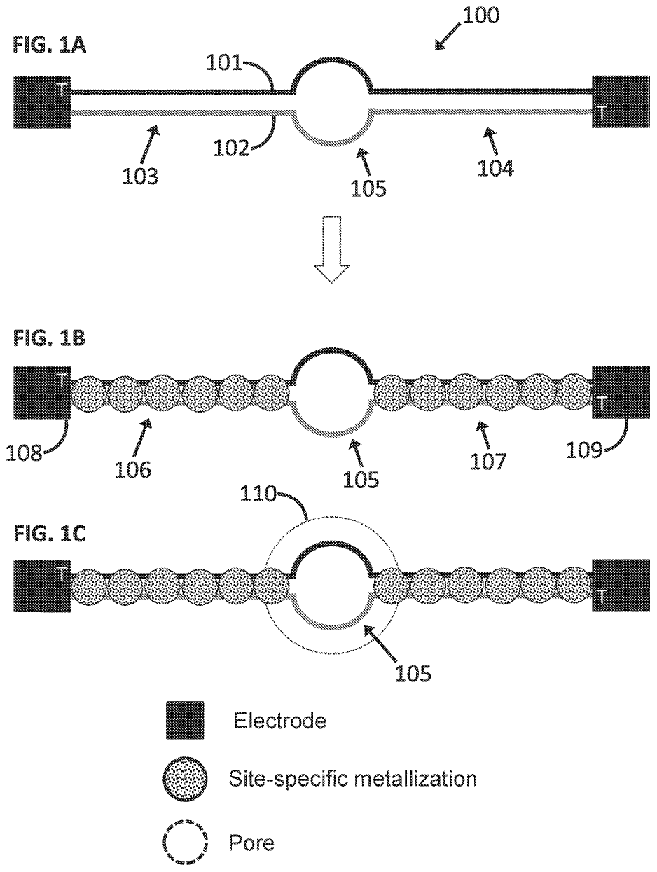

A non-limiting example of a nucleic acid scaffold of the present disclosure is schematically illustrated in FIG. 1. In this example, nucleic acid scaffold 100 comprises first nucleic acid 101 and second nucleic acid 102. The sequences of first nucleic acid 101 and second nucleic acid 102 are designed to provide first stem region 103, second stem region 104, and a “loop” structure 105. FIG. 1B shows the site-specifically metalized form of the scaffold of FIG. 1A, where under the conditions in this example, double stranded DNA is preferentially metalized, resulting in site-specific metallization of the stem regions to produce first site-specifically metalized nucleic acid region 106 and second site-specifically metalized nucleic acid region 107. Thus, in this example, the non-metalized nucleic acid region forming the opening shares two nucleic acids with the first and second site-specifically metalized nucleic acid regions.

In FIG. 1, the first and second site-specifically metalized nucleic acid regions are operably coupled to electrodes 108 and 109, respectively. Upon application of an electrical bias between the first and second site-specifically metalized nucleic acid regions, structure 105, comprising an opening formed by the non-metalized nucleic acid regions of first and second nucleic acids 101 and 102, has a fixed or variable resistance, where conductance between the first and second site-specifically metalized nucleic acid regions is conditioned upon a state of structure 105, e.g., the presence or absence of an analyte within or translocating through the opening.

FIG. 1C shows the site-specifically metalized scaffold of FIG. 1B in the context of a device comprising nanopore 110, where the nanopore is aligned with the opening of structure 105. The device may be adapted to translocate an analyte through the nanopore, thereby translocating the analyte through the opening. In this way, the nanopore finds use in facilitating translocation of an analyte through the opening (for purposes of detecting, quantifying, determining a sequence of, and/or the like, the analyte). For example, the nanopore may provide the sole passage through which an ionic solution on a first side of a membrane contacts the ionic solution on the second side of the membrane. A constant voltage bias may be employed to produce an ionic current through the nanopore that drives an analyte (e.g., a negatively charged analyte such as DNA or RNA) initially present in a chamber on the first side through the pore to a chamber on the second side.

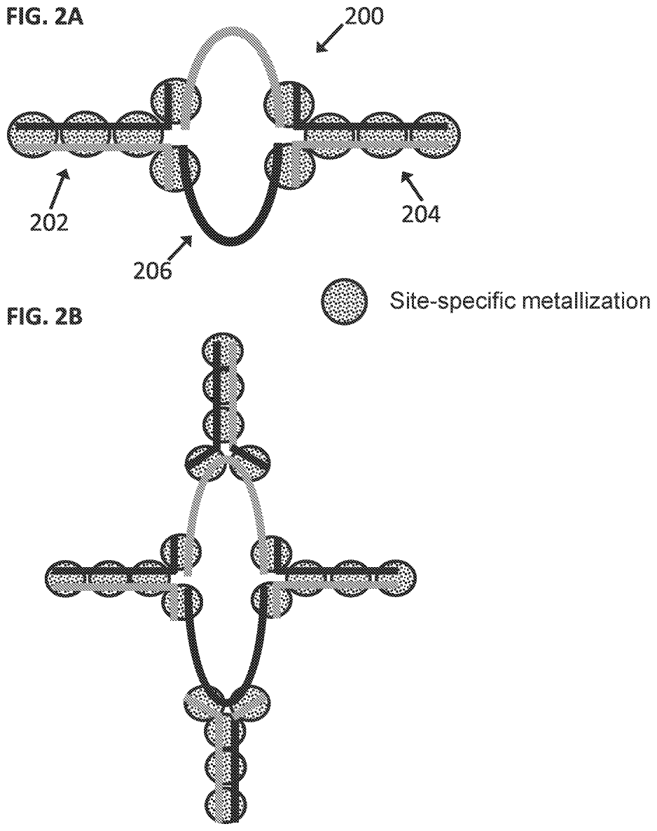

Additional non-limiting examples of site-specifically metalized nucleic acid scaffolds of the present disclosure are schematically illustrated in FIGS. 2A and 2B. Shown in FIG. 2A is scaffold 200 comprising site-specifically metalized regions 202 and 204 separated by structure 206 comprising an opening formed by non-metalized nucleic acid regions. In this example, the non-metalized nucleic acid regions comprise first and second nucleic acids designed to associate with the site-specifically metalized regions via hybridization. FIG. 2B shows a configuration in which four site-specifically metalized regions are separated by a structure comprising an opening formed by non-metalized nucleic acid regions.

Further non-limiting examples of site-specifically metalized nucleic acid scaffolds in the context of a device employing the same are schematically illustrated in FIG. 3. In these examples, site-specifically metalized nucleic acid density fields (here, oligonucleotide (or “oligo”) density fields) are disposed adjacent the opening at different locations. With reference to FIG. 3B, scaffold 300 comprises structure 302 (comprising an opening formed by non-metalized nucleic acid regions) and site-specifically metalized oligo density fields 304 and 306 adjacent the opening at first and second substantially opposite locations, respectively. The site-specifically metalized oligo density fields are operably coupled to electrodes. In this example, the site-specifically metalized oligo density fields are operably coupled to the electrodes via attachment nucleic acids (here, “attachment oligos”), which are also subsequently metalized. The electrodes may in turn be operatively coupled to a power source, enabling the application of a bias between the site-specifically metalized oligo density fields. The device may comprise a nanopore, where the opening may be aligned with the nanopore. For example, FIG. 3C shows a side view of the arrangement of FIG. 3B, showing alignment of the opening with nanopore 308 formed in membrane (or substrate) 310. The device may be adapted to translocate an analyte through the nanopore, thereby translocating the analyte through the opening, as described elsewhere herein.

As will be appreciated with the benefit of the present disclosure, a nucleic acid scaffold may comprise a single nucleic acid scaffold unit, or may comprise two or more nucleic acid scaffold subunits (e.g., scaffold monomers) operably connected to each other. For example, the nucleic acid scaffolds shown in FIG. 3 may each comprise a unitary nucleic acid scaffold or may be produced from two or more scaffold subunits. A non-limiting example approach for producing scaffolds comprising two or more subunits is described in detail in Example 4 below.

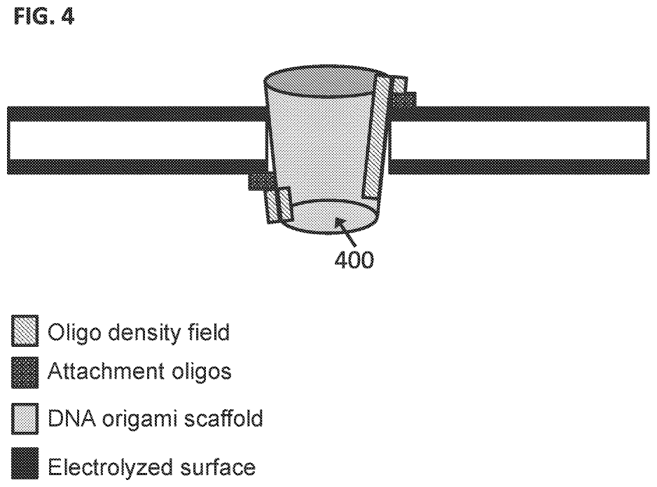

A further non-limiting example of a site-specifically metalized nucleic acid scaffold in the context of a device comprising a nanopore is schematically illustrated in FIG. 4. In this example, rather than being disposed over the nanopore, the site-specifically metalized nucleic acid scaffold is inserted in the nanopore. As shown, the nanopore is formed in a membrane or substrate comprising an electrolyzed surface on each side. The electrolyzed surfaces are operably coupled to site-specifically metalized oligo density fields of the scaffold via attachment oligos. Here, the scaffold has a three-dimensional shape which is substantially cylindrical or conical and sized for insertion into the nanopore. Opening 400 is formed by or substantially by non-metalized nucleic acids regions of the nucleic acid scaffold, where conductance between the oligo density fields is conditioned upon a state of the opening, e.g., the presence or absence of an analyte within (or translocating through) the opening.

Site-specific metallization of a nucleic acid scaffold may be achieved using a variety of approaches. One non-limiting example is schematically illustrated in FIG. 5. In this example, the metallization conditions are such that density-rich nucleic acid duplexes are preferentially metalized as compared to single-stranded nucleic acid regions. Shown in FIG. 5A is an example of how such duplexes may be provided in a scaffold. Here, “base” strand 501 to which oligonucleotides comprising a domain complementary to the base strand are hybridized. A third nucleic acid species is hybridized to the domain of the second species not hybridized to the base strand, thereby providing metallization targets within the scaffold (top). In FIG. 5A, the base strand is completely or substantially completely occupied by the metallization targets, such that upon metallization (bottom), the base strand does not include a non-metalized region. In contrast, and as shown in FIG. 5B, the nucleic acid duplexes may be discontinuous along the base strand (e.g., by designing a base strand with one or more domains comprising nucleotide sequences which are non-complementary with others employed in the scaffold, and/or by masking the one or more domains with proteins that bind thereto during the hybridization step), such that upon metallization, a site-specifically metalized portion of the scaffold is formed comprising first and second metalized regions separated by a non-metalized region.

According to one non-limiting approach for site-specific metallization, planar DNA origami triangles were designed with oligonucleotides that protrude from the plane. One side of the triangle has 20-base-pair, double-stranded protruding oligos at a density of 7.79×10⁻² strands/nm². Another side has 20-base, single-stranded protruding oligos at a density of 7.79×10⁻² strands/nm². The third side has no protruding oligos. Modification of the length or density of these oligos could allow for discriminatory metallization, due to the preference of longer, denser oligo arrays for metal ions and the conformational differences of double- and single-stranded DNA in solution. See Dai et al. (2022) DNA Origami-Encoded Integration of Heterostructures. Angew. Chem. Int. Ed. 61(11):e202114190. doi: 10.1002/anie.202114190. Epub 2022 Jan. 27. PMID: 34962699.

In certain embodiments, partial silver metallization is performed. According to one particular approach, 180 femtomoles of DNA origami triangles are combined with 1.25 μL freshly prepared Tollen's reagent, which is formed by the reaction of 20 μL of 147 mM AgNO₃ and 8 μL NH₄OH (just until solution turns clear), then the addition of 1.4 μL nuclease-free water. To this mixture, 1.25 μL 200 mM glucose is added as a reductant, for a total metallization reaction volume of 62.5 μL. The metallization is carried out in complete darkness at room temperature without shaking for 15 minutes. See Zhang et al. (2021) Prescribing Silver Chirality with DNA Origami. J. Am. Chem. Soc. 143 (23), 8639-8646.

In some instances, partial gold metallization is performed. For example, 180 femtomoles of DNA origami triangles may be combined with 1.25 μL of 300 mM H(AuCl₄) for a total volume of 61.25 μL. The reaction is incubated in complete darkness at room temperature without shaking for 3 hours. Then, 1.25 μL of 600 mM glucose are added as a reductant. See Dai et al. (supra).

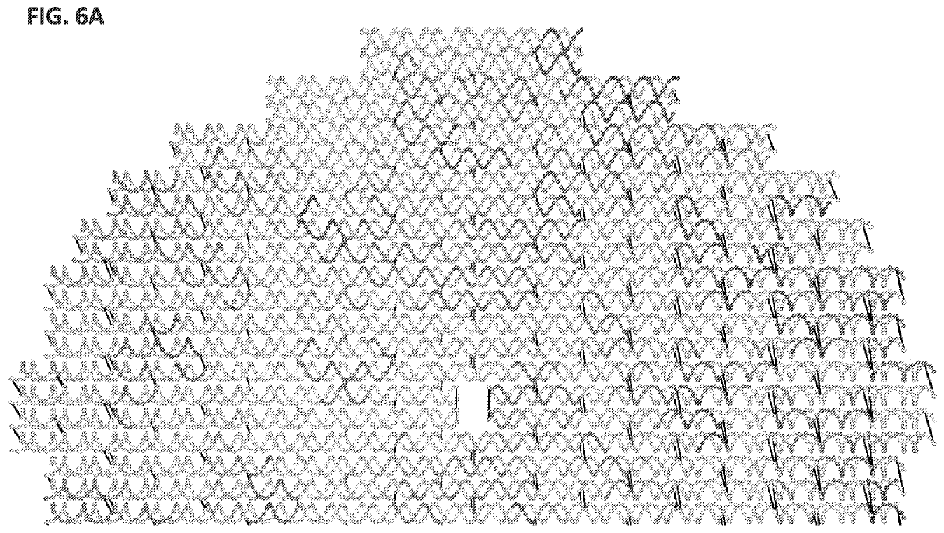

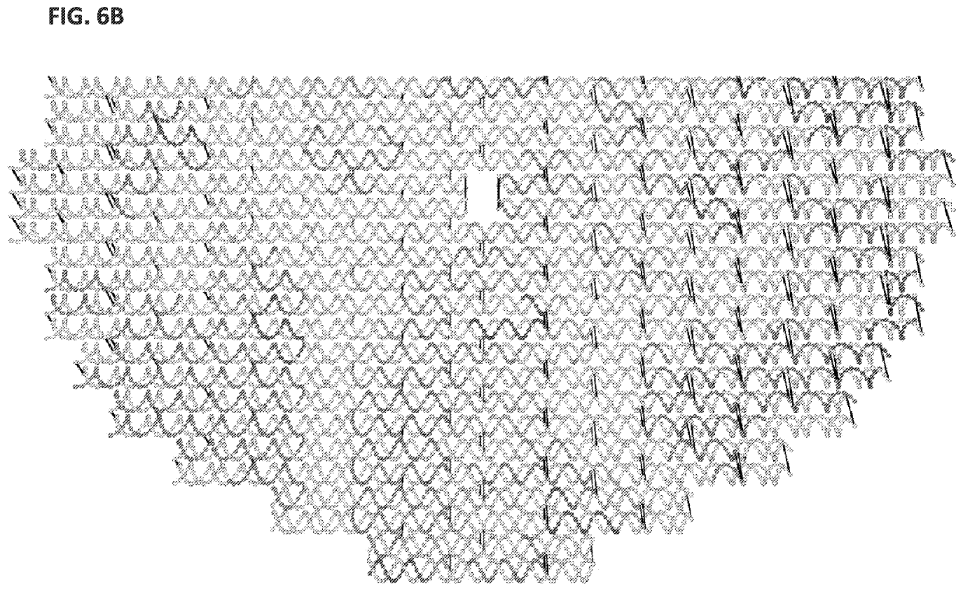

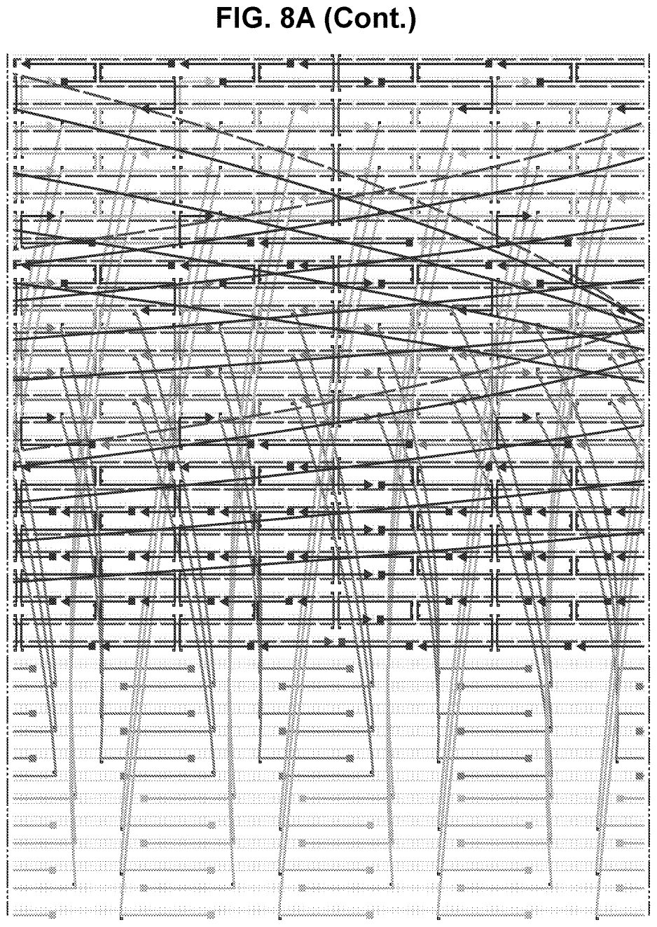

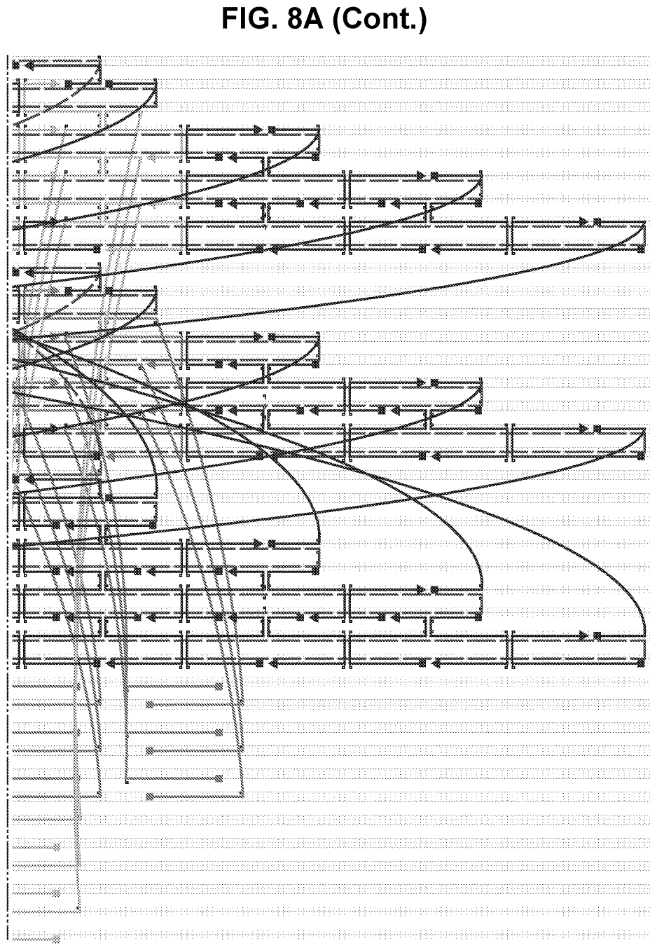

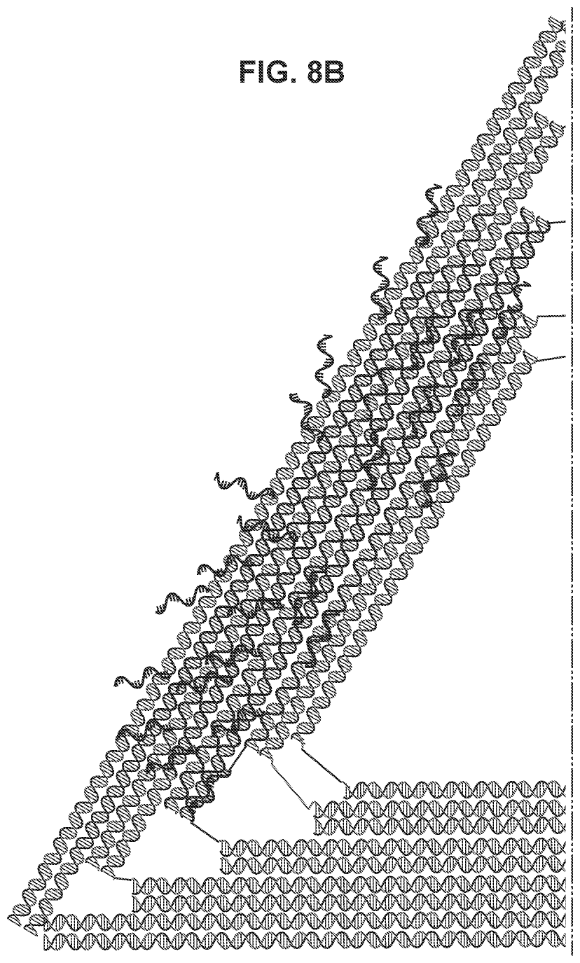

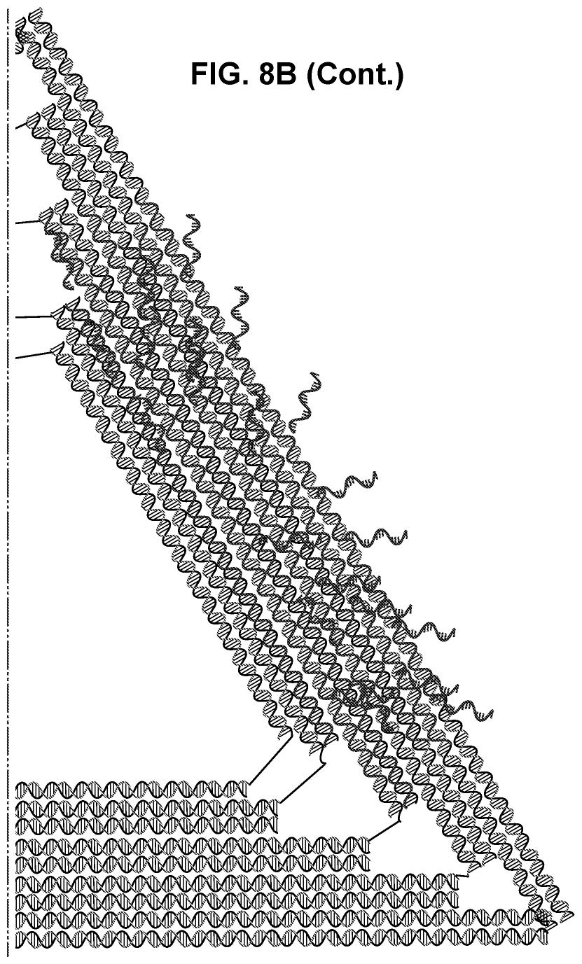

As will be appreciated with the benefit of the present disclosure, a nucleic acid scaffold of the present disclosure (e.g., such as those depicted in FIGS. 6-8) may comprise a single nucleic acid backbone or may comprise two or more nucleic acid backbones. In some instances, nucleic acid scaffolds comprising one or more nucleic acid backbones may be stabilized using “staple” oligonucleotides, e.g., as in FIGS. 6-8.

Conditions in which the scaffold may be employed may vary. In some cases, the scaffold is present in a vacuum. In other cases, the scaffold is immersed in a fluid. In some cases, the fluid is a gas. In other cases, the fluid is a liquid. In some cases, the fluid comprises an ionic concentration selected to achieve a desired conductance between the two or more site-specifically metalized nucleic acid regions. In embodiments, the conductance is a baseline conductance when an analyte is not present within the opening. For example, in embodiments, scaffolds are used in the presence of a suitable charge carrier, such as metal salts, for example alkali metal salts, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or I-ethyl-3-methyl imidazolium chloride. Generally, the salt is present in the aqueous solution in the chamber. Potassium chloride (KCl), sodium chloride (NaCl) or cesium chloride (CsCl) may be used, for example. The salt concentration may be at saturation. The salt concentration may be 3M or lower and is typically from 0.1 to 2.5 M, from 0.3 to 1.9 M, from 0.5 to 1.8 M, from 0.7 to 1.7 M, from 0.9 to 1.6 M, or from 1 M to 1.4 M. The salt concentration may be from 150 mM to 1 M. The methods are preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of a nucleotide to be identified against the background of normal current fluctuations.

Once the sequences, lengths, proportion of species, and/or the like of the nucleic acids are selected to produce a scaffold with the desired characteristics (e.g., opening size, opening shape, metallization targets (e.g., oligo density fields, and/or the like)), assembly of the scaffold is typically performed under conditions for specific hybridization. Whether specific hybridization occurs is determined by such factors as the degree of complementarity between the relevant nucleic acids, the length thereof, and the temperature at which the hybridization occurs, which may be informed by the melting temperatures (T_{M}) of the relevant portions of the nucleic acids. The melting temperature refers to the temperature at which half of the relevant nucleic acids remain hybridized and half of the relevant nucleic acids dissociate into single strands. The Tm of a duplex may be experimentally determined or predicted using the following formula Tm=81.5+16.6(log 10[Na⁺])+0.41 (fraction G+C)−(600/N), where N is the chain length and [Na⁺] is less than 1 M. See Sambrook and Russell (2001; Molecular Cloning: A Laboratory Manual, 3rd ed., Cold Spring Harbor Press, Cold Spring Harbor N.Y., Ch. 10). Other more advanced models that depend on various parameters may also be used to predict Tm of target capture nucleic acid/target nucleic acid duplexes depending on various hybridization conditions. Approaches for achieving specific nucleic acid hybridization may be found in, e.g., Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993).

### Devices and Computer Controlled Systems

Aspects of the present disclosure further include devices. In certain embodiments, a device of the present disclosure comprises any of the nucleic acid scaffolds of the present disclosure, electrodes operably coupled to the two or more site-specifically metalized nucleic acid regions, and one or more electric sources operably coupled to the electrodes. Such devices may further comprise one or more processors, and one or more computer-readable media comprising instructions stored thereon, which when executed by the one or more processors, cause the device to provide an electrical bias between the two or more site-specifically metalized nucleic acid regions, and measure a conductance between the two or more site-specifically metalized nucleic acid regions.

A variety of approaches may be employed to operably couple electrodes to the two or more site-specifically metalized nucleic acid regions. For example, in some embodiments, the electrodes are operably coupled to the two or more site-specifically metalized nucleic acid regions via attachment nucleic acids, which are subsequently metalized. The attachment nucleic acids may comprise docking nucleic acids immobilized on the surface of an electrode, where nucleic acids of the nucleic acid scaffold hybridize to the docking nucleic acids. The surface may be a metal surface (e.g., a gold surface). In some embodiments, the docking nucleic acids are stably associated with the surface via thiol bonding chemistry. For example, in embodiments where the surface is a gold surface, thiols may directly react with the gold surface to form Au—S bonds via an oxidation-reduction reaction.

The surface may be functionalized with any useful/convenient reactive group, including but not limited to thiol groups (—SH), amine groups (—NH₂), carboxyl groups (—COOH), and/or the like. Bioconjugation strategies that find use in binding any desirable components of the present disclosure are described in Hermanson, “Bioconjugate Techniques,” Academic Press, 2nd edition, Apr. 1, 2008, Haugland, 1995, Methods Mol. Biol. 45:205-21; Brinkley, 1992, Bioconjugate Chemistry 3:2, and elsewhere.

Functional groups that may be used to bind components include, but are not limited to, active esters, isocyanates, imidoesters, hydrazides, amino groups, aldehydes, ketones, photoreactive groups, maleimide groups, alpha-halo-acetyl groups, epoxides, azirdines, and the like. Reagents such as iodoacetamides, maleimides, benzylic halides and bromomethylketones react by S-alkylation of thiols to generate stable thioether products. For example, at pH 6.5-7.5, maleimide groups react with sulfhydryl groups to form stable thioether bonds. Arylating reagents such as NBD halides react with thiols or amines by a similar substitution of the aromatic halide by the nucleophile. Because the thiolate anion is a better nucleophile than the neutral thiol, cysteine is more reactive above its pKₐ (~8.3, depending on protein structural context). Thiols also react with certain amine-reactive reagents, including isothiocyanates and succinimidyl esters. The TS-Link series of reagents are available for reversible thiol modification.

With respect to amine reactive groups, primary amines exist at the N-terminus of polypeptide chains and in the side-chain of lysine (Lys, K) amino acid residues. Among the available functional groups in typical biological or protein samples, primary amines are especially nucleophilic, making them ready targets for conjugation with several reactive groups. For example, NHS esters are reactive groups formed by carbodiimide-activation of carboxylate molecules. NHS ester-activated crosslinkers and labeling compounds react with primary amines in physiologic to slightly alkaline conditions (pH 7.2 to 9) to yield stable amide bonds. The reaction releases N-hydroxysuccinimide (NHS). Also by way of example, imidoester crosslinkers react with primary amines to form amidine bonds. Imidoester crosslinkers react rapidly with amines at alkaline pH but have short half-lives. As the pH becomes more alkaline, the half-life and reactivity with amines increases. As such, crosslinking is more efficient when performed at pH 10 than at pH 8. Reaction conditions below pH 10 may result in side reactions, although amidine formation is favored between pH 8-10.

Numerous other synthetic chemical groups will form chemical bonds with primary amines, including but not limited to, isothiocyanates, isocyanates, acyl azides, sulfonyl chlorides, aldehydes, glyoxals, epoxides, oxiranes, carbonates, aryl halides, carbodiimides, anhydrides, and fluorophenyl esters. Such groups conjugate to amines by either acylation or alkylation.

Approaches for attachment also include click-chemistry based approaches. Click-chemistry reactions that may be employed include (i) nucleophilic substitutions; (ii) additions to C—C multiple bonds (e.g., Michael addition, epoxidation, dihydroxylation, aziridination); (iii) nonaldol like chemistry (e.g., N-hydroxysuccinimide active ester couplings); and (iv) cycloadditions (e.g., Diels-Adler reaction, Huisgen's cycloaddition). Huisgen's cycloaddition has been applied in various branches of chemistry. It consists of the condensation of organic azides with alkyne groups to form 1,2,3-triazole linkages. Azide and alkyne functionalities can be easily introduced in the scaffold of large organic constructs of biological relevance. The reaction may be catalyzed by introducing copper(I). The Cu(I) core has a dual effect in that it activates the slow-reacting alkyne group thus accelerating the azide-alkyne condensation kinetics by ~10⁷-10⁸-fold, and it organizes the reacting groups by “templation” so that only a regiospecific 1,4-disubstituted adduct is formed. This reaction is known as the copper-catalyzed azide alkyne cycloaddition (CuAAC), and its compatibility with a wide range of biological substrates and synthetic conditions makes CuAAC the flagship among click conjugations. Since its discovery, Cu(I)-catalyzed azide alkyne cycloaddition has been widely used within the fields of biology, biochemistry, and biotechnology. Click-chemistry reactions that may be employed to attach the nucleotide or polynucleotide to the particle include, but are not limited to, Huisgen Azide-Alkyne 1,3-Dipolar Cycloaddition, Copper-Catalyzed Azide-Alkyne Cycloaddition (CuAAC), Ruthenium-Catalyzed Azide-Alkyne Cycloaddition (RuAAC), and the like. Details regarding click-chemistry with nucleic acids are found, e.g., in Fantoni et al. (2021) Chem. Rev. 121(12):7122-7154.

Electric sources that may be employed in conjunction with the subject methods vary. In some cases, the electric source is a battery. Alternatively, devices may be configured for use with an alternating current wall socket electric source. However, devices may be configured for use with any suitable power source, including direct current sources. In some cases, the electric source is configured to intermittently provide an electrical bias between the two or more site-specifically metalized nucleic acid regions when the device is in use. In other cases, the electric source is configured to continuously provide an electrical bias between the two or more site-specifically metalized nucleic acid regions when the device is in use. In some instances, the one or more electric sources provide an electrical bias between the two or more site-specifically metalized nucleic acid regions using a fixed voltage. Alternatively, the one or more electric sources may provide an electrical bias between the two or more site-specifically metalized nucleic acid regions using a variable voltage.

In some cases, the scaffold is used in conjunction with a nanopore. For example, in some cases, an analyte may be translocated through the nanopore, thereby translocating the analyte through the opening. In certain cases, the nucleic acid scaffold and the nanopore are arranged such that the analyte translocates through the opening prior to translocating through the nanopore. Alternatively, the nucleic acid scaffold and the nanopore may be arranged such that the analyte translocates through the nanopore prior to translocating through the opening.

A suitable nanopore device may include a chamber including an aqueous solution and a membrane that separates the chamber into two sections, the membrane including a nanopore formed therein. Electrical measurements may be made using single channel recording equipment such as that described, e.g., in Lieberman et al. (2010) J. Am. Chem. Soc. 132(50):17961-72; Stoddart et al. (2009) PNAS106(19):7702-7; U.S. Pat. No. 9,481,908; and U.S. Patent Application Publication No. US2014/0051068; the disclosures of which are incorporated herein by reference in their entireties for all purposes. Alternatively, electrical measurements may be made using a multi-channel system, for example as described in U.S. Patent Application Publication No. US2015346149, the disclosure of which is incorporated herein by reference in its entirety for all purposes.

In nanopore-based analysis (e.g., sequencing), the nanopore provides the sole passage through which an ionic solution on the cis side of the membrane contacts the ionic solution on the trans side. A constant voltage bias (trans side positive) produces an ionic current through the nanopore and drives polynucleotides in the cis chamber through the pore to the trans chamber. A processive enzyme (e.g., a helicase, polymerase, nuclease, or the like) may be bound to the polynucleotide such that its step-wise movement controls and ratchets the nucleotides through the small-diameter nanopore, nucleobase by nucleobase.

Suitable conditions for nanopore-facilitated analysis (e.g., protein pores, solid state pores, etc.) are known in the art. Typically, a voltage is applied across the membrane and pore. The voltage used may be from +2 V to −2 V, e.g., from −400 mV to +400 mV. The voltage used may be in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage may be in the range of from 100 mV to 240 mV, e.g., from 120 mV to 220 mV.

In some embodiments, the rate at which the analyte is exposed to the opening is controlled using a processive enzyme. Non-limiting examples of processive enzymes that may be employed include polymerases (e.g., a phi29 or other suitable polymerase) and helicases, e.g., a Hel308 helicase, a RecD helicase, a Tral helicase, a Tral subgroup helicase, an XPD helicase, or the like. The processive enzyme may bind, e.g., the analyte, followed by the resulting complex being drawn to the nanopore, e.g., by a potential difference applied across the nanopore. In other embodiments, the processive enzyme may be located at the nanopore (e.g., attached to or adjacent to the nanopore) such that the processive enzyme binds, e.g., the nucleic acid upon arrival at the nanopore.

The nanopore may be present in a solid-state film, a biological membrane, or the like. In some embodiments, the nanopore is a solid-state nanopore. In other embodiments, the nanopore is a biological nanopore. The biological nanopore may be, e.g., an alpha-hemolysin-based nanopore, a Mycobacterium smegmatis porin A (MspA)-based nanopore, or the like.

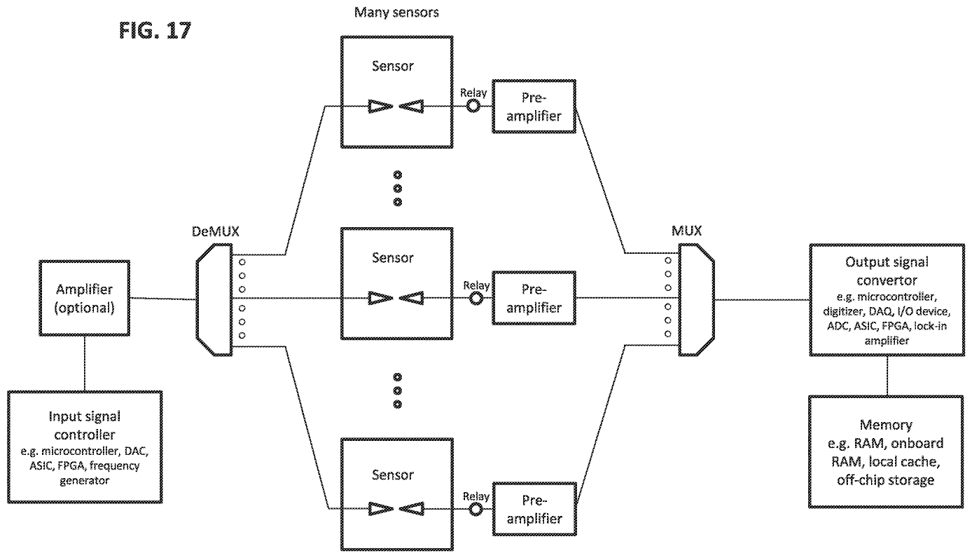

A device of the present disclosure is schematically illustrated in FIG. 17. In this example, the device comprises an input signal controller, an optional amplifier, and a demultiplexer (DeMUX) operably coupled to a plurality of sensors. Each sensor comprises a nucleic acid scaffold of the present disclosure. Each sensor may or may not comprise a nanopore aligned with the opening of the nucleic acid scaffold. When in use, conductance between two or more of the site-specifically metalized nucleic acid regions is conditioned upon a state of the structure having a fixed or variable resistance. As shown, the sensors are further operably coupled to a multiplexer (MUX), which is in turn operably coupled to an output signal converter which is operably coupled to a memory device.

A variety of processor-based systems may be employed to implement the embodiments of the present disclosure. Such systems may include system architecture wherein the components of the system are in electrical communication with each other using a bus. System architecture can include a processing unit (CPU or processor), as well as a cache, that are variously coupled to the system bus. The bus couples various system components including system memory, (e.g., read only memory (ROM) and random access memory (RAM), to the processor.

System architecture can include a cache of high-speed memory connected directly with, in close proximity to, or integrated as part of the processor. System architecture can copy data from the memory and/or the storage device to the cache for quick access by the processor. In this way, the cache can provide a performance boost that avoids processor delays while waiting for data. These and other modules can control or be configured to control the processor to perform various actions. Other system memory may be available for use as well. Memory can include multiple different types of memory with different performance characteristics. Processors can include any general purpose processor and a hardware module or software module, such as first, second and third modules stored in the storage device, configured to control the processor as well as a special-purpose processor where software instructions are incorporated into the actual processor design. The processor may essentially be a completely self-contained computing system, containing multiple cores or processors, a bus, memory controller, cache, etc. A multi-core processor may be symmetric or asymmetric.

Aspects of the invention also include non-transitory computer-readable media. The subject non-transitory computer-readable media include instructions stored thereon that cause a system to monitor a varying conductance along an analyte, or a varying conductance between the analyte and an electrode proximate to the analyte. As above, the varying conductance is indicative of sequential interactions between nucleobases of an analyte and one or more nucleobases of a scaffold. In select cases, the varying conductance comprises conductance fingerprints for the different nucleobases in the analyte, and the one or more non-transitory computer-readable media comprises instructions stored thereon that cause the system to determine the identity of one or more nucleotides of the analyte based on the varying conductance. In additional cases, the varying conductance comprises conductance fingerprints for the different nucleobases in the analyte, and the one or more non-transitory computer-readable media comprises instructions stored thereon that cause the system to determine a nucleotide sequence of the analyte based on the varying conductance.

To enable user interaction with the computing system architecture, an input device can represent any number of input mechanisms, such as a microphone for speech, a touch-sensitive screen for gesture or graphical input, keyboard, mouse, motion input, speech and so forth. An output device can also be one or more of a number of output mechanisms. In some instances, multimodal systems can enable a user to provide multiple types of input to communicate with the computing system architecture. A communications interface can generally govern and manage the user input and system output. There is no restriction on operating on any particular hardware arrangement and therefore the basic features here may easily be substituted for improved hardware or firmware arrangements as they are developed.

The storage device is typically a non-volatile memory and can be a hard disk or other types of computer-readable media which can store data that are accessible by a computer, such as magnetic cassettes, flash memory cards, solid state memory devices, digital versatile disks, cartridges, random access memories (RAMs), read only memory (ROM), and hybrids thereof.

The storage device can include software modules for controlling the processor. Other hardware or software modules are contemplated. The storage device can be connected to the system bus. In one aspect, a hardware module that performs a particular function can include the software component stored in a computer-readable medium in connection with the necessary hardware components, such as the processor, bus, output device, and so forth, to carry out various functions of the disclosed technology.

Embodiments within the scope of the present disclosure may also include tangible and/or non-transitory computer-readable storage media or devices for carrying or having computer-executable instructions or data structures stored thereon. Such tangible computer-readable storage devices can be any available device that can be accessed by a general purpose or special purpose computer, including the functional design of any special purpose processor as described above. By way of example, and not limitation, such tangible computer-readable devices can include RAM, ROM, EEPROM, CD-ROM or other optical disk storage, magnetic disk storage or other magnetic storage devices, or any other device which can be used to carry or store desired program code in the form of computer-executable instructions, data structures, or processor chip design. When information or instructions are provided via a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable storage devices.

Computer-executable instructions include, for example, instructions and data which cause a general purpose computer, special purpose computer, or special purpose processing device to perform a certain function or group of functions. Computer-executable instructions also include program modules that are executed by computers in stand-alone or network environments. Generally, program modules include routines, programs, components, data structures, objects, and the functions inherent in the design of special-purpose processors, etc. that perform tasks or implement abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

Other embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

In certain aspects, provided are one or more computer-readable media having stored thereon instructions for performing any of the steps of the methods of the present disclosure using any of the synthetic strands of the present disclosure. According to some embodiments, provided are the one or more computer-readable media of any of the systems of the present disclosure. For example, provided are one or more computer-readable media comprising instructions stored thereon, which when executed by one or more processors, cause the one or more processors to use one or more position indicator readers to determine the sequence of a nucleic acid by reading position indicators of a synthetic strand of the present disclosure during or subsequent to hybridization of the synthetic strand to the nucleic acid.

### Methods

Also provided by the present disclosure are methods. In certain embodiments, provided are methods implemented using any of the devices of the present disclosure, such methods comprising providing an electrical bias between the two or more site-specifically metalized nucleic acid regions, and measuring a conductance between the two or more site-specifically metalized nucleic acid regions.

According to some embodiments, the methods comprise causing relative movement of an analyte (e.g., a nucleic acid, protein to be sequenced) and an opening of the scaffold. By “causing relative movement”, it is meant that at least one of the scaffold and analyte changes location relative to the other. In some versions of the subject methods, the scaffold moves while the analyte remains stationary. In other embodiments, the analyte moves while the scaffold remains stationary. In still other embodiments, both the analyte and scaffold move. Where methods include causing the movement of the analyte relative to the scaffold, the movement may be effected using any convenient approach. For example, methods may include pulling the analyte through the opening. By “pulling”, it is meant exerting a force on the analyte sufficient to cause that analyte to change location. The requisite force may exist in any convenient form. For example, in some cases, the analyte is attached to an elongate structure which can be used to pull (i.e., physically pull) said nucleic acid. The force may be driven using any convenient approach, including but not limited to chemical propulsion, magnetic propulsion, ultrasound-driven propulsion, light-driven propulsion, electrically-driven propulsion, and combinations thereof. Nanoscale methods of propulsion are described in, e.g., Wang et al. Chemical reviews, 115(16), 8704-8735; herein incorporated by reference in its entirety. The speed of the relative movement may vary. In some instances, the speed of the relative movement ranges from 1 to 100,000 nucleotides/second.

In certain versions, the elongate structure is a nanowire. Nanowires may have any convenient diameter, such as where the diameter ranges from 0.5 nm to 500 nm, such as 1 nm to 200 nm, and including 5 nm to 100 nm. The nanowire may comprise any convenient material. Exemplary nanowire materials include, but are not limited to, carbon, germanium, silicon, gold, copper, yttrium barium copper oxide (YBCO), indium phosphide, gallium nitride, nickel, platinum, combinations thereof, and the like.

In additional embodiments, the elongate structure is a nanotube. A nanotube is a tube-like structure generally comprised of carbon (e.g., fullerene, graphene). Various techniques for producing carbon nanotubes have been developed. As examples, methods of forming carbon nanotubes are described in U.S. Pat. Nos. 5,753,088 and 5,482,601, the disclosures of which are hereby incorporated herein by reference. Non-limiting techniques for nanotube production include laser vaporization techniques, electric arc techniques, and gas phase techniques.

In still further embodiments, the elongate structure is a biopolymer. In some instances, the biopolymer is a protein. In other instances, the biopolymer is a nucleic acid. In other words, the biopolymer elongate structure may be employed in the same manner as the above-described nanotube or nanowire to pull the analyte. The biopolymer elongate structure may have any convenient amino acid or nucleotide structure, as desired.

Where methods include pulling the analyte via an elongate structure, the elongate structure may be attached to the analyte via any convenient approach. In some cases, an analyte may be bound (e.g., covalently bound) to an end of the elongate structure. In select cases, an adapter may be associated with one end of the analyte (e.g., at the 5′ end or the 3′ end of a nucleic acid). Any convenient adapter may be employed. In select versions, the elongate structure may have a complementary nucleic acid sequence to the adapter associated with the analyte such that the two molecules may hybridize when placed in proximity to one another, thereby attaching the elongate structure to the analyte. In an instance where the elongate structure is a nucleic acid, select nucleobases at the end of the elongate structure (e.g., at the 5′ end or the 3′ end) may be complementary to the to the adapter associated with the analyte such that the two molecules may hybridize when placed in proximity to one another.

In other cases, the force causing movement of the analyte relative to the scaffold is an electromagnetic force. In such cases, methods include applying a voltage across the opening such that the analyte moves relative to the scaffold. In some cases, the rate at which the analyte is pulled can be adjusted by adjusting the applied voltage. Voltages for use in the subject methods may vary, and in some instances may range from 25 mV to 500 mV, such as 50 mV to 400 mV, such as 75 mV to 300 mV and including 100 mV to 200 mV. In additional cases, the force causing movement of the analyte relative to the scaffold is an magnetic force. In some such cases, the analyte includes a magnetic particle (e.g., magnetic bead) attached thereto. The type of magnetic particle employed may vary, and can include, for example, iron nanoparticles, nickel nanoparticles, cobalt nanoparticles, and the like. In embodiments, applying a magnetic field to the magnetic particle attached to the analyte is sufficient to provide a pulling force to the analyte.

According to some embodiments, the methods of the present disclosure are computer-implemented. By “computer-implemented” is meant at least one step of the method is implemented using one or more processors and one or more non-transitory computer-readable media. The computer-implemented methods of the present disclosure may further comprise one or more steps that are not computer-implemented, e.g., obtaining a sample from a subject, isolating nucleic acids for sequencing, performing a contacting and/or combining step according to the methods of the present disclosure, and/or the like.

A nucleic acid to be sequenced according to the methods of the present disclosure may be a deoxyribonucleic acid (DNA). DNAs of interest include, but are not limited to, genomic DNA or fragments thereof, complementary DNA (or “cDNA”, synthesized from any RNA or DNA of interest) or fragments thereof, recombinant DNA (e.g., plasmid DNA) or fragments thereof, and/or the like. The nucleic acid to be sequenced may be greater than about 2 bases, greater than about 10 bases, greater than about 100 bases, greater than about 500 bases, greater than 1000 bases, greater than 10,000 bases, greater than 100,000 bases, greater than about 1,000,000, up to about 10¹⁰ or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

A nucleic acid to be sequenced according to the methods of the present disclosure may be a ribonucleic acid (RNA). The RNA may be any type of RNA (or sub-type thereof) including, but not limited to, a messenger RNA (mRNA), a microRNA (miRNA), a small interfering RNA (siRNA), a transacting small interfering RNA (ta-siRNA), a natural small interfering RNA (nat-siRNA), a ribosomal RNA (rRNA), a transfer RNA (tRNA), a small nucleolar RNA (snoRNA), a small nuclear RNA (snRNA), a long non-coding RNA (lncRNA), a non-coding RNA (ncRNA), a transfer-messenger RNA (tmRNA), a precursor messenger RNA (pre-mRNA), a small Cajal body-specific RNA (scaRNA), a piwi-interacting RNA (piRNA), an endoribonuclease-prepared siRNA (esiRNA), a small temporal RNA (stRNA), a signal recognition RNA, a telomere RNA, a ribozyme, or any combination of RNA types thereof or subtypes thereof.

In certain embodiments, moieties of the nucleic acid to be sequenced comprise a “non-natural nucleoside” or “non-natural nucleotide”, which refer to a nucleoside or nucleotide that contains a modified nucleobase and/or other chemical modification, such as a modified sugar. In some cases, non-natural nucleotides/nucleosides possess a unique conductance fingerprint that may be recognized by the subject methods. According to some embodiments, nucleic acids to be analyzed comprise moieties that comprise non-natural nucleobases and/or non-natural nucleotides that modify the melting temperature (Tm). Non-limiting examples include modified pyrimidine such as methyl-dC or propynyl-dU; modified purine, e.g., G-clamp; 2-Amino-2′-deoxyadenosine-5′-Triphosphate (2-Amino-dATP), 5-Methyl-2′-deoxycytidine-5′-Triphosphate (5-Me-dCTP), 5-Propynyl-2′-deoxycytidine-5′-Triphosphate (5-Pr-dCTP), 5-Propynyl-2′-deoxyuridine-5′-Triphosphate (5-Pr-dUTP), a halogenated deoxy-uridine (XdU) such as 5-Chloro-2′-deoxyuridine-5′-Triphosphate (5-CI-dUTP), 5-Bromo-2′-deoxyuridine-5′-Triphosphate (5-Br-dUTP), or any combination thereof.

A nucleic acid to be sequenced according to the methods of the present disclosure may be a nucleic acid from one or more immune cells. Immune cells of interest include, but are not limited to, T cells, B cells, natural killer (NK) cells, macrophages, monocytes, neutrophils, dendritic cells, mast cells, basophils, and eosinophils. In certain embodiments, the nucleic acid to be sequenced is from a T cell. T cells of interest include naive T cells (T_{N}), cytotoxic T cells (T_{CTL}), memory T cells (T_{MEM}), T memory stem cells (T_{SCM}), central memory T cells (T_{CM}), effector memory T cells (T_{EM}), tissue resident memory T cells (T_{RM}), effector T cells (T_{EFF}), regulatory T cells (T_{REGs}), helper T cells (T_{H}, T_{H}1, T_{H}2, T_{H}17) CD4+ T cells, CD8+ T cells, virus-specific T cells, alpha beta T cells (T_{αβ}), and gamma delta T cells (T_{γδ}).

In certain embodiments, a nucleic acid to be sequenced according to the methods of the present disclosure is a nucleic that encodes an immune cell receptor (e.g., a T cell receptor (TCR), a B cell receptor (BCR)) or a portion thereof. For example, in certain embodiments, provided are methods that comprise sequencing a nucleic acid that encodes one or more CDRs of an alpha chain or a beta chain of a TCR. According to some embodiments, the methods comprise sequencing a CDR3-encoding portion of a nucleic acid that encodes all or a portion of an alpha chain or a beta chain of a TCR.

The nucleic acids to be sequenced according to the methods of the present disclosure may be present in any nucleic acid sample of interest. In certain embodiments, the nucleic acids are present in a nucleic acid sample isolated from a single cell, a plurality of cells (e.g., cultured cells), a tissue, an organ, or an organism (e.g., bacteria, yeast, or the like). According to some embodiments, the nucleic acid sample is isolated from a cell(s), tissue, organ, and/or the like of an animal. In some embodiments, the animal is a mammal, e.g., a mammal from the genus Homo (e.g., a human), a rodent (e.g., a mouse or rat), a dog, a cat, a horse, a cow, or any other mammal of interest. In certain embodiments, the nucleic acid sample is isolated/obtained from a source other than a mammal, such as bacteria, yeast, insects (e.g., drosophila), amphibians (e.g., frogs (e.g., Xenopus)), viruses, plants, or any other non-mammalian nucleic acid sample source.

Nucleic acids that may be sequenced according to the methods of the present disclosure include cell-free nucleic acids, e.g., cell-free DNA, cell-free RNA, or both. Such cell-free nucleic acids may be obtained from any suitable source. In certain embodiments, the cell-free nucleic acids are from a body fluid sample selected from the group consisting of: whole blood, blood plasma, blood serum, amniotic fluid, saliva, urine, pleural effusion, bronchial lavage, bronchial aspirates, breast milk, colostrum, tears, seminal fluid, peritoneal fluid, pleural effusion, and stool. In certain embodiments, the cell-free nucleic acids are cell-free fetal DNAs. According to some embodiments, the cell-free nucleic acids are circulating tumor DNAs. In certain embodiments, the cell-free nucleic acids comprise infectious agent DNAs. According to some embodiments, the cell-free nucleic acids comprise DNAs from a transplant.

The term “cell-free nucleic acid” as used herein can refer to nucleic acid isolated from a source having substantially no cells. Cell-free nucleic acid may be referred to as “extracellular” nucleic acid, “circulating cell-free” nucleic acid (e.g., CCF fragments, ccf DNA) and/or “cell-free circulating” nucleic acid. Cell-free nucleic acid can be present in and obtained from blood (e.g., from the blood of an animal, from the blood of a human subject). Cell-free nucleic acid often includes no detectable cells and may contain cellular elements or cellular remnants. Non-limiting examples of acellular sources for cell-free nucleic acid are described above. Obtaining cell-free nucleic acid may include obtaining a sample directly (e.g., collecting a sample, e.g., a test sample) or obtaining a sample from another who has collected a sample. According to some embodiments, a cell-free nucleic acid may be a product of cell apoptosis and cell breakdown, which provides basis for cell-free nucleic acid often having a series of lengths across a spectrum (e.g., a “ladder”). In some embodiments, sample nucleic acid from a test subject is circulating cell-free nucleic acid. In some embodiments, circulating cell free nucleic acid is from blood plasma or blood serum from a test subject.

Cell-free nucleic acid can include different nucleic acid species, and therefore is referred to herein as “heterogeneous” in certain embodiments. For example, a sample from a subject having cancer can include nucleic acid from cancer cells (e.g., tumor, neoplasia) and nucleic acid from non-cancer cells. In another example, a sample from a pregnant female can include maternal nucleic acid and fetal nucleic acid. In another example, a sample from a subject having an infection or infectious disease can include host nucleic acid and nucleic acid from the infectious agent (e.g., bacteria, fungus, protozoa). In another example, a sample from a subject having received a transplant can include host nucleic acid and nucleic acid from the donor organ or tissue. In some instances, cancer, fetal, infectious agent, or transplant nucleic acid sometimes is about 5% to about 50% of the overall nucleic acid (e.g., about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49% of the total nucleic acid is cancer, fetal, infectious agent, or transplant nucleic acid). In another example, heterogeneous cell-free nucleic acid may include nucleic acid from two or more subjects.

Nucleic acids that may be sequenced according to the methods of the present disclosure include tumor nucleic acids (e.g., present in a nucleic acid sample isolated from a tumor—e.g., a tumor biopsy sample). “Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, prostate cancer, vulval cancer, thyroid cancer, hepatic carcinoma, various types of head and neck cancer, and the like.

Approaches, reagents and kits for isolating, purifying and/or concentrating DNA and RNA from sources of interest are known in the art and commercially available. For example, kits for isolating DNA from a source of interest include the DNeasy®, RNeasy®, QIAamp®, QIAprep® and QIAquick® nucleic acid isolation/purification kits by Qiagen, Inc. (Germantown, Md); the DNAzoI®, ChargeSwitch®, Purelink®, GeneCatcher@nucleic acid isolation/purification kits by Life Technologies, Inc. (Carlsbad, CA); the NucleoMag®, NucleoSpin®, and NucleoBond® nucleic acid isolation/purification kits by Clontech Laboratories, Inc. (Mountain View, CA). In certain embodiments, the nucleic acid is isolated from a fixed biological sample, e.g., formalin-fixed, paraffin-embedded (FFPE) tissue. Genomic DNA from FFPE tissue may be isolated using commercially available kits—such as the AllPrep® DNA/RNA FFPE kit by Qiagen, Inc. (Germantown, Md), the RecoverAll® Total Nucleic Acid Isolation kit for FFPE by Life Technologies, Inc. (Carlsbad, CA), and the NucleoSpin@FFPE kits by Clontech Laboratories, Inc. (Mountain View, CA).

Nucleic acid sequences determined according to the methods of the present disclosure may be analyzed (e.g., assembled and/or the like) using available sequence analysis software.

In select embodiments, the methods of the present disclosure include analyzing (e.g., sequencing) a polypeptide. Amino acids may possess conductance fingerprints in a manner similar to nucleotides. As such, encompassed by the present disclosure are embodiments in which the molecule to be analyzed (e.g., sequenced) is a polypeptide. A polypeptide to be analyzed may be any polypeptide, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The term “amino acid” generally refers to any monomer unit that comprises a substituted or unsubstituted amino group, a substituted or unsubstituted carboxy group, and one or more side chains or groups, or analogs of any of these groups. Exemplary side chains include, e.g., thiol, seleno, sulfonyl, alkyl, aryl, acyl, keto, azido, hydroxyl, hydrazine, cyano, halo, hydrazide, alkenyl, alkynl, ether, borate, boronate, phospho, phosphono, phosphine, heterocyclic, enone, imine, aldehyde, ester, thioacid, hydroxylamine, or any combination of these groups. Other representative amino acids include, but are not limited to, amino acids comprising photoactivatable cross-linkers, metal binding amino acids, spin-labeled amino acids, fluorescent amino acids, metal-containing amino acids, amino acids with novel functional groups, amino acids that covalently or noncovalently interact with other molecules, photocaged and/or photoisomerizable amino acids, radioactive amino acids, amino acids comprising biotin or a biotin analog, glycosylated amino acids, other carbohydrate modified amino acids, amino acids comprising polyethylene glycol or polyether, heavy atom substituted amino acids, chemically cleavable and/or photocleavable amino acids, carbon-linked sugar-containing amino acids, redox-active amino acids, amino thioacid containing amino acids, and amino acids comprising one or more toxic moieties.

The term “amino acid” includes, but is not limited to, naturally-occurring α-amino acids and their stereoisomers. “Stereoisomers” of amino acids refer to mirror image isomers of the amino acids, such as L-amino acids or D-amino acids. For example, a stereoisomer of a naturally-occurring amino acid refers to the mirror image isomer of the naturally-occurring amino acid (i.e., the D-amino acid).

Naturally-occurring α-amino acids are those encoded by the genetic code as well as those amino acids that are later modified (e.g., hydroxyproline, γ-carboxyglutamate, and O-phosphoserine). Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (GIn), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

### Kits

Aspects of the present disclosure further include kits. In certain embodiments, the kits find use, e.g., in performing any of the methods of the present disclosure. According to some embodiments, a kit of the present disclosure includes one or more nucleic acid scaffolds of the present disclosure, e.g., having a structure with a fixed or variable resistance operably coupled to site-specifically metalized nucleic acid regions within the nucleic acid scaffold.

A kit of the present disclosure may include one or more reagents that find use in analyzing analytes of interest, e.g., nucleic acids, polypeptides, and/or the like. For example, a kit of the present disclosure may include a solution (e.g., a buffer solution) having a pH, salt concentration, one or more components (e.g., chelating agents), and/or the like useful for providing suitable conditions for preparing an analyte for analysis by using a nucleic acid scaffold of the present disclosure.

A kit of the present disclosure may further include instructions for performing any of the methods of the present disclosure, e.g., sequencing nucleic acids. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

For purposes of completeness, non-limiting aspects and embodiments of the present disclosure are further defined in the following numbered clauses.
1. A nucleic acid scaffold, comprising:
   a structure having a fixed or variable resistance operably coupled to site-specifically metalized nucleic acid regions within the nucleic acid scaffold,
   wherein the structure having a fixed or variable resistance comprises an opening formed by one or more non-metalized nucleic acid regions of the nucleic acid scaffold, and
   wherein when in use, conductance between two or more of the site-specifically metalized nucleic acid regions is conditioned upon a state of the structure having a fixed or variable resistance.
2. The nucleic acid scaffold of clause 1, wherein the one or more non-metalized nucleic acid regions forming the opening share one or more nucleic acids with the two or more site-specifically metalized nucleic acid regions.
3. The nucleic acid scaffold of clause 1 or clause 2, wherein the one or more non-metalized nucleic acid regions forming the opening comprise a nucleic acid hybridized to a nucleic acid of the two or more site-specifically metalized nucleic acid regions.
4. The nucleic acid scaffold of any one of clauses 1 to 3, wherein the two or more site-specifically metalized nucleic acid regions comprise:
   a first metalized field adjacent the opening at a first location of the opening; and
   a second metalized field adjacent the opening at a second location of the opening.
5. The nucleic acid scaffold of clause 4, wherein the first and second locations are at substantially opposite sides of the opening.
6. The nucleic acid scaffold of any one of clauses 1 to 5, wherein site-specifically metalized nucleic acid regions within the nucleic acid scaffold comprise metallized duplexes.
7. The nucleic acid scaffold of clause 6, wherein site-specific metallization of the duplexes is achieved by a provided density of the duplexes to be site-specifically metalized.
8. The nucleic acid scaffold of any one of clauses 1 to 7, wherein the opening has a largest dimension of from 1 to 100 nanometers (nm).
9. The nucleic acid scaffold of clause 8, wherein the opening has a largest dimension of from 2 to 10 nm.
10. The nucleic acid scaffold of any one of clauses 1 to 9, wherein the state of the structure having fixed or variable resistance is the presence or absence of an analyte within the opening.
11. The nucleic acid scaffold of clause 10, wherein the state of the structure having fixed or variable resistance is the presence or absence of an analyte within the opening.
12. The nucleic acid scaffold of clause 10 or 11, wherein the analyte is a polymer.
13. The nucleic acid scaffold of clause 12, wherein the opening is adapted for translocation of the polymer therethrough.
14. The nucleic acid scaffold of clause 13, wherein the polymer is a nucleic acid or protein.
15. The nucleic acid scaffold of clause 14, wherein the polymer is a nucleic acid, and wherein the opening is adapted to hydrogen bond to bases of the nucleic acid translocating therethrough.
16. The nucleic acid scaffold of any one of clauses 1 to 15, wherein the state of the structure having fixed or variable resistance is the presence or absence of a polymer translocating through the opening.
17. The nucleic acid scaffold of clause 16, wherein conductance between the two or more site-specifically metalized nucleic acid regions varies, or occurs only, in the presence of a nucleic acid translocating through the opening.
18. The nucleic acid scaffold of any one of clauses 1 to 17, wherein the scaffold is present in a vacuum.
19. The nucleic acid scaffold of any one of clauses 1 to 17, wherein the scaffold is immersed in a fluid.
20. The nucleic acid scaffold of clause 19, wherein the fluid is a gas.
21. The nucleic acid scaffold of clause 19, wherein the fluid is a liquid.
22. The nucleic acid scaffold of any one of clauses 19 to 21, wherein the temperature of the fluid is selected to affect the conductance.
23. A device comprising:
   the nucleic acid scaffold of any one of clauses 1 to 17;
   electrodes operably coupled to the two or more site-specifically metalized nucleic acid regions;
   one or more electric sources operably coupled to the electrodes;
   one or more processors; and
   one or more computer-readable media comprising instructions stored thereon, which when executed by the one or more processors, cause the device to:
      provide an electrical bias between the two or more site-specifically metalized nucleic acid regions; and
      measure a conductance between the two or more site-specifically metalized nucleic acid regions.
24. The device of clause 23, wherein the instructions, when executed by the one or more processors, cause the one or more electric sources to intermittently provide an electrical bias between the two or more site-specifically metalized nucleic acid regions when the device is in use.
25. The device of clause 23, wherein the instructions, when executed by the one or more processors, cause the one or more electric sources to continuously provide an electrical bias between the two or more site-specifically metalized nucleic acid regions when the device is in use.
26. The device of any one of clauses 23 to 25, wherein the one or more electric sources provide an electrical bias between the two or more site-specifically metalized nucleic acid regions using a fixed voltage.
27. The device of any one of clauses 23 to 25, wherein the one or more electric sources provide an electrical bias between the two or more site-specifically metalized nucleic acid regions using a variable voltage.
28. The device of any one of clauses 23 to 27, wherein the electrodes are operably coupled to the two or more site-specifically metalized nucleic acid regions via attachment nucleic acids.
29. The device of clause 28, wherein the attachment nucleic acids comprise docking nucleic acids immobilized on the surface of an electrode, and wherein nucleic acids of the nucleic acid scaffold hybridize to the docking nucleic acids.
30. The device of any one of clauses 23 to 29, wherein the opening has a largest dimension of from 1 to 100 nanometers (nm).
31. The nucleic acid scaffold of clause 30, wherein the opening has a largest dimension of from 2 to 10 nm.
32. The device of any one of clauses 23 to 31, wherein the device comprises a nanopore aligned with the opening.
33. The device of clause 32, wherein the nucleic acid scaffold is inserted in the nanopore.
34. The device of clause 32 or 33, wherein the device is adapted to translocate an analyte through the nanopore, thereby translocating the analyte through the opening.
35. The device of clause 34, wherein the analyte is a polymer.
36. The device of clause 35, wherein the polymer is a nucleic acid or a protein.
37. The device of any one of clauses 34 to 36, wherein the nucleic acid scaffold and the nanopore are arranged such that the analyte translocates through the opening prior to translocating through the nanopore.
38. The device of any one of clauses 34 to 36, wherein the nucleic acid scaffold and the nanopore are arranged such that the analyte translocates through the nanopore prior to translocating through the opening.
39. The device of any one of clauses 34 to 38, wherein conductance between the two or more site-specifically metalized nucleic acid regions varies, or occurs only, in the presence of the analyte translocating through the opening.
40. The device of any one of clauses 34 to 39, wherein conductance between the two or more site-specifically metalized nucleic acid regions increases in the presence of the analyte translocating through the opening.
41. The device of any one of clauses 34 to 40, wherein the instructions cause the device to measure conductance between the two or more site-specifically metalized nucleic acid regions during translocation of the analyte through the opening.
42. The device of clause 41, wherein the analyte is a polymer, and wherein the conductance is a varying conductance indicative of the monomers of the polymer sequentially translocating through the opening.
43. The device of clause 41, wherein the polymer is a nucleic acid.
44. The device of clause 43, wherein the conductance is a varying conductance indicative of the bases of the nucleic acid sequentially translocating through the opening.
45. The device of any one of clauses 41 to 44, wherein the instructions cause the device to determine a sequence of the polymer based on the measured varying conductance.
46. The device of any one of clauses 23 to 45, wherein the scaffold is present in a vacuum.
47. The device of any one of clauses 23 to 45, wherein the scaffold is immersed in a fluid.
48. The device of clause 47, wherein the fluid is a gas.
49. The device of clause 47, wherein the fluid is a liquid.
50. The device of any one of clauses 47 to 49, wherein the temperature of the fluid is selected to achieve a desired conductance between the two or more site-specifically metalized nucleic acid regions, optionally wherein the conductance is a baseline conductance when an analyte is not present within the opening.
51. The device of any one of clauses 47 to 50, wherein the fluid comprises ions.
52. The device of clause 53, wherein the device is adapted to provide ionic current flow through the opening.
53. The device of any one of clauses 47 to 52, wherein the fluid comprises an ionic concentration selected to achieve a desired conductance between the two or more site-specifically metalized nucleic acid regions, optionally wherein the conductance is a baseline conductance when an analyte is not present within the opening.
54. A method implemented using the device of any one of clauses 23 to 45, the method comprising:
   providing an electrical bias between the two or more site-specifically metalized nucleic acid regions; and
   measuring a conductance between the two or more site-specifically metalized nucleic acid regions.
55. The method according to clause 54, wherein the opening has a largest dimension of from 1 to 100 nanometers (nm).
56. The method according to clause 55, wherein the opening has a largest dimension of from 2 to 10 nm.
57. The method according to any one of clauses 54 to 56, wherein the device comprises a nanopore aligned with the opening.
58. The method according to clause 57, wherein the nucleic acid scaffold is inserted in the nanopore.
59. The method according to clause 57 or 58, comprising translocating an analyte through the nanopore, thereby translocating the analyte through the opening.
60. The method according to clause 59, wherein the analyte translocates through the opening prior to translocating through the nanopore.
61. The method according to clause 59, wherein the analyte translocates through the nanopore prior to translocating through the opening.
62. The method according to any one of clauses 57 to 61, wherein the analyte is a polymer.
63. The method according to clause 62, wherein the polymer is a nucleic acid or protein.
64. The method according to clause 63, wherein the polymer is a nucleic acid, and wherein the opening is adapted to hydrogen bond to bases of the nucleic acid translocating therethrough.
65. The method according to clause 64, wherein the hydrogen bonding reduces the translocation rate of the nucleic acid as compared to the translocation rate in the absence of the hydrogen bonding.
66. The method according to clause 64 or 65, wherein the hydrogen bonding positions the nucleic acid to facilitate analysis of the nucleic acid.
67. The method according to any one of clauses 59 to 66, wherein conductance between the two or more site-specifically metalized nucleic acid regions varies, or occurs only, in the presence of the analyte translocating through the opening.
68. The method according to any one of clauses 59 to 67, wherein the instructions cause the device to measure conductance between the two or more site-specifically metalized nucleic acid regions during translocation of the analyte through the opening.
69. The method according to clause 68, wherein the analyte is a polymer, and wherein the conductance is a varying conductance indicative of the monomers of the polymer sequentially translocating through the opening.
70. The method according to any one of clause 69, wherein the polymer is a nucleic acid, and wherein the conductance is a varying conductance indicative of the bases of the nucleic acid sequentially translocating through the opening.
71. The method according to clause 69 or 70, wherein the instructions cause the device to determine a sequence of the polymer based on the measured varying conductance.
72. The method according to any one of clauses 59 to 71, wherein the scaffold is present in a vacuum.
73. The method according to any one of clauses 59 to 71, wherein the scaffold is immersed in a fluid.
74. The method according to clause 73, wherein the fluid is a gas.
75. The method according to clause 73, wherein the fluid is a liquid.
76. The method according to any one of clauses 73 to 75, wherein the temperature of the fluid is selected to achieve a desired conductance between the two or more site-specifically metalized nucleic acid regions, optionally wherein the conductance is a baseline conductance when an analyte is not present within the opening.
77. The method according to any one of clauses 73 to 76, wherein the fluid comprises ions.
78. The method according to clause 77, wherein the device is adapted to provide ionic current flow through the opening.
79. The method according to any one of clauses 73 to 78, wherein the fluid comprises an ionic concentration selected to achieve a desired conductance between the two or more site-specifically metalized nucleic acid regions, optionally wherein the conductance is a baseline conductance when an analyte is not present within the opening.

The following examples are offered by way of illustration and not by way of limitation.

### EXPERIMENTAL

### Example 1—Nucleic Acid Scaffold Design

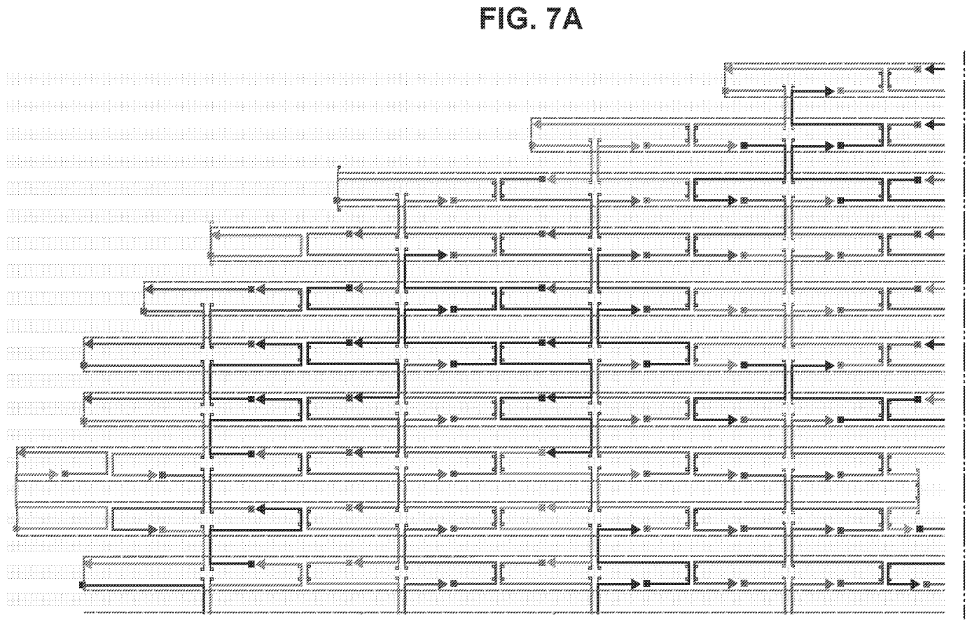

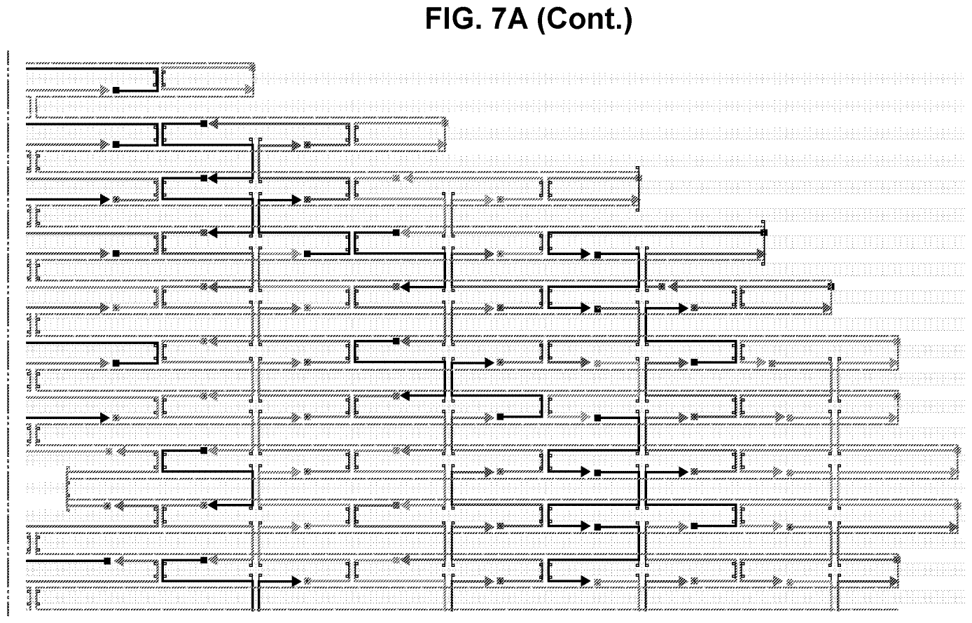

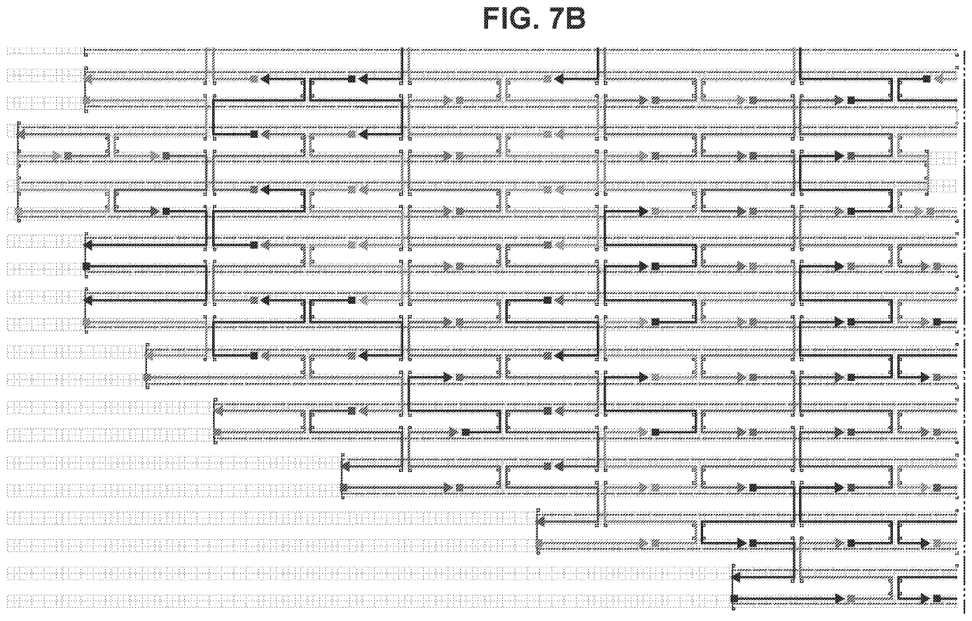

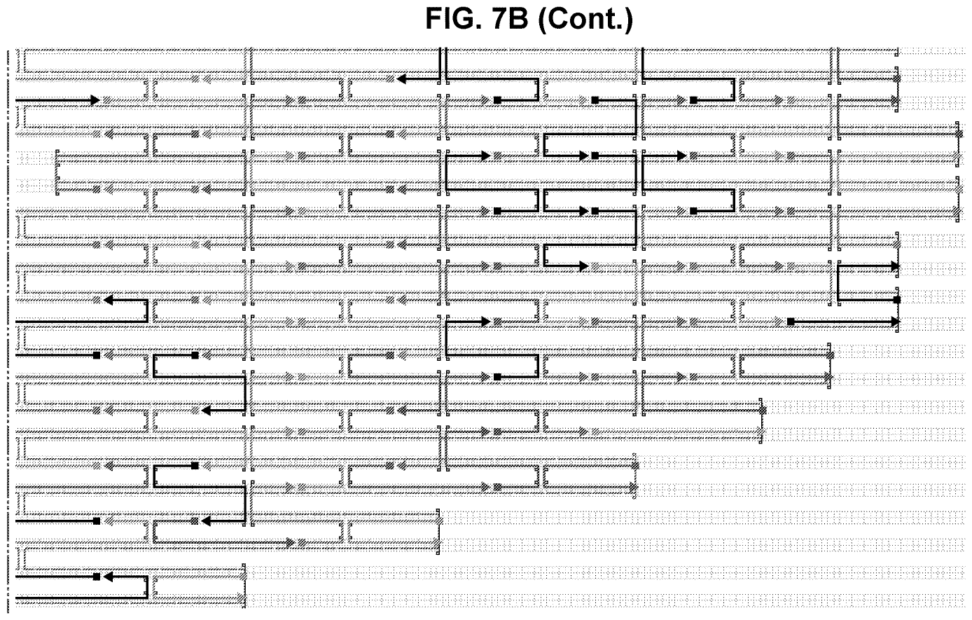

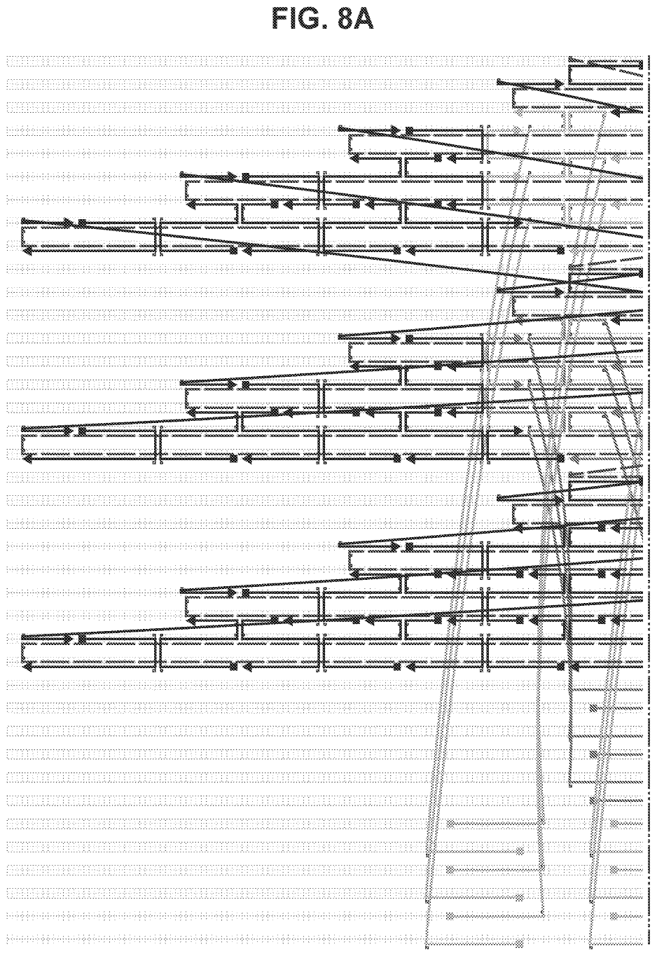

ENSnano and caDNAno depictions of a nucleic acid scaffold with a 4 nm pore are provided in FIGS. 6A-6B and FIGS. 7A-7B, respectively. Here, a planar DNA origami structure was designed on a “grid,” where each helix, depicted horizontally, is assigned a number and each nucleotide position is assigned an index number according to its vertical position in the design. The table of oligonucleotides below includes a start position (5′ end of the oligonucleotide) in which the helix number is listed first, and the index position is listed second and in brackets. Similarly, the end position (3′ end of the oligonucleotide) is listed with the helix number first and the index position second and in brackets. The oligonucleotides are shown on the 2D map and the 3D rendering (FIG. 6A-6B, 7A-7B), which are consistent between the two diagrams, and the scaffold is also shown. Each sphere in FIG. 6A-613 represents a nucleotide. The dark lines in FIG. 6A-613 represent locations where the scaffold or oligonucleotide must stretch to accommodate the design.

Details regarding the oligonucleotides employed in this design are provided in the table below.

| | | | | SEQ ID |
|---|---|---|---|---|
| Start | End | Length | Sequence | NO. |
| 31[256] | 33[255] | 32 | TAAATTGTTAGCTGATAAATTAATAAAGGGTG | 1 |
| 31[112] | 29[111] | 32 | CTGGCTGACGCGAAACAAAGTACAGGGTAAAA | 2 |
| 33[256] | 33[284] | 29 | AGAAAGGCCGGAGACAGTCAAATCACCAT | 3 |
| 11[128] | 13[127] | 32 | ATATTTAAATAAAGTACCGACAAATTACGAGC | 4 |
| 33[144] | 31[143] | 32 | GATTCATCTAACAAAGCTGCTCATTCTTGACA | 5 |
| 15[40] | 14[40] | 32 | TATCCGGTATTCTAAGTCAGATATAGAAGGCT | 6 |
| 28[47] | 26[48] | 32 | GCTTTTGCGCGGAGTGAGAATAGAGACGTTAG | 7 |
| 22[175] | 25[156] | 41 | GGGAGCTAAACAGATCAAAATCACCAGTGCCT | 8 |
| | | | TGAGTAGGA | |
| 26[191] | 28[192] | 32 | CTGTCGTGAAAACGACGGCCAGTGATGTGCTG | 9 |
| 24[223] | 26[224] | 32 | AGTCCACTTTCACCAGTGAGACGGAATTGCGT | 10 |
| 25[128] | 27[127] | 32 | CATTTTCAGTACAAACTACAACGCTCCAAAAG | 11 |
| 15[208] | 13[207] | 32 | ATTCATCAACAGTACCTTTTACATATTTCAAT | 12 |
| 37[93] | 37[111] | 19 | AGGTCTTTACCCTGACTAT | 13 |
| 31[144] | 29[143] | 32 | AGAACCGGACTCATCTTTGACCCCCAACCTAA | 14 |
| 37[112] | 35[111] | 32 | TATAGTCAAACGAGAATGACCATAGAGGGGGT | 15 |
| 29[112] | 27[111] | 32 | TACGTAATACAATGACAACAACCATCTCCAAA | 16 |
| 30[295] | 31[295] | 32 | ATTTTTGTTAAATCAGTTAAAATTCGCATTAA | 17 |
| 23[208] | 21[207] | 32 | GGTGCCGTACGAGCACGTATAACGCCAGAATC | 18 |
| 26[47] | 24[48] | 32 | TAAATGAAATAGGTGTATCACCGTAAGGATTA | 19 |
| 26[175] | 27[157] | 30 | ATTAATGAATCGGGTACCGTAACACAGCTT | 20 |
| 33[112] | 31[111] | 32 | AACTAACGGCCCTGACGAGAAACACGCATAGG | 21 |
| 17[240] | 15[239] | 32 | TATCTAAAACTCGTATTAAATCCTTTGGATTA | 22 |
| 27[158] | 26[176] | 34 | GCTTTTTTTCCCAGTCACGACGTTGTCCAGCTGC | 23 |
| 28[306] | 27[306] | 22 | GGTGCCGGAAATATCCGCTCAC | 24 |
| 15[256] | 17[255] | 32 | AAGGGTTATACAAACAATTCGACAATATCTTT | 25 |
| 18[223] | 20[224] | 32 | CGCCTGCACAGAGATAGAACCCTTTGCAACAG | 26 |
| 25[256] | 27[255] | 32 | CCTGGCCCTGGGGTGCCTAATGAGAGCTCGAA | 27 |
| 23[256] | 25[255] | 32 | GAGCTTGAGCCCGAGATAGGGTTGCTTCACCG | 28 |
| 26[63] | 28[64] | 32 | TCTTTCCAAAGGAACAACTAAAGGGAGGCTTG | 29 |
| 22[191] | 24[192] | 32 | TCGTTAGACCAAATCAAGTTTTTTTCCAACGT | 30 |
| 36[231] | 37[231] | 32 | CAGTTGATTCCCAATTGTTTCATTCCATATAA | 31 |
| 24[47] | 22[48] | 32 | GGATTAGCTCCTCATTAAAGCCAGCACCACCA | 32 |
| 37[208] | 35[207] | 32 | GTCTGGAACTGCGAACGAGTAGATTGAAAAGG | 33 |
| 20[223] | 22[224] | 32 | GAAAAACGTGTTTTTATAATCAGTTACTATGG | 34 |
| 15[80] | 13[79] | 32 | CCCGACTTAGGAATCATTACCGCGTCAACAAT | 35 |
| 13[176] | 12[158] | 34 | AAATCGCGTAAATCGTCGCTATTAATTAAGCATT | 36 |
| 19[19] | 20[19] | 42 | ATTACGCAGTATGTTAGCAAAACCATTACCATT | 37 |
| | | | AGCAAGGCC | |
| 17[29] | 18[19] | 32 | AATAACATAAACTGGCATGATTAAGACTCCTT | 38 |
| 20[95] | 22[96] | 32 | TCACCGTCAGCGTCAGACTGTAGCCAGAACCG | 39 |
| 16[223] | 18[224] | 32 | ACGTTATTCAGTTGAAAGGAATTGATTAACAC | 40 |
| 23[128] | 25[127] | 32 | ACTGGTAAAACAGTTAATGCCCCCACCACCCT | 41 |
| 10[231] | 11[231] | 32 | TTTATCAAAATCATAGAAGAGTCAATAGTGAA | 42 |
| 25[80] | 23[79] | 32 | CCTCAGAAGAAAGTATTAAGAGGCTCTGAATT | 43 |
| 19[128] | 21[127] | 32 | AATTCATAAGGTAAATATTGACGGTTTCGGTC | 44 |
| 35[144] | 33[143] | 32 | ATACTGCGATTCAACTAATGCAGAGGTAGAAA | 45 |
| 30[223] | 32[224] | 32 | TAGCCAGCGAAAAGCCCCAAAAACATTTTTGA | 46 |
| 16[295] | 17[295] | 32 | AATACATTTGAGGATTAGAGCCGTCAATAGAT | 47 |
| 27[272] | 25[271] | 32 | TAGCTGTTCATAAAGTGTAAAGCCTGAGAGAG | 48 |
| 23[112] | 21[111] | 32 | GATGATACTCAGAGCCGCCACCCTGCGTTTTC | 49 |
| 26[223] | 28[224] | 32 | TGCGCTCAGCAGGTCGACTCTAGATCTTCGCT | 50 |
| 31[128] | 33[127] | 32 | AAGAGTAATCAGTGAATAAGGCTTGAACAACA | 51 |
| 35[158] | 34[176] | 34 | TCATATAACCTGTTTAGCTATATTTTTAATACTT | 52 |
| 34[191] | 36[192] | 32 | TGACCCTGCATTTGGGGCGCGAGCTTAGTTTG | 53 |
| 34[175] | 35[157] | 30 | TTGCGGGAGAAGCAGGAATACCACGAATCG | 54 |
| 38[175] | 39[157] | 30 | AATTGCTGAATATAAGACTTCAAAAGGTCA | 55 |
| 24[95] | 26[96] | 32 | TGAAACATCCGCCACCCTCAGAACACAGCCCT | 56 |
| 39[158] | 38[176] | 34 | GGATTAGAGAGTACCTTTAATTGCTCTAGAGCTT | 57 |
| 28[175] | 29[157] | 30 | GGTAACGCCAGGGCGAGGTGAATTAGGCAA | 58 |
| 30[191] | 32[192] | 32 | AGTAACAAATGTCAATCATATGTAATTGCCTG | 59 |
| 29[158] | 28[176] | 34 | AAGAAACAAACGGCGGATTGACCGTATTAAGTTG | 60 |
| 16[157] | 17[175] | 30 | CTAACGAATAAGAGCAACTGAACCTCAAAT | 61 |
| 13[208] | 11[207] | 32 | TACCTGAGTCAATATATGTGAGTGAGATTAAG | 62 |
| 11[208] | 10[192] | 32 | ACGCTGAGGTCTGAGAGACTACCTTTTTAACC | 63 |
| 13[128] | 15[127] | 32 | ATGTAGAAAAGTACCGCACTCATCGTTGCTAT | 64 |
| 25[288] | 27[287] | 32 | ACGCTGGTACATACGAGCCGGAAGTCCTGTGT | 65 |
| 25[272] | 23[271] | 32 | TTGCAGCAATAAATCAAAAGAATACGGGGAAA | 66 |
| 23[240] | 21[239] | 32 | GGGAGCCCGCCGCTACAGGGCGCGGAGGCCAC | 67 |
| 31[272] | 29[271] | 32 | ATATTTTGCTCATTTTTTAACCAACTCAGGAA | 68 |
| 8[199] | 9[199] | 32 | ACGCGAGAAAACTTTTAATCGCAAGACAAAGA | 69 |
| 23[32] | 25[31] | 32 | CAAATAAAGGGGTTTTGCTCAGTAATAAGTAT | 70 |
| 38[199] | 39[199] | 32 | ATTTTTGCGGATGGCTCTTTTGATAAGAGGTC | 71 |
| 25[208] | 23[207] | 32 | TTTTTCTTATTAAAGAACGTGGACGGGGTCGA | 72 |
| 10[157] | 11[175] | 30 | CATAATTTTAGGCAGAGTTTTCCCTTAGAA | 73 |
| 17[80] | 15[79] | 32 | CTGAACAAAAATAAGAAACGATTTGCGAACCT | 74 |
| 16[191] | 18[192] | 32 | ATCATTTTCTCAATCAATATCTGGAGCCAGCA | 75 |
| 27[128] | 29[127] | 32 | GAGCCTTTACCGATAGTTGCGCCGGCCACTAC | 76 |
| 25[32] | 26[19] | 29 | AGCCCGGATTTTCTGTATGGGATTTTGCT | 77 |
| 31[29] | 30[48] | 35 | GCGCAGACGGTCAATCATAAGGGAACCTGCTCCAT | 78 |
| 20[47] | 18[48] | 32 | CCAGTAGCCGTAGAAAATACATACGGAATACC | 79 |
| 20[157] | 21[175] | 30 | ATTCAAATCTTTTCATAGAGGCCGATTAAA | 80 |
| 17[208] | 15[207] | 32 | CAAATCAAAATTTTAAAAGTTTGATGATGGCA | 81 |
| 17[112] | 15[111] | 32 | TATCAGAGAAAATAAACAGCCATACCTTAAAT | 82 |
| 27[112] | 25[111] | 32 | AAAAAGGCCTGTAGCATTCCACAGCGCCACCC | 83 |
| 17[176] | 16[158] | 34 | ATCAAACCGCGGAACAAAGAAACCACCAGCAACG | 84 |
| 11[176] | 10[158] | 34 | TCCTTGAAAGGTTGGGTTATATAACTATAGGAAT | 85 |
| 15[240] | 13[239] | 32 | TACTTCTGTTTCAGGTTTAACGTCAACAAACA | 86 |
| 27[80] | 25[79] | 32 | ATAATAATGCGTAACGATCTAAAGACCGCCAC | 87 |
| 21[112] | 19[111] | 32 | ATCGGCATAAATTATTCATTAAAGTTTATTTT | 88 |
| 13[240] | 12[224] | 32 | TCAAGAAAAATTACCTTTTTTAATGGAAACAG | 89 |
| 9[176] | 8[158] | 34 | GCAAATCCTCAAATATATTTTAGTTAATTTCATC | 90 |
| 21[19] | 23[31] | 45 | GGAAACGTCACCAATGAAACCGCCAGCATTGACAGGA | 91 |
| | | | TTCACAAA | |
| 12[263] | 13[263] | 32 | TTAACAATTTCATTTGACAAAATTAATTACAT | 92 |
| 24[316] | 23[316] | 42 | CTGTTTGATGGTGGTTCCGAAAGGGAAGAAAGCGAAA | 93 |
| | | | GGAGC | |
| 23[272] | 21[271] | 32 | GCCGGCGACGCGTAACCACCACACGCAAATTA | 94 |
| 29[256] | 31[255] | 32 | GTATCGGCTAGGAACGCCATCAAACAAATATT | 95 |
| 29[19] | 30[29] | 32 | GAAAGACAGCATCGGAACGAGGCCGGAACGAG | 96 |
| 21[240] | 19[239] | 32 | CGAGTAAATATTACCGCCAGCCATCTGACCTG | 97 |
| 21[176] | 20[158] | 34 | GGGATTTTCGTCTGAAATGGATTATTTACGCGAC | 98 |
| 29[208] | 27[207] | 32 | ATGGGCGCAGCTGGCGAAAGGGGGCCAAGCTT | 99 |
| 14[223] | 16[224] | 32 | ATACAGTAATATAATCCTGATTGTTTGCCCGA | 100 |
| 19[208] | 17[207] | 32 | CTGGCCAAACAGTGCCACGCTGAGTCAGTTGG | 101 |
| 35[240] | 33[239] | 32 | ACATCCAAAATTAAGCAATAAAGCTGTAGGTA | 102 |
| 12[223] | 14[224] | 32 | TACATAAACAAAAGAAGATGATGAAGATGAAT | 103 |
| 21[80] | 19[79] | 32 | TTGCCTTTACCGACTTGAGCCATTATAAAAGA | 104 |
| 20[63] | 22[64] | 32 | AGAGCCAGTAATCAGTAGCGACAGGAGCCGCC | 105 |
| 24[191] | 26[192] | 32 | CAAAGGGCTTTGCGTATTGGGCGCCGGGAAAC | 106 |
| 39[144] | 37[143] | 32 | ACTCCAACTATCGCGTTTTAATTCCATCAAAA | 107 |
| 34[263] | 35[263] | 32 | GGCAAAGAATTAGCAATAAATCATACAGGCAA | 108 |
| 32[284] | 31[271] | 29 | CAATATGATATTCAACCGTTCAAACGTTA | 109 |
| 22[95] | 24[96] | 32 | CCACCCTCCAGTAAGCGTCATACATATTATTC | 110 |
| 19[112] | 17[111] | 32 | GTCACAATAAGAAAAGTAAGCAGAAGCGCTAA | 111 |
| 30[63] | 32[64] | 32 | CCGCGACCGAACTGACCAACTTTGTTTAATTT | 112 |
| 18[95] | 20[96] | 32 | CAAAGTTAGACACCACGGAATAAGGTGAATTA | 113 |
| 26[95] | 28[96] | 32 | CATAGTTATTTTTCACGTTGAAAATCGCCCAC | 114 |
| 39[125] | 39[143] | 19 | CGAACCAGACCGGAAGCAA | 115 |
| 16[95] | 18[96] | 32 | CCCAATCCAGTCAGAGGGTAATTGTAGCCGAA | 116 |
| 33[81] | 31[79] | 31 | GTTGGGAATTGGGCTTGAGATGGAAAGAGGA | 117 |
| 35[208] | 33[207] | 32 | TGGCATCAAAATCGGTTGTACCAATCATATAT | 118 |
| 14[284] | 15[255] | 45 | TTTGCACGTAAAACAGAAATAAAGAAATTGCGTAGATA | 119 |
| | | | ATAATGG | |
| 21[288] | 23[287] | 32 | TTCTTTGATGTAGCGGTCACGCTGACGTGGCG | 120 |
| 18[306] | 19[284] | 32 | TAGCCCTAAAACATCGCCATTAAAAATGCTAT | 121 |
| 29[80] | 27[79] | 32 | TTCATGAGGATATATTCGGTCGCTAATTGCGA | 122 |
| 11[144] | 9[143] | 32 | ACATGTAATACTAGAAAAAGCCTGATAAGGCG | 123 |
| 32[191] | 34[192] | 32 | AGAGTCTGAAAATTTTTAGAACCCAAACATTA | 124 |
| 24[175] | 22[176] | 32 | GTCTATCACTACGTGAACCATCACATCAGAGC | 125 |
| 33[208] | 31[207] | 32 | TTTAAATGCAAAGGCTATCAGGTCCCCCGGTT | 126 |
| 15[128] | 17[127] | 32 | TTTGCACCCTAATTTGCCAGTTACAGATAACC | 127 |
| 15[144] | 13[143] | 32 | ATTTTATCGAACGGGTATTAAACCACCAATCA | 128 |
| 11[93] | 12[96] | 19 | CAACGCTCAACTCCAGACG | 129 |
| 21[208] | 19[207] | 32 | CTGAGAAGCTCATGGAAATACCTAGGGACATT | 130 |
| 16[267] | 15[284] | 25 | ACTTGAACCTACCATATCAAAATTA | 131 |
| 25[144] | 23[143] | 32 | AAGCCCAATAACAGTGCCCGTATATAAGTTTT | 132 |
| 13[144] | 11[143] | 32 | ATAATCGGCCAGTAATAAGAGAATCAACGCCA | 133 |
| 14[95] | 16[96] | 32 | TTCATCGTGCGGGAGGTTTTGAAGTTATTTAT | 134 |
| 23[80] | 21[79] | 32 | TACCGTTCAGAGCCACCACCCTCAAATCAAGT | 135 |
| 26[306] | 25[316] | 32 | AATTCCACACATTGCCCCAGCAGGCGAAAATC | 136 |
| 19[256] | 21[255] | 32 | TGGCACAGGTAATATCCAGAACAAAGAGTCTG | 137 |
| 32[223] | 34[224] | 32 | GAGATCTACAATGCCTGAGTAATGCTCAGAGC | 138 |
| 23[288] | 25[287] | 32 | AGAAAGGAATCGGCAAAATCCCTTAGCGGTCC | 139 |
| 12[95] | 14[96] | 32 | ACGACAATCCTGAACAAGAAAAATTTTTTATT | 140 |
| 12[157] | 13[175] | 30 | TTCGAGCTGTCTTTCCTATACCAAGTTACA | 141 |
| 36[175] | 37[157] | 30 | CGCAAATGGTCAAAATATTCATTGAGGAAG | 142 |
| 25[8] | 23[15] | 40 | CGTCGAGAGGGTTGATCCAGGCGGATAAGTGCACGA | 143 |
| | | | TTGG | |
| 37[158] | 36[176] | 34 | CCCGAAATGCTGTAGCTCAACATGTTATACATTT | 144 |
| 30[47] | 28[48] | 32 | GTTACTTAGGTAGCAACGGCTACATAAAGGCC | 145 |
| 23[16] | 22[8] | 24 | CCTTGATAGGTTGAGGCAGGTCAG | 146 |
| 21[272] | 19[271] | 32 | ACCGTTGTACTATCGGCCTTGCTGACAATATT | 147 |
| 21[256] | 23[255] | 32 | TCCATCACCCGCCGCGCTTAATGCCCGATTTA | 148 |
| 24[63] | 26[64] | 32 | CTCAAGAGACTCAGGAGGTTTAGTTTTTGTCG | 149 |
| 32[63] | 34[61] | 35 | CAACTTTAACTGGCTCATTATACCAGACGACGATA | 150 |
| 17[272] | 16[268] | 20 | AATAGATTTAGAAGTATTAG | 151 |
| 22[316] | 21[306] | 32 | GGGCGCTAGGGCGCTGGCAAGTTAGTAATAAC | 152 |
| 9[125] | 11[127] | 35 | CCGTGTGATAATTTAGTATCATATGCGGAATCGCC | 153 |
| 35[128] | 37[127] | 32 | GACTGGATTTTAAACAGTTCAGAAGAAGCAAA | 154 |
| 37[128] | 38[125] | 19 | GCGGATTGGAGCTTCAAAG | 155 |
| 22[63] | 24[64] | 32 | ACCAGAACAATGGAAAGCGCAGTCTGAGACTC | 156 |
| 16[63] | 18[64] | 32 | ACGTCAAAAGACGGGAGAATTAACGAAACGCA | 157 |
| 25[157] | 24[176] | 35 | ACCCATCCAACGCGCGGGGAGAGGCGGGAAAAACC | 158 |
| 27[19] | 28[19] | 42 | AAACAACTTTCAACAGTTTCAGGGATCGTCACCCTCAG | 159 |
| | | | CAGC | |
| 35[61] | 35[79] | 19 | AAAACCAAAATAGCGAGAG | 160 |
| 28[95] | 30[96] | 32 | GCATAACCGAAGTTTCCATTAAACACGGAGAT | 161 |
| 15[112] | 13[111] | 32 | CAAGATTAGAGAACAAGCAAGCCGAATATCCC | 162 |
| 35[80] | 33[80] | 33 | GCTTTTGCATAACCCTCGTTTACCAGTCAGGAC | 163 |
| 31[240] | 29[239] | 32 | TGTATAAGAATAATTCGCGTCTGGTGCCAGTT | 164 |
| 19[144] | 17[143] | 32 | CAGCGCCAATAGCAATAGCTATCTTTGAGTTA | 165 |
| 19[272] | 17[271] | 32 | TTTGAATGACCGAACGAACCACCATAACAACT | 166 |
| 17[256] | 19[255] | 32 | AGGAGCACGCAGAAGATAAAACAGAGAATACG | 167 |
| 28[223] | 30[224] | 32 | ATTACGCCATCGTAACCGTGCATCCCTTCCTG | 168 |
| 28[63] | 30[64] | 32 | CAGGGAGTGAGGCTTTGAGGACTAGTCGAAAT | 169 |
| 35[112] | 33[111] | 32 | AATAGTAAGAATTACGAGGCATAGATAAAACG | 170 |
| 31[158] | 30[176] | 34 | ATTACAACGGTAATCGTAAAACTAGCCCCGTCGG | 171 |
| 30[175] | 31[157] | 30 | ATTCTCCGTGGGATACACTAAAACATATTC | 172 |
| 14[157] | 15[175] | 30 | TTCCAACTGAATCTTACAAGGAGCGGAATT | 173 |
| 19[240] | 17[239] | 32 | AAAGCGTAAGGTGAGGCGGTCAGTAGGAAGGT | 174 |
| 20[191] | 22[192] | 32 | CGCTCAATAGACAGGAACGGTACGTGCTTTCC | 175 |
| 23[144] | 21[143] | 32 | AACGGGGTCGGAACCAGAGCCACCCTTATTAG | 176 |
| 25[240] | 23[239] | 32 | TGATTGCCAGTGTTGTTCCAGTTTACCCTAAA | 177 |
| 21[144] | 19[143] | 32 | CGTTTGCCCCGATTGAGGGAGGGATGGTTTAC | 178 |
| 8[157] | 9[175] | 30 | TTCTGAGAATAAACACCTGTAAATGCTGAT | 179 |
| 21[128] | 23[127] | 32 | ATAGCCCCACCGGAACCGCCTCCCAGGAGTGT | 180 |
| 36[191] | 37[207] | 32 | ACCATTAGTTAAATATGCAACTAAAGTACGGT | 181 |
| 13[61] | 14[64] | 19 | GCGCCTGTTTACCCAATAG | 182 |
| 19[285] | 21[287] | 35 | TAGTCTTTAATGAGTAGAAGAACTCAAAGCAATAC | 183 |
| 13[80] | 12[61] | 35 | AGATAAGTAAACAACATGTTCAGCTAATGCAGAAC | 184 |
| 15[176] | 14[158] | 34 | ATCATCATGGATTCGCCTGATTGCTTTGATATCA | 185 |
| 27[240] | 25[239] | 32 | GGGTACCGTGAGCTAACTCACATTGCAACAGC | 186 |
| 33[40] | 32[40] | 32 | TTATGCGATTTTAAGAATCATTGTGAATTACC | 187 |
| 18[191] | 20[192] | 32 | GCAAATGAACGACCAGTAATAAAACATTTTGA | 188 |
| 14[63] | 16[64] | 32 | CAAGCAAAAACGCGAGGCGTTTTATTTGTTTA | 189 |
| 9[144] | 8[125] | 35 | TTAAATAACCTAAATTTAATGGTTTGAAATACCGA | 190 |
| 29[128] | 31[127] | 32 | GAAGGCACCAGCGATTATACCAAGCCTTCATC | 191 |
| 20[306] | 19[306] | 22 | ATCACTTGCCTGCGCGAACTGA | 192 |
| 29[240] | 27[239] | 32 | TGAGGGGAGCGATCGGTGCGGGCCGGATCCCC | 193 |
| 33[240] | 31[239] | 32 | AAGATTCAGCCGGAGAGGGTAGCTAGGAAGAT | 194 |
| 19[80] | 17[79] | 32 | AACGCAAACCAGAAGGAAACCGAGTGAACACC | 195 |
| 19[176] | 18[158] | 34 | CCAGTCACAAAATCTAAAGCATCACCTTGGAAAC | 196 |
| 17[128] | 19[127] | 32 | CACAAGAATACCGAAGCCCTTTTTCAATAGAA | 197 |
| 25[112] | 23[111] | 32 | TCAGAGCCTGCCTATTTCGGAACCTGGCTTTT | 198 |
| 11[112] | 10[93] | 35 | TTAATTGATTATACAAATTCTTACCAGTATAAAGC | 199 |
| 37[144] | 35[143] | 32 | AGATTAAGAATCCCCCTCAAATGCAGCGTCCA | 200 |
| 27[208] | 25[207] | 32 | GCATGCCTCTGCCCGCTTTCCAGTCAGGGTGG | 201 |
| 29[144] | 27[143] | 32 | AACGAAAGTCTTAAACAGCTTGATAATTGTAT | 202 |
| 27[144] | 25[143] | 32 | CGGTTTATCTGAGTTTCGTCACCAGGGATAGC | 203 |
| 22[47] | 20[48] | 32 | GAGCCGCCATCGATAGCAGCACCGCAAAATCA | 204 |
| 34[95] | 36[93] | 35 | ACACTATCAAAAGAAGTTTTGCCAAATCAAAAATC | 205 |
| 34[223] | 35[239] | 32 | ATAAAGCTATTCTACTAATAGTAGTAGCATTA | 206 |
| 16[47] | 16[29] | 19 | TAGCAGCCTTTACAGAGAG | 207 |
| 18[47] | 16[48] | 32 | CAAAAGAAAACAGGGAAGCGCATTAATGAAAA | 208 |
| 33[158] | 32[176] | 34 | GATTTCTTTATTTCAACGCAAGGATAGAGCAAAC | 209 |
| 10[191] | 12[192] | 32 | TCCGGCTTAACATAGCGATAGCTTAATAACCT | 210 |
| 27[288] | 29[287] | 32 | GAAATTGTCCAGGCAAAGCGCCATTCCAGCCA | 211 |
| 31[80] | 29[79] | 32 | CAGATGAATCGCCTGATAAATTGTAAGACTTT | 212 |
| 33[128] | 35[127] | 32 | TTATTACATACATAACGCCAAAAGAATGTTTA | 213 |
| 27[256] | 29[255] | 32 | TTCGTAATGCAACTGTTGGGAAGGCGACGACA | 214 |
| 29[288] | 29[306] | 19 | GCTTTCCGGCACCGCTTCT | 215 |
| 29[272] | 27[271] | 32 | GATCGCACTCGCCATTCAGGCTGCCATGGTCA | 216 |
| 18[157] | 19[175] | 30 | AATGAAAAGACAAAAGGATTGGCAGATTCA | 217 |
| 32[175] | 33[157] | 30 | AAGAGAATCGATGCCAAATCAACGAGTTGA | 218 |
| 31[208] | 29[207] | 32 | GATAATCATTTCATCAACATTAAATGGTGTAG | 219 |
| 32[95] | 34[96] | 32 | AGTAGTAAAGAAAAATCTACGTTATAAGAGCA | 220 |
| 14[191] | 16[192] | 32 | ACAATAACATTCCTGATTATCAGAGTAACATT | 221 |
| 17[144] | 15[143] | 32 | AGCCCAATAGCGTCTTTCCAGAGCCAGCTACA | 222 |
| 28[191] | 30[192] | 32 | CAAGGCGAATGGGATAGGTCACGTTGTGAGCG | 223 |
| 12[191] | 14[192] | 32 | TGCTTCTGCAGAGGCGAATTATTCCGGGAGAA | 224 |
| 22[223] | 24[224] | 32 | TTGCTTTGAAAGCACTAAATCGGAGGAACAAG | 225 |
| 18[63] | 20[64] | 32 | ATAATAACATAAAGGTGGCAACATTGGGAATT | 226 |
| 30[95] | 32[96] | 32 | TTGTATCACGGTGTACAGACCAGGCCAGAACG | 227 |
| 13[112] | 11[111] | 32 | ATCCTAATAGGTAAAGTAATTCTGAGTAGGGC | 228 |

### Example 2—Selective Metallization

### DNA Origami Preparation

Oligonucleotide sequences for the construction of a planar triangle DNA origami with 20 base-pair, high-density overhanging oligonucleotides on two sides (areas A and B) are reported in PMID: 34962699 (Dai et al (2022) Angew Chem Int Ed Engl. 61(11)). Constructs were prepared using circular, single-stranded M13mp18 DNA (N4040S, New England Biolabs) and oligonucleotides synthesized and delivered in a pool (oPools, Integrated DNA Technologies). Oligos were rehydrated to 500 nM in DNA Suspension Buffer, pH 8.0 (T0221, Teknova).

For origami formation, the following were combined: 5 μL M13mp18 ssDNA (250 ng/μL), 10 μL pooled oligonucleotides (500 nM), 25 μL origami buffer (40 mM Tris, 2 mM EDTA-2Na, 12.5 mM Mg(OAc)₂, pH 8, (Dai, et al.)), 10 μL PCR-certified water (W3330, Teknova). Reaction mixtures were vortexed, briefly centrifuged, and subjected to a cooling protocol of 85-25° C. at 0.2° C. per minute in a thermal cycler.

At the termination of cooling, reactions were filtered through Amicon Ultra-0.5 100 kD MWCO filters (UFC510008, Millipore Sigma). Filters were prepared by the addition of 500 μL origami buffer and centrifugation at 14,000×g for 10 minutes. Samples were brought to 300 μL by the addition of 250 μL origami buffer. This diluted origami is loaded into the prepared filter and centrifuged at 3,000×g for 5 minutes. Filters were washed three times with 300 μL origami buffer and centrifuged at 3,000×g for five minutes per wash. Finally, filters were inverted into collection tubes and centrifuged at 1,000×g for two minutes.

### Quantification

DNA origami was quantified by nanophotometer (NP80-Touch, Implen) with DNA origami buffer as a blank. DNA origami molarity was calculated using this concentration (in ng/μL), the average molar mass of each base (330 g per mole of bases), the total number of DNA bases present in each individual triangular origami structure with overhanging oligonucleotides (15883 bases), and Avogadro's number (6.02×10²³ origami structures per mole).

### Selective Metal-Ion Seeding

DNA origami was diluted to 1 nM in origami buffer to a final volume of 60 μL. Rhodium chloride hydrate (520772, Millipore Sigma) was rehydrated to 500 mM in PCR-certified water, then diluted to 300 mM. 1.25 μL of rhodium chloride hydrate solution (300 mM) was added to diluted DNA origami. Vials were gently vortexed and briefly centrifuged. Metal-ion seeding was carried out for 24 hours in the dark without agitation.

### Metal Ion Reduction

Sodium borohydride was prepared by weighing a mass of 20-100 mg of sodium borohydride granules (452874, Millipore Sigma) into a 50 mL conical vial on an analytical balance pre-treated with an antistatic gun (Zerostat 3 Milty). The vial was transferred to an ice bucket and ice-cold deionized ultrapure water (BP2819100, Fisher Scientific) was added slowly while stirring to a final concentration of 500 mM. After complete dissolution, the sodium borohydride solution was serially diluted to 50 μM in deionized ultrapure water, and 1.25 μL of this solution was added to each metallization reaction. Directly after addition of sodium borohydride solution, reactions were mixed by repeated pipetting with a P200. Reduction was carried out for 15 minutes in the dark.

### Confirmation by TEM Imaging

300-mesh copper TEM grids with a layer of ~5 nm carbon film over a layer of lacey carbon (Cu-300LD, Pacific Grid-Tech) were subjected to glow discharge at 10 mA for 30 seconds (PELCO easiGlow 91000). 5 μL of each metallization reaction was pipetted onto grids and incubated for 2 minutes. The solution was then wicked by contact with filter paper (1002055, Cytiva). Grids were washed by the addition of 5 μL PCR-certified water, which is immediately wicked with filter paper. Plated grids were dried at least 30 minutes at ambient temperature and imaged on a low-voltage electron microscope (LVEM5, Delong America). Resulting TEM images demonstrating selective metallization (dark contrast) of triangles are shown in FIG. 9A-9B.

### Example 3—Selective Binding

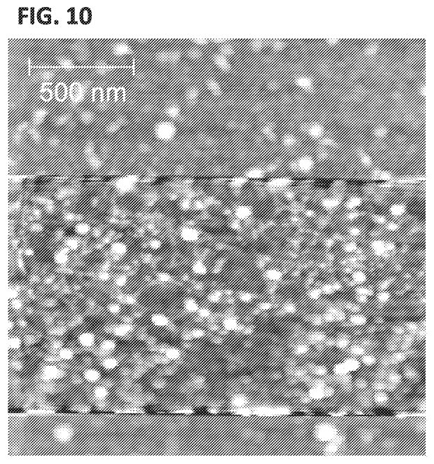

Chip-cleaning, pre-processing and DNA origami plating approaches were developed that resulted in triangle origami selectively binding to Au electrodes. A variety of DNA origami was tested for selective binding to Au electrodes. First, origami triangles functionalized with thiolated overhangs were tested. As shown in FIG. 10, selective binding of origami triangles to Au electrodes was achieved. Binding to either Au or SiN was not observed. When origami triangles functionalized with ss-overhangs were tested, origami was observed to bind to both Au and SiN. When origami triangles functionalized with poly(A) overhangs were tested, origami was observed to bind to both Au and SiN.

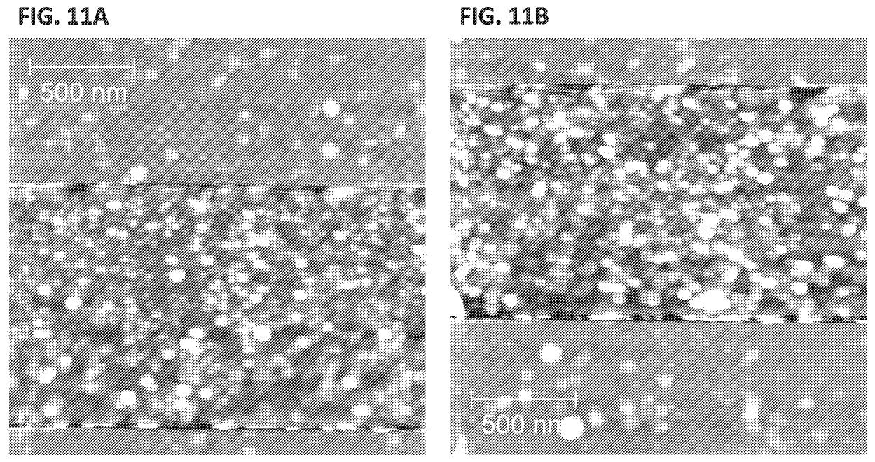

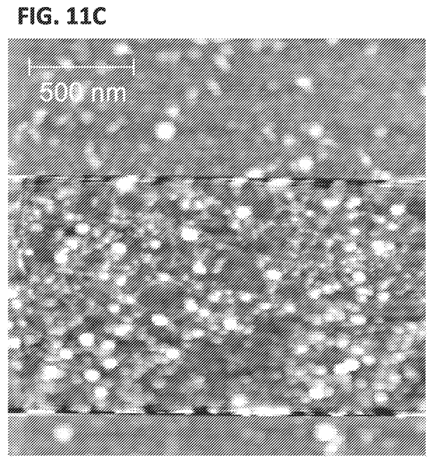

Selective binding of thiolated triangle origami to Au with sonication added to the chip cleaning approach was explored. Chip preparation included backside SiN removal, acetone/ipa resist strip, son. 10 min IPA (80 kHz), UV-ozone (15 min treat, 45 min soak), son. 20 min EtOH (80 kHz), EtOH wash. Plating involved: 1 hr TCEP (1 mM) activation, 2 hr incubation of 1.41 ng/μL origami in DSB-Mg (10 mM Tris-HCl, 0.1 mM EDTA, 12.5 mM MgCl₂) in solution cell on chip. The deposition area was washed with 8×15 DSB-Mg, then the chip was dipped 10 sec in each of 25%, 50%, 70%, 80%, 90% ethanol (in order). The chip was dried in air. All manipulations were at RT. Resulting images demonstrating selective binding of thiolated triangle origami to Au are shown in FIG. 11A-11C.

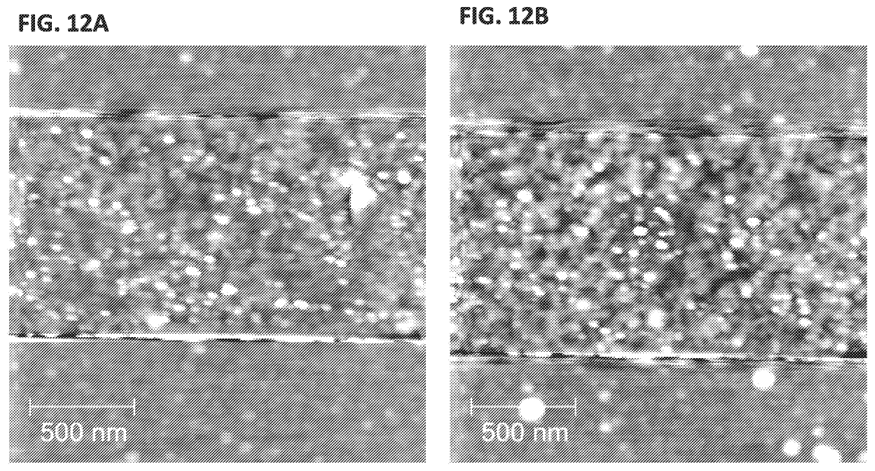

Selective binding of thiolated triangle origami to Au with gentler washing post-deposition was then explored. Chip preparation included: backside SiN removal, acetone/IPA resist strip, UV-ozone clean (15 min treat, 45 min soak), 20 min EtOH soak. Plating involved: 1 hr TCEP (1 mM) activation, 2 hr incubation of 1.41 ng/μL origami in DSB-Mg in solution cell on chip. The deposition area was washed with 2×5 μL DSB-Mg, 2×5 μL NFW. The chip was dried in air. All manipulations were at RT. Resulting images are shown in FIG. 12A-12C.

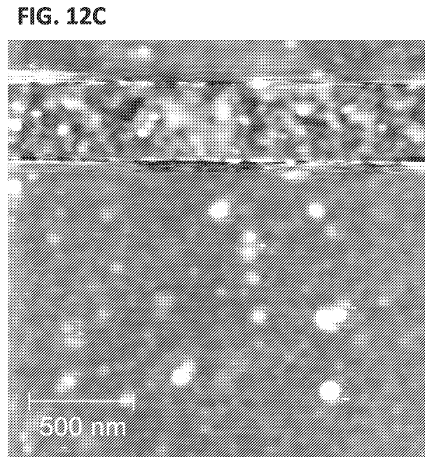

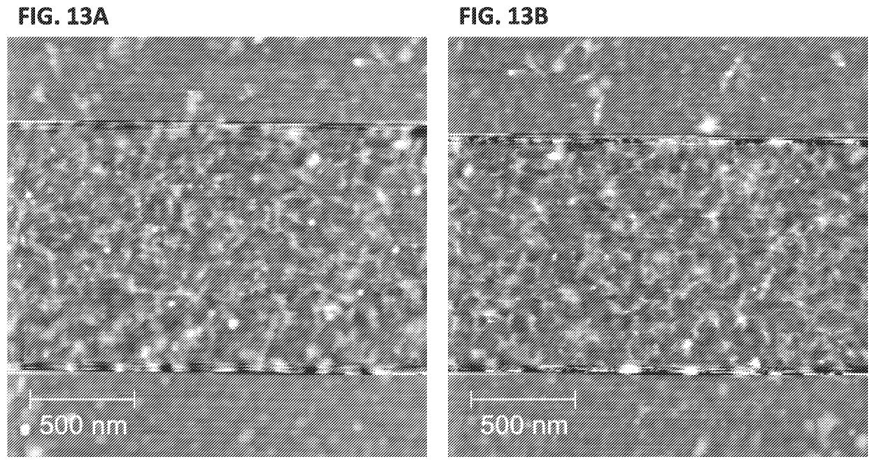

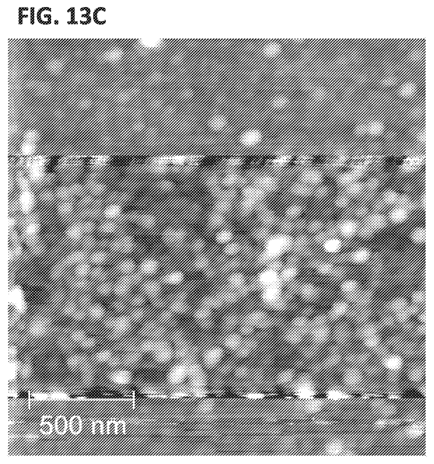

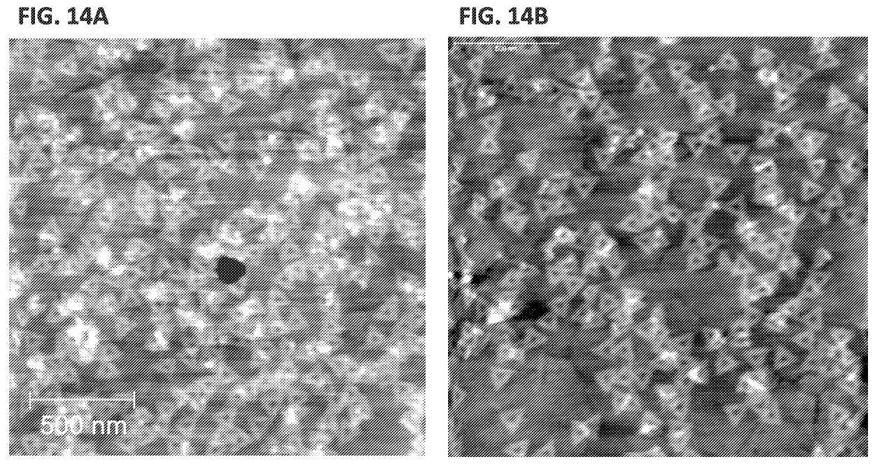

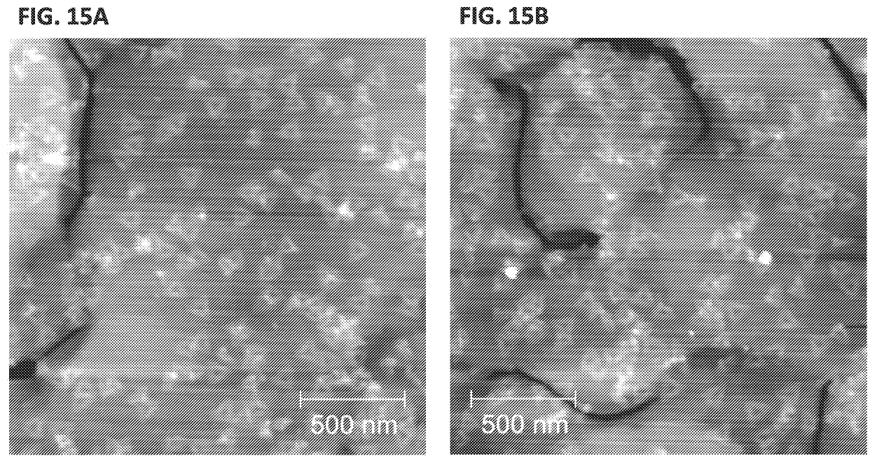

Selective binding of thiolated triangle origami to Au involving UV ozone cleaning with heat was also investigated. Chip preparation included: backside SiN removal, acetone/IPA resist strip, UV-ozone clean (15 min treat at 150° C., 45 min soak), 20 min EtOH soak. UV ozone cleaning was also performed with sonication, as follows: backside SiN removal, acetone/IPA resist strip, son 5 min IPA (37 kHz), son 10 min IPA (80 kHz), UV-ozone (15 min treat at 150° C., 45 min soak), son 5 min EtOH (37 kHz), son 20 min EtOH (80 kHz), EtOH wash. Plating involved 1 hr TCEP (1 mM) activation, 2 hr incubation of 1.41 ng/μL origami in DSB-Mg (10 mM Tris-HCl, 0.1 mM EDTA, 12.5 mM MgCl2) in solution cell on chip. Deposition area washed with 8×15 DSB-Mg, then chip dipped 10 sec in each of 25%, 50%, 70%, 80%, 90% ethanol (in order). Chip dried in air. All manipulations at RT. Images produced for the UV ozone cleaning and sonication condition are shown in FIG. 13A-13B, while FIG. 13C did not include sonication. Reference triangle origami images are shown in FIGS. 14A-14B and FIGS. 15A-15B.

### Example 4—DNA Origami Dimers

In this example, oligonucleotides (oligos) that are complementary to portions to M13mp18 single-stranded DNA (ssDNA) scaffold (New England Biolabs, N4040S) were modified from Dai, et al. (2022) Angew Chem Int Ed Engl. 2022 Mar. 7; 61(11):e202114190. doi: 10.1002/anie.202114190. Epub 2022 Jan. 27. PMID: 34962699 to allow the annealed scaffold and oligos to assume a triangle shape with single-stranded overhangs protruding up from one side of the triangle (AGACTAGACTAGACTAGACT; SEQ ID NO:229) and single-stranded cytosine overhangs protruding from the outside edge of another side of the triangle (CCCCCCCCCCCCCCC; SEQ ID NO:230). The oligos were synthesized in a pool (oPools, Integrated DNA Technologies) and rehydrated in 10 mM Tris-HCl, 0.1 mM EDTA, pH 8.0 (Teknova, T0258).

To fold the origami, five microliters (μL) of M13mp18 ssDNA (250 ng/μL) are combined with 10 μL pooled oligos (500 nM). The reaction volume was increased to 50 μL with 25 μL of origami buffer (10 mM 3-(N-morpholino)propanesulfonic acid (MOPS, Millipore-Sigma, M1442), 100 mM sodium nitrate (NaNO₃, Millipore-Sigma, S8170), 12.5 mM magnesium acetate (Sigma-Aldrich, 63052), pH 7.0) and 10 μL nuclease-free water (NFW, Teknova, W3332). The reaction was heated to 85° C. and cooled at a rate of 0.2° C. per minute to 25° C., then held at 10° C. The folded origami volume was increased to 250 μL with origami buffer and the solution was applied to an Amicon Ultra-0.5 100 kD molecular weight cut off (MWCO) filter (Amicon, UFC510008). The filter was washed three times with 300 μL origami buffer. Filtered DNA origami was quantified by nanophotometer at 260 nm (Implen, NP80).

Based on the expected silver-ion intercalation at the N3 sites of mismatched cytosine pairs, the number of potential cytosine-silver-cytosine (C—Ag+—C) mismatches was calculated from the molarity of DNA origami (calculated from the OD 260, 330 grams per mole of single-stranded bases, and 15313 single-stranded bases per origami) and the number of overhanging C bases (10 overhangs per triangle, 15 C's per overhang). Silver nitrate (Sigma-Aldrich, 85193) was added to folded DNA origami at a molar ratio of 1.5 silver ions (Ag+) to 1C-Ag+—C mismatch. Toomey et al. (2016) The Journal of Physical Chemistry 120(14):7804-7809 DOI: 10.1021/acs.jpcc.5b11968. The reaction was cooled from 45° C. to 25° C. at a rate of 1° C. per 6 minutes.

For plating and imaging, copper TEM grids (300-mesh) with a layer of lacey carbon and a layer of ~5 nm thin carbon (Grid-Tech, Cu-300LD) were glow discharged at 10 mA for 30 seconds. Five μL of silver-mediated origami dimers are plated on each grid for 2 minutes, wicked with Whatman filter paper (Cytiva Life Sciences, 1002-055), washed with 5 μL NFW, and wicked with Whatman filter paper. Plated origami is imaged on an LVEM5 TEM (DeLong America).

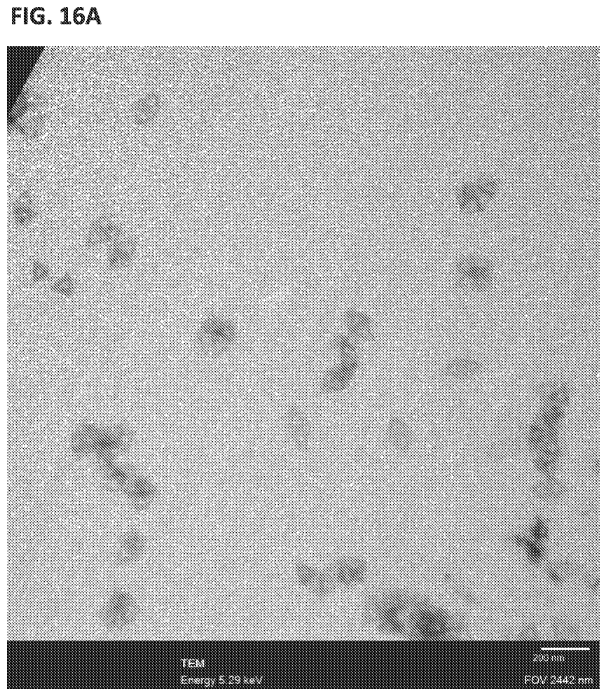

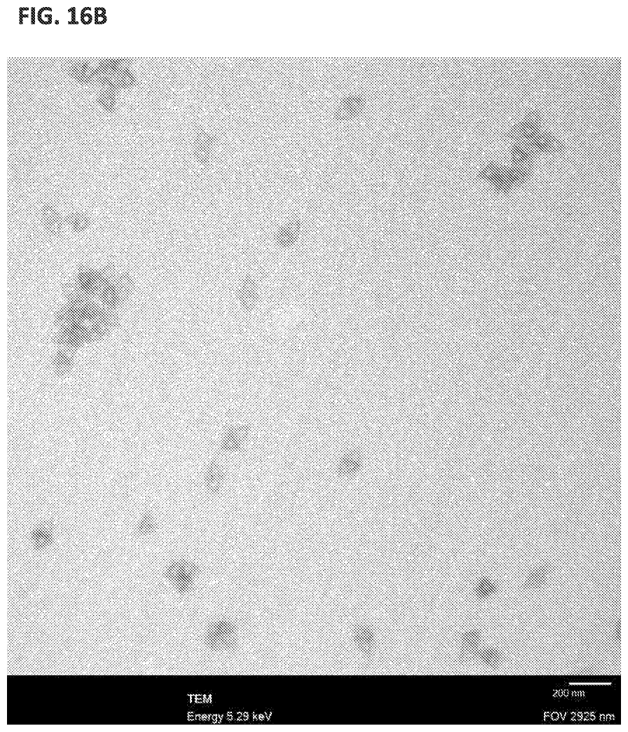

TEM images of dimers and clusters of DNA origami triangles joined by cytosine-cytosine mismatches mediated by silver ions in overhanging, single-stranded poly-cytosine oligonucleotides are shown in FIG. 16A-16B.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A nucleic acid scaffold, comprising:
a structure having a fixed or variable resistance operably coupled to site-specifically metalized nucleic acid regions within the nucleic acid scaffold,
wherein the structure having a fixed or variable resistance comprises an opening formed by one or more non-metalized nucleic acid regions of the nucleic acid scaffold, and
wherein when in use, conductance between two or more of the site-specifically metalized nucleic acid regions is conditioned upon a state of the structure having a fixed or variable resistance.

2. The nucleic acid scaffold of claim 1, wherein the one or more non-metalized nucleic acid regions forming the opening share one or more nucleic acids with the two or more site-specifically metalized nucleic acid regions.

3. The nucleic acid scaffold of claim 1 or claim 2, wherein the one or more non-metalized nucleic acid regions forming the opening comprise a nucleic acid hybridized to a nucleic acid of the two or more site-specifically metalized nucleic acid regions.

4. The nucleic acid scaffold of any one of claims 1 to 3, wherein the two or more site-specifically metalized nucleic acid regions comprise:
a first metalized field adjacent the opening at a first location of the opening; and
a second metalized field adjacent the opening at a second location of the opening.

5. The nucleic acid scaffold of claim 4, wherein the first and second locations are at substantially opposite sides of the opening.

6. The nucleic acid scaffold of any one of claims 1 to 5, wherein site-specifically metalized nucleic acid regions within the nucleic acid scaffold comprise metallized duplexes.

7. The nucleic acid scaffold of claim 6, wherein site-specific metallization of the duplexes is achieved by a provided density of the duplexes to be site-specifically metalized.

8. The nucleic acid scaffold of any one of claims 1 to 7, wherein the opening has a largest dimension of from 1 to 100 nanometers (nm).

9. The nucleic acid scaffold of claim 8, wherein the opening has a largest dimension of from 2 to 10 nm.

10. The nucleic acid scaffold of any one of claims 1 to 9, wherein the state of the structure having fixed or variable resistance is the presence or absence of an analyte within the opening.

11. The nucleic acid scaffold of claim 10, wherein the state of the structure having fixed or variable resistance is the presence or absence of an analyte within the opening.

12. The nucleic acid scaffold of claim 10 or 11, wherein the analyte is a polymer.

13. The nucleic acid scaffold of claim 12, wherein the opening is adapted for translocation of the polymer therethrough.

14. The nucleic acid scaffold of claim 13, wherein the polymer is a nucleic acid or protein.

15. The nucleic acid scaffold of claim 14, wherein the polymer is a nucleic acid, and wherein the opening is adapted to hydrogen bond to bases of the nucleic acid translocating therethrough.

16. The nucleic acid scaffold of any one of claims 1 to 15, wherein the state of the structure having fixed or variable resistance is the presence or absence of a polymer translocating through the opening.

17. The nucleic acid scaffold of claim 16, wherein conductance between the two or more site-specifically metalized nucleic acid regions varies, or occurs only, in the presence of a nucleic acid translocating through the opening.

18. The nucleic acid scaffold of any one of claims 1 to 17, wherein the scaffold is present in a vacuum.

19. The nucleic acid scaffold of any one of claims 1 to 17, wherein the scaffold is immersed in a fluid.

20. The nucleic acid scaffold of claim 19, wherein the fluid is a gas.

21. The nucleic acid scaffold of claim 19, wherein the fluid is a liquid.

22. A device comprising:
the nucleic acid scaffold of any one of claims 1 to 21;
electrodes operably coupled to the two or more site-specifically metalized nucleic acid regions;
one or more electric sources operably coupled to the electrodes;
one or more processors; and
one or more computer-readable media comprising instructions stored thereon, which when executed by the one or more processors, cause the device to:
provide an electrical bias between the two or more site-specifically metalized nucleic acid regions; and
measure a conductance between the two or more site-specifically metalized nucleic acid regions.

23. The device of claim 22, wherein the instructions, when executed by the one or more processors, cause the one or more electric sources to intermittently provide an electrical bias between the two or more site-specifically metalized nucleic acid regions when the device is in use.

24. The device of claim 22, wherein the instructions, when executed by the one or more processors, cause the one or more electric sources to continuously provide an electrical bias between the two or more site-specifically metalized nucleic acid regions when the device is in use.

25. The device of any one of claims 22 to 24, wherein the one or more electric sources provide an electrical bias between the two or more site-specifically metalized nucleic acid regions using a fixed voltage.

26. The device of any one of claims 22 to 24, wherein the one or more electric sources provide an electrical bias between the two or more site-specifically metalized nucleic acid regions using a variable voltage.

27. The device of any one of claims 22 to 26, wherein the electrodes are operably coupled to the two or more site-specifically metalized nucleic acid regions via attachment nucleic acids.

28. The device of claim 27, wherein the attachment nucleic acids comprise docking nucleic acids immobilized on the surface of an electrode, and wherein nucleic acids of the nucleic acid scaffold hybridize to the docking nucleic acids.

29. The device of any one of claims 22 to 28, wherein the opening has a largest dimension of from 1 to 100 nanometers (nm).

30. The nucleic acid scaffold of claim 29, wherein the opening has a largest dimension of from 2 to 10 nm.

31. The device of any one of claims 22 to 30, wherein the device comprises a nanopore aligned with the opening.

32. The device of claim 31, wherein the nucleic acid scaffold is inserted in the nanopore.

33. The device of claim 31 or 32, wherein the device is adapted to translocate an analyte through the nanopore, thereby translocating the analyte through the opening.

34. The device of claim 33, wherein the analyte is a polymer.

35. The device of claim 34, wherein the polymer is a nucleic acid or a protein.

36. The device of any one of claims 33 to 35, wherein the nucleic acid scaffold and the nanopore are arranged such that the analyte translocates through the opening prior to translocating through the nanopore.

37. The device of any one of claims 33 to 35, wherein the nucleic acid scaffold and the nanopore are arranged such that the analyte translocates through the nanopore prior to translocating through the opening.

38. The device of any one of claims 33 to 37, wherein conductance between the two or more site-specifically metalized nucleic acid regions varies, or occurs only, in the presence of the analyte translocating through the opening.

39. The device of any one of claims 33 to 38, wherein conductance between the two or more site-specifically metalized nucleic acid regions increases in the presence of the analyte translocating through the opening.

40. The device of any one of claims 33 to 39, wherein the instructions cause the device to measure conductance between the two or more site-specifically metalized nucleic acid regions during translocation of the analyte through the opening.

41. The device of claim 40, wherein the analyte is a polymer, and wherein the conductance is a varying conductance indicative of the monomers of the polymer sequentially translocating through the opening.

42. The device of claim 40, wherein the polymer is a nucleic acid.

43. The device of claim 42, wherein the conductance is a varying conductance indicative of the bases of the nucleic acid sequentially translocating through the opening.

44. The device of any one of claims 40 to 43, wherein the instructions cause the device to determine a sequence of the polymer based on the measured varying conductance.

45. The device of any one of claims 22 to 44, wherein the scaffold and electrodes are present in a vacuum.

46. The device of any one of claims 22 to 44, wherein the scaffold and electrodes are immersed in a fluid.

47. The device of claim 46, wherein the fluid is a gas.

48. The device of claim 46, wherein the fluid is a liquid.

49. The device of any one of claims 46 to 48, wherein the temperature of the fluid is selected to achieve a desired conductance between the two or more site-specifically metalized nucleic acid regions, optionally wherein the conductance is a baseline conductance when an analyte is not present within the opening.

50. The device of any one of claims 46 to 49, wherein the fluid comprises ions.

51. The device of claim 52, wherein the device is adapted to provide ionic current flow through the opening.

52. The device of any one of claims 46 to 51, wherein the fluid comprises an ionic concentration selected to achieve a desired and/or variable conductance between the two or more site-specifically metalized nucleic acid regions based on the ionic concentration/flow, optionally wherein the conductance is a baseline conductance when an analyte is not present within the opening.

53. The device of any one of claims 46 to 52, wherein the instructions, when executed by the one or more processors, cause the device to determine the temperature of the fluid based on the conductance.

54. A method implemented using the device of any one of claims 22 to 53, the method comprising:
providing an electrical bias between the two or more site-specifically metalized nucleic acid regions; and
measuring a conductance between the two or more site-specifically metalized nucleic acid regions.

55. The method according to claim 54, wherein the opening has a largest dimension of from 1 to 100 nanometers (nm).

56. The method according to claim 55, wherein the opening has a largest dimension of from 2 to 10 nm.

57. The method according to any one of claims 54 to 56, wherein the device comprises a nanopore aligned with the opening.

58. The method according to claim 57, wherein the nucleic acid scaffold is inserted in the nanopore.

59. The method according to claim 57 or 58, comprising translocating an analyte through the nanopore, thereby translocating the analyte through the opening.

60. The method according to claim 59, wherein the analyte translocates through the opening prior to translocating through the nanopore.

61. The method according to claim 59, wherein the analyte translocates through the nanopore prior to translocating through the opening.

62. The method according to any one of claims 57 to 61, wherein the analyte is a polymer.

63. The method according to claim 62, wherein the polymer is a nucleic acid or protein.

64. The method according to claim 63, wherein the polymer is a nucleic acid, and wherein the opening is adapted to hydrogen bond to bases of the nucleic acid translocating therethrough.

65. The method according to claim 64, wherein the hydrogen bonding reduces the translocation rate of the nucleic acid as compared to the translocation rate in the absence of the hydrogen bonding.

66. The method according to claim 64 or 65, wherein the hydrogen bonding positions the nucleic acid to facilitate analysis of the nucleic acid.

67. The method according to any one of claims 59 to 66, wherein conductance between the two or more site-specifically metalized nucleic acid regions varies, or occurs only, in the presence of the analyte translocating through the opening.

68. The method according to any one of claims 59 to 67, wherein the instructions cause the device to measure conductance between the two or more site-specifically metalized nucleic acid regions during translocation of the analyte through the opening.

69. The method according to claim 68, wherein the analyte is a polymer, and wherein the conductance is a varying conductance indicative of the monomers of the polymer sequentially translocating through the opening.

70. The method according to any one of claim 69, wherein the polymer is a nucleic acid, and wherein the conductance is a varying conductance indicative of the bases of the nucleic acid sequentially translocating through the opening.

71. The method according to claim 69 or 70, wherein the instructions cause the device to determine a sequence of the polymer based on the measured varying conductance.

72. The method according to any one of claims 59 to 71, wherein the scaffold and electrodes are present in a vacuum.

73. The method according to any one of claims 59 to 71, wherein the scaffold and electrodes are immersed in a fluid.

74. The method according to claim 73, wherein the fluid is a gas.

75. The method according to claim 73, wherein the fluid is a liquid.

76. The method according to any one of claims 73 to 75, wherein the temperature of the fluid is selected to achieve a desired conductance between the two or more site-specifically metalized nucleic acid regions, optionally wherein the conductance is a baseline conductance when an analyte is not present within the opening.

77. The method according to any one of claims 73 to 76, wherein the fluid comprises ions.

78. The method according to claim 77, wherein the device is adapted to provide ionic current flow through the opening.

79. The method according to any one of claims 73 to 78, wherein the fluid comprises an ionic concentration selected to achieve a desired and/or variable conductance between the two or more site-specifically metalized nucleic acid regions based on the ionic concentration/flow, optionally wherein the conductance is a baseline conductance when an analyte is not present within the opening.

80. The method according to any one of claims 73 to 79, comprising determining the temperature of the fluid based on the conductance.
